(19) United States
(12) Patent Application Publication
(10) Pub. No.: US 2009/0253744 A1
(43) Pub. Date: Oct. 8, 2009

(54) Acylated piperidine derivatives as melanocortin-4 receptor modulators

(76) Inventors: Raman K. Bakshi, Edison, NJ (US); James P. Dellureficio, Millington, NJ (US); Qingmei Hong, Scotch Plains, NJ (US); Tianying Jian, Westfield, NJ (US); Jian Liu, Edison, NJ (US); Ravi P. Nargund, East Brunswick, NJ (US); Zhixiong Ye, West Windsor, NJ (US)

Correspondence Address: MERCK AND CO., INC, P O BOX 2000, RAHWAY, NJ, 07065-0907, US

(21) Appl. No.: 12/311,006

(22) PCT Filed: Sep. 24, 2007
(86) PCT No.: PCT/US2007/020606

§ 371 Date: Mar. 16, 2009

(30) Foreign Application Priority Data
Sep. 27, 2006 (US) 60847494

(51) Int. Cl. A61K 31/4545; A61K 31/454; C07D 407/14; A61P 3/04

(52) U.S. Cl. 514/318; 514/326; 546/194; 546/209

(57) ABSTRACT

Certain novel N-acylated spiropiperidine derivatives are ligands of the human melanocortin receptor(s) and, in particular, are selective ligands of the human melanocortin-4 receptor (MC-4R). They are therefore useful for the treatment, control, or prevention of diseases and disorders responsive to the modulation of MC-4R, such as obesity, diabetes, nicotine addiction, alcoholism, sexual dysfunction, including erectile dysfunction and female sexual dysfunction.

### BACKGROUND OF THE INVENTION

Obesity is a major health concern in Western societies. It is estimated that about 97 million adults in the United States are overweight or obese. Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arteriosclerosis; heart disease; abnormal heart rhythms; and heart arrythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death.

Pro-opiomelanocortin (POMC) derived peptides are known to affect food intake. Several lines of evidence support the notion that the G-protein coupled receptors (GPCRs) of the melanocortin receptor (MC-R) family, several of which are expressed in the brain, are the targets of POMC derived peptides involved in the control of food intake and metabolism. A specific single MC-R that may be targeted for the control of obesity has not yet been identified, although evidence has been presented that MC-4R signalling is important in mediating feed behavior (S. Q. Giraudo et al., “Feeding effects of hypothalamic injection of melanocortin-4 receptor ligands,” Brain Research, 80: 302-306 (1998)). Evidence for the involvement of MC-R's in obesity includes: i) the agouti (AvY) mouse which ectopically expresses an antagonist of the MC-1R, MC-3R and -4R is obese, indicating that blocking the action of these three MC-R's can lead to hyperphagia and metabolic disorders; ii) MC-4R knockout mice (D. Huszar et al., Cell, 88: 131-141 (1997)) recapitulate the phenotype of the agouti mouse and these mice are obese; iii) the cyclic heptapeptide MT-II (a non-selective MC-1R, -3R, -4R, and -5R agonist) injected intracerebroventricularly (ICV) in rodents, reduces food intake in several animal feeding models (NPY, ob/ob, agouti, fasted) while ICV injected SHU-9119 (MC-3R and 4R antagonist; MC-1R and -5R agonist) reverses this effect and can induce hyperphagia; iv) chronic intraperitoneal treatment of Zucker fatty rats with an α-NDP-MSH derivative (HP228) has been reported to activate MC-1R, -3R, -4R, and -5R and to attenuate food intake and body weight gain over a 12-week period (I. Corcos et al., “HP228 is a potent agonist of melanocortin receptor-4 and significantly attenuates obesity and diabetes in Zucker fatty rats,” Society for Neuroscience Abstracts, 23: 673 (1997)).

Studies have shown that the melanocortin system contributes to the regulation of feeding behavior and bodyweight. Administration of melanocortin antagonists increases food intake and bodyweight, while administration of melanocortin agonists decreases food intake and bodyweight. Support for the role of the MC4R subtype in energy balance is demonstrated by evidence showing that the melanocortin-4 receptor deficiency in humans appears to be the most common monogenetic form of obesity with about 5-6% of obese patients showing this mutation. Furthermore, the severity of the phenotype appears to be greater in individuals that have mutations that result in complete loss of functioning. Based on these findings, the melanocortin system has been targeted for the development of small molecule agonists to treat obesity and small molecule antagonists to treat cachexia.

There is a need for a weight loss treatment with enhanced efficacy and fewer undesirable side effects. The instant invention addresses this problem by providing melanocortin receptor (MC-R) agonists, and in particular selective agonists of the melanocortin-4 receptor (MC-4R), useful in the treatment and prevention of obesity and obesity-related disorders, including diabetes.

Melanocortin receptor involvement in male and female sexual dysfunction has also been reported. Approximately 140 million men worldwide suffer from impotency or erectile dysfunction. Current treatment options for erectile dysfunction include phosphodiesterase V inhibitors, such as sildenafil citrate (Viagra®), vardenafil hydrochloride (Levitra®), and tadalafil (Clalis®). Sildenafil is effective in about 70% of patients, however it is contraindicated for patients with unstable heart conditions or cardiovascular disease, in particular patients taking nitrates, such as nitroglycerin, to treat angina. Vardenafil and Tadalafil are also contraindicated for patients taking nitrates and alpha blockers due to the risk of a sudden blood pressure drop resulting in fainting, heart attack or stroke. Other adverse effects associated with the clinical use of these PDE-5 inhibitors include headache, flushing, dyspepsia, dizziness, indigestion, and “abnormal vision, which is characterized by a bluish tinge to vision, but also an increased sensitivity to light or blurred vision. Sildenafil is also being evaluated for the treatment of female sexual dysfunction.

There is a need for a sexual dysfunction treatment with fewer undesirable side effects. The instant invention addresses this problem by providing melanocortin receptor (MC-R) agonists, and in particular selective agonists of the melanocortin-4 receptor (MC-4R), useful in the treatment and prevention of obesity and obesity-related disorders, including diabetes.

Synthetic melanocortin receptor agonists (melanotropic peptides) have been found to initiate erections in men with psychogenic erectile dysfunction. The centrally acting α-melanocyte-stimulating hormone analog, melanotan-II (MT-II), exhibited a 75% response rate when injected intramuscularly or subcutaneously into males with psychogenic erectile dysfunction [See H. Wessells et al., “Synthetic Melanotropic Peptide Initiates Erections in Men With Psychogenic Erectile Dysfunction: Double-Blind, Placebo Controlled Crossover Study,” J. Urol., 160: 389-393 (1998); Fifteenth American Peptide Symposium, Jun. 14-19, 1997 (Nashville Tenn.)]. MT-II (the cyclic heptapeptide Ac-Nle-c[Asp-His-DPhe-Arg-Trp-Lys]-NH₂) is a non-selective MC-1R, -3R, -4R, and —SR agonist (Dorr et al., Life Sciences, Vol. 58, 1777-1784, 1996). Adverse reactions observed with MT-II include nausea, flushing, loss of appetite, stretching, and yawning and may be the result of activation of MC-1R, MC-2R, MC-3R, and/or MC-5R. Additionally, MT-II must be administered parenterally, such as by subcutaneous, intravenous, or intramuscular route, since it is not absorbed into the systemic circulation when given by the oral route.

Compositions of melanotropic peptides and methods for the treatment of psychogenic erectile dysfunction are disclosed in U.S. Pat. No. 5,576,290. Methods of stimulating sexual response in females using melanotropic peptides have been disclosed in U.S. Pat. No. 6,051,555. Spiropiperidine, piperidine and piperazine derivatives have been disclosed in WO 99/64002; WO 00/74679; WO 01/58891; WO 01/70708; WO 01/70337; WO 01/91752; WO 02/015909; WO 02/059095; WO 02/059107; WO 02/059108; WO 02/059117; WO 02/067869, WO 02/068387; WO 02/068388; WO 02/070511; WO 02/079146; WO 02/085354; WO 03/061660, WO 03/000677; WO 03/007949; WO 03/009847; WO 03/009850; WO 03/068738; WO 03/092690; WO 03/093234; WO 03/094918; WO 04/024720; WO 04/048345; WO 04/058735; WO 04/078717; WO 04/112793; WO 04/224957; WO 04/089307; WO 04/078716; WO 04/078717; WO 04/087159; WO 05/042516; WO 05/040109; WO 05/077935, WO 05/009950; WO 05/040109; US2003096827; US2003092732; US2003232807, US2004224901, US2004/0097546, US2004/0092501, US2004/0204398, and US 20050176772 as agonists of the melanocortin receptor(s) and particularly as selective agonists of the MC-4R receptor and thereby useful for the treatment of diseases and disorders, such as obesity, diabetes, and sexual dysfunction, including erectile dysfunction and female sexual dysfunction.

Because of the unresolved deficiencies of the various pharmacological agents discussed above, there is a continuing need in the medical arts for improved methods and compositions to treat individuals suffering from psychogenic and/or organic sexual dysfunction. Such methods should have wider applicability, enhanced convenience and ease of compliance, short onset of action, reasonably long duration of action, and minimal side effects with few contraindications, as compared to agents now available. The present invention provide a acylated piperidine derivatives which are selective agonists of the melanocortin-4 (MC-4R) receptor and are useful to treat diseases associated with the melanocortin-4 receptor.

### SUMMARY OF THE INVENTION

The present invention relates to novel N-acylated spiropiperidines of structural formula I:

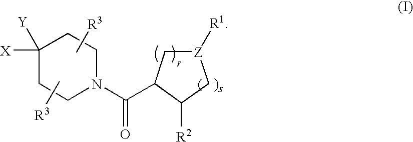

The compounds of structural formula I are effective as melanocortin receptor ligands and are particularly effective as selective ligands of the melanocortin-4 receptor. They are therefore useful for the treatment and/or prevention of disorders responsive to the modulation of the melanocortin-4 receptor, such as obesity, diabetes, obesity-related disorders, nicotine addiction, alcoholism, female sexual dysfunction, and male sexual dysfunction, in particular, male erectile dysfunction.

The present invention also relates to pharmaceutical compositions comprising the compounds of the present invention and a pharmaceutically acceptable carrier.

The present invention also relates to methods for the treatment or prevention of disorders, diseases, or conditions responsive to the modulation of the melanocortin-4 receptor in a mammal in need thereof by administering the compounds and pharmaceutical compositions of the present invention.

The present invention further relates to the use of the compounds of the present invention in the preparation of a medicament useful for the treatment or prevention of disorders, diseases, or conditions responsive to the modulation of the melanocortin-4 receptor in a mammal in need thereof by administering the compounds and pharmaceutical compositions of the present invention.

### DETAILED DESCRIPTION OF THE INVENTION

The present invention relates to N-acylated spiropiperidine derivatives useful as melanocortin receptor modulators, in particular, as selective melanocortin-4 receptor ligands. Compounds of the present invention are described by structural formula I:

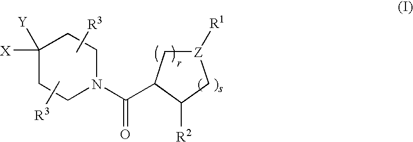

or a pharmaceutically acceptable salt thereof; wherein
X is selected from the group consisting of:

(1) —C₁₋₈ alkyl,

(2) —(CH₂)ₙC₃₋₈ cycloalkyl,

(3) —(CH₂)ₙ-phenyl,

(4) —(CH₂)ₙ-naphthyl,

(5) —(CH₂)ₙ-heteroaryl,

(6) —(CH₂)ₙheterocycloalkyl,

(7) —(CH₂)ₙC≡N,

(8) —(CH₂)ₙCON(R⁵)₂,

(9) —(CH₂)ₙCO₂R⁵,

(10) —(CH₂)ₙCOR⁵,

(11) —(CH₂)ₙNR⁵C(O)R⁵,

(12) —(CH₂)ₙNR⁵CO₂R⁵,

(13) —(CH₂)ₙNR⁵C(O)N(R⁵)₂,

(14) —(CH₂)ₙNR⁵SO₂R⁵,

(15) —(CH₂)ₙS(O)ₚR⁵,

(16) —(CH₂)ₙSO₂N(R⁵)₂,

(17) —(CH₂)ₙOR⁵,

(18) —(CH₂)ₙOC(O)R⁵,

(19) —(CH₂)ₙOC(O)OR⁵,

(20) —(CH₂)ₙOC(O)N(R⁵)₂,

(21) —(CH₂)ₙN(R⁵)₂, and

(22) —(CH₂)ₙNR⁵SO₂N(R⁵)₂,

wherein phenyl, naphthyl, and heteroaryl are unsubstituted or substituted with one to three groups independently selected from R⁴, and alkyl, cycloalkyl, and heterocycloalkyl are unsubstituted or substituted with one to three groups independently selected from R⁴ and oxo, and wherein any methylene (CH₂) in X is unsubstituted or substituted with one to two groups independently selected from halogen, hydroxy, and —C₁₋₆alkyl;
Y is selected from the group consisting of:

(1) hydrogen,

(2) —C₁₋₈ alkyl,

(3) —C₂₋₆ alkenyl,

(4) —(CH₂)ₙC₃₋₈ cycloalkyl,

(5) —(CH₂)ₙ-phenyl,

(6) —(CH₂)ₙ-naphthyl,

(7) —(CH₂)ₙ-heteroaryl, and

(8) —(CH₂)ₙ-heterocycloalkyl,

wherein alkenyl, phenyl, naphthyl, and heteroaryl are unsubstituted or substituted with one to three groups independently selected from R⁴, and alkyl, cycloalkyl, and heterocycloalkyl are optionally substituted with one to three groups independently selected from R⁴ and oxo, and wherein any methylene (CH₂) in Y is unsubstituted or substituted with one to two groups independently selected from halogen, hydroxy, and —C₁₋₆alkyl;
Z is selected from the group consisting of:

(1) —CH—, and

(2) —N—;

R¹ is selected from the group consisting of:

(1) —(CH₂)ₙC₂₋₇heterocycloalkyl,

(2) —(CH₂)ₙbridgedC₂₋₇heterocycloalkyl, and

(3) —N(R⁷)C₂₋₇heterocycloalkyl,

wherein heterocycloalkyl, and (CH₂)ₙ are unsubstituted or substituted with one to three groups independently selected from R⁹ and oxo, provided that Z and R¹ are not attached via a N—N bond;
R² is selected from the group consisting of:

(1) phenyl,

(2) naphthyl, and

(3) heteroaryl,

wherein phenyl, naphthyl, and heteroaryl are unsubstituted or substituted with one to four groups independently selected from R⁸;
each R³ is independently selected from the group consisting of:

(1) hydrogen,

(2) —OH,

(3) —C₁₋₈alkyl,

(4) —OC₁₋₈alkyl,

(5) halogen,

(6) —N(R⁵)₂,

(7) —SR⁵, and

(8) —CF₃,

wherein two C₁₋₈alkyl substituents along with the atoms to which they are attached can form a 4- to 8-membered cycloalkyl or heterocycloalkyl ring, and provided that when Z is —N—, Y is H or —OH, X is phenyl substituted with one to three R⁴ substituents and at least one R⁴ is —C₁₋₄alkyl, —(CH₂)₀₋₂C₃₋₅ cycloalkyl, halogen, —(CH₂)₀₋₃OR^{a}, CN, CO₂R^{b}, —(CH₂)₀₋₂NR^{b}SO₂R^{c}, CF₃, CH₂CF₃, OCF₃, or OCH₂CF₃, wherein R^{a}, R^{b} and R^{c} are —H, —CH₃, or —CH₂CH₃, then both R³ substituents are not methyl;
each R⁴ is independently selected from the group consisting of:

(1) —C₁₋₈ alkyl,

(2) —C₂₋₈ alkenyl,

(3) —(CH₂)ₙ-phenyl,

(4) —(CH₂)ₙ-naphthyl,

(5) —(CH₂)ₙ-heteroaryl,

(6) —(CH₂)ₙC₂₋₇ heterocycloalkyl,

(7) —(CH₂)ₙC₃₋₇ cycloalkyl,

(8) —(CH₂)ₙ-halogen,

(9) —(CH₂)ₙ—OR⁶,

(10) —(CH₂)ₙ—OSi(C₁₋₆alkyl)₃,

(11) —(CH₂)ₙC(O)R⁶,

(12) —(CH₂)ₙOC(O)R⁶,

(13) —(CH₂)ₙC(O)OR⁶,

(14) —(CH₂)ₙC≡N,

(15) —NO₂,

(16) —(CH₂)ₙN(R⁶)₂,

(17) —(CH₂)ₙC(O)N(R⁶)₂,

(18) —(CH₂)ₙNR⁶C(O)R⁶,

(19) —(CH₂)ₙNR⁶C(O)OR⁶,

(20) —(CH₂)ₙNR⁶C(O)-heteroaryl,

(21) —(CH₂)ₙNR⁶C(O)N(R⁶)₂,

(22) —(CH₂)ₙC(O)NR⁶N(R⁶)₂,

(23) —(CH₂)ₙC(O)NR⁶NR⁶C(O)R⁶,

(24) —(CH₂)ₙNR⁶S(O)ₚR⁶,

(25) —(CH₂)ₙS(O)ₚN(R⁶)₂,

(26) —(CH₂)ₙS(O)ₚR⁶,

(27) —O(CH₂)ₙC(O)N(R⁶)₂,

(28) —CF₃,

(29) —CH₂CF₃,

(30) —OCF₃, and

(31) —OCH₂CF₃,

wherein alkenyl, phenyl, naphthyl, heteroaryl are unsubstituted or substituted with one to three substituents independently selected from halogen, hydroxy, —C₁₋₆alkyl, trifluoromethyl, —C₁₋₆alkoxy, —CO₂C₁₋₆alkyl, and —CO₂H, and wherein any alkyl, cycloalkyl, heterocycloalkyl, and (CH₂) carbon atom in R⁴ is unsubstituted or substituted with one to two groups independently selected from halogen, hydroxy, oxo, —C₁₋₆alkyl, trifluoromethyl, —C₁₋₆alkoxy, —CO₂C₁₋₆alkyl, and —CO₂H, or two R⁴ substituents on the same carbon atom are taken together with the carbon atom to form a cyclopropyl group;
R⁵ is independently selected from the group consisting of

(1) hydrogen,

(2) —C₁₋₈alkyl,

(3) —C₂₋₈alkenyl,

(4) —C₂₋₈alkynyl,

(5) —C₁₋₈alkoxy,

(6) —(CH₂)ₙC₃₋₇cycloalkyl,

(7) —(CH₂)ₙC₂₋₇heterocycloalkyl,

(8) —(CH₂)ₙ-phenyl,

(9) —(CH₂)ₙ-naphthyl,

(10) —(CH₂)ₙ-heteroaryl, and

(11) —(CH₂)ₙC₃₋₇bicycloalkyl,

wherein alkenyl, alkynyl, phenyl, naphthyl, and heteroaryl are unsubstituted or substituted with one to three groups independently selected from R⁴, and alkyl, alkoxy, cycloalkyl, heterocycloalkyl, and bicycloalkyl are unsubstituted or substituted with one to three groups independently selected from R⁴ and oxo, and wherein any methylene (CH₂) in R⁵ is unsubstituted or substituted with one to two groups independently selected from halogen, hydroxy, and —C₁₋₆alkyl, or two R⁵ groups together with the atom to which they are attached form a 5- to 8-membered mono- or bicyclic ring system optionally containing an additional heteroatom selected from O, S, and —NC₁₋₄ alkyl;
each R⁶ is independently selected from the group consisting of:

(1) hydrogen,

(2) —C₁₋₆ alkyl,

(3) —(CH₂)ₙ-phenyl,

(4) —(CH₂)ₙ-heteroaryl,

(5) —(CH₂)ₙ-naphthyl,

(6) —(CH₂)ₙ-heterocycloalkyl,

(7) —(CH₂)ₙC₃₋₇cycloalkyl,

(8) —(CH₂)ₙC₃₋₇bicycloalkyl,

(9) —(CH₂)ₙCF₃, and

(10) —(CH₂)ₙCHF₂,

wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy, or two R⁶ groups together with the atom to which they are attached form a 4- to 8-membered mono- or bicyclic ring system optionally containing an additional heteroatom selected from O, S, and NC₁₋₄ alkyl;
each R⁷ is independently selected from the group consisting of:

(1) hydrogen, and

(2) —C₁₋₈alkyl,

wherein alkyl is unsubstituted or substituted with one to three groups independently selected from halogen, —C₁₋₆alkyl, hydroxy, and C₁₋₄ alkoxy;
each R⁸ is independently selected from the group consisting of:

(1) —C₁₋₆alkyl,

(2) —(CH₂)ₙphenyl,

(3) —(CH₂)ₙnaphthyl,

(4) —(CH₂)ₙheteroaryl,

(5) —(CH₂)ₙC₂₋₇heterocycloalkyl,

(6) —(CH₂)ₙC₃₋₇cycloalkyl,

(7) halogen,

(8) —OR⁶,

(9) —(CH₂)ₙN(R⁶)₂,

(10) —(CH₂)ₙC≡N,

(11) —(CH₂)ₙCO₂R⁶,

(12) —NO₂,

(13) —(CH₂)ₙNR⁶S(O)ₚR⁶,

(14) —(CH₂)ₙS(O)ₚN(R⁶)₂,

(15) —(CH₂)ₙS(O)ₚR⁶,

(16) —(CH₂)ₙNR⁶C(O)N(R⁶)₂,

(17) —(CH₂)ₙC(O)N(R⁶)₂,

(18) —(CH₂)ₙNR⁶C(O)R⁶,

(19) —(CH₂)ₙNR⁶CO₂R⁶,

(20) —(CH₂)ₙNR⁶C(O)-heteroaryl,

(21) —(CH₂)ₙC(O)NR⁶N(R⁶)₂,

(22) —(CH₂)ₙC(O)NR⁶NR⁶C(O)R⁶,

(23) —O(CH₂)ₙC(O)N(R⁶)₂,

(24) —CF₃,

(25) —CH₂CF₃,

(26) —OCF₃, and

(27) —OCH₂CF₃,

wherein phenyl, naphthyl, and heteroaryl are unsubstituted or substituted with one to three substituents independently selected from halogen, hydroxy, —C₁₋₆alkyl, trifluoromethyl, and C₁₋₄ alkoxy, and wherein alkyl, cycloalkyl, heterocycloalkyl, and (CH₂) are unsubstituted or substituted with one to three substituents independently selected from halogen, hydroxy, oxo, —C₁₋₆alkyl, trifluoromethyl, and C₁₋₄ alkoxy;
each R⁹ is independently selected from the group consisting of:

(1) —(CH₂)ₙ-halogen,

(2) —C₁₋₆alkyl,

(3) —(CH₂)ₙ—CO₂R⁶,

(4) —(CH₂)ₙ—OR⁶,

(5) —(CH₂)ₙ-phenyl,

(6) —(CH₂)ₙ-heteroaryl,

(7) —(CH₂)ₙ-naphthyl,

(8) —(CH₂)ₙ-heterocycloalkyl,

(9) —(CH₂)ₙC₃₋₇cycloalkyl,

(10) —(CH₂)ₙC₃₋₇bicycloalkyl,

(11) —(CH₂)ₙCF₃, and

(12) —(CH₂)ₙCHF₂,

wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy, or two R⁶ groups together with the atom to which they are attached form a 4- to 8-membered mono- or bicyclic ring system optionally containing an additional heteroatom selected from O, S, and NC₁₋₄ alkyl,
r is 1 or 2;
s is 0, 1, or 2;
n is 0, 1, 2, 3, or 4; and
p is 0, 1, or 2.

In a further embodiment of the compounds of the present invention, there are provided compounds of structural formula IIa or IIb of the indicated relative stereochemical configurations having the trans orientation of the R² and piperazinecarbonyl substituents:

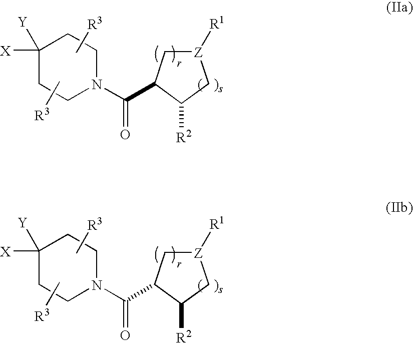

or a pharmaceutically acceptable salt thereof. In a class of this embodiment, R³ is hydrogen.

In a further embodiment of the compounds of the present invention, there are provided compounds of structural formula III:

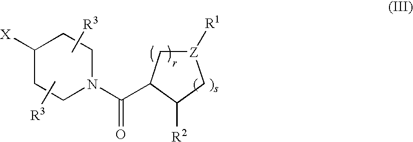

or a pharmaceutically acceptable salt thereof. In a class of this embodiment, R³ is hydrogen.

In yet a further embodiment of the compounds of the present invention, there are provided compounds of structural formula IVa or IVb of the indicated relative stereochemical configurations having the trans orientation of the phenyl and piperazinecarbonyl substituents:

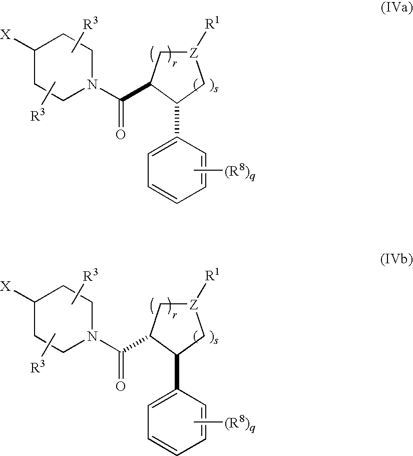

or a pharmaceutically acceptable salt thereof. In a class of this embodiment, R³ is hydrogen.

In yet a further embodiment of the compounds of the present invention, there are provided compounds of structural formula Va or Vb of the indicated relative stereochemical configurations having the trans orientation of the phenyl and piperazinecarbonyl substituents:

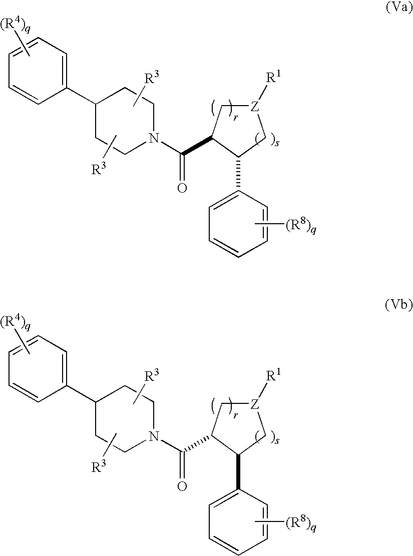

or a pharmaceutically acceptable salt thereof. In a class of this embodiment, R³ is hydrogen.

In one class of the embodiments of the present invention, X is selected from the group consisting of: —C₁₋₈ alkyl, —(CH₂)ₙC₃₋₈ cycloalkyl, —(CH₂)ₙ-phenyl, —(CH₂)ₙ-naphthyl, —(CH₂)ₙ-heteroaryl, —(CH₂)ₙheterocycloalkyl, —(CH₂)ₙC≡N, —(CH₂)ₙCON(R⁵)₂, —(CH₂)ₙCO₂R⁵, —(CH₂)ₙCOR⁵, —(CH₂)ₙNR⁵C(O)R⁵, —(CH₂)ₙNR⁵CO₂R⁵, —(CH₂)ₙNR⁵C(O)N(R⁵)₂, —(CH₂)ₙNR⁵SO₂R⁵, —(CH₂)ₙS(O)ₚR⁵, —(CH₂)ₙSO₂N(R⁵)₂, —(CH₂)ₙOR⁵, —(CH₂)ₙOC(O)R⁵, —(CH₂)ₙOC(O)OR⁵, —(CH₂)ₙOC(O)N(R⁵)₂, —(CH₂)ₙN(R⁵)₂, and —(CH₂)ₙNR⁵SO₂N(R⁵)₂, wherein phenyl, naphthyl, and heteroaryl are unsubstituted or substituted with one to three groups independently selected from R⁴, and alkyl, cycloalkyl, and heterocycloalkyl are unsubstituted or substituted with one to three groups independently selected from R⁴ and oxo, and wherein any methylene (CH₂) in X is unsubstituted or substituted with one to two groups independently selected from halogen, hydroxy, and —C₁₋₆alkyl.

In subclass of this class, X is selected from the group consisting of: —C₁₋₈ alkyl, —(CH₂)ₙphenyl, —(CH₂)ₙ-heteroaryl, —(CH₂)ₙNR⁵C(O)R⁵, and —(CH₂)ₙCON(R⁵)₂, wherein phenyl and heteroaryl are unsubstituted or substituted with one to three groups independently selected from R⁴, and alkyl is unsubstituted or substituted with one to three groups independently selected from R⁴ and oxo, and wherein any methylene (CH₂) in X is unsubstituted or substituted with one to two groups independently selected from halogen, hydroxy, and —C₁₋₆alkyl. In subclass of this class, X is selected from the group consisting of: —C₁₋₈ alkyl, —(CH₂)ₙphenyl, —(CH₂)ₙ-heteroaryl, and —(CH₂)ₙCON(R⁵)₂, wherein phenyl and heteroaryl are unsubstituted or substituted with one to three groups independently selected from R⁴, and alkyl is unsubstituted or substituted with one to three groups independently selected from R⁴ and oxo, and wherein any methylene (CH₂) in X is unsubstituted or substituted with one to two groups independently selected from halogen, hydroxy, and —C₁₋₆alkyl.

In another subclass of this class, X is selected from the group consisting of: —(CH₂)₂C(CH₃)₃, phenyl, -heteroaryl, and —C(O)NHC(CH₃)₃. In another subclass of this subclass, X is phenyl unsubstituted or substituted with one to three groups independently selected from R⁴. In another subclass of this subclass, X is —C(O)NHC(CH₃)₃. In a subclass of this subclass, X is heteroaryl unsubstituted or substituted with one to three groups independently selected from R⁴. In another subclass of this subclass, X is selected from phenyl and pyridyl, wherein phenyl and pyridyl are unsubstituted or substituted with one to three groups independently selected from R⁴.

In another class of the embodiments, X is selected from the group consisting of: —C₁₋₈alkyl, —(CH₂)₀₋₁phenyl, —(CH₂)₀₋₁pyridyl, —(CH₂)₀₋₃C(O)N(R⁵)₂, and —(CH₂)₀₋₃NR⁵C(O)R⁵; wherein phenyl and pyridyl are optionally substituted with one to three groups independently selected from R⁴; and CH₂ is unsubstituted or substituted with one to two groups independently selected from halogen, hydroxy, and —C₁₋₆alkyl. In another class of the embodiments, X is selected from the group consisting of: -phenyl, -pyridyl and —(CH₂)ₙCON(R⁵)₂, wherein phenyl and pyridyl are unsubstituted or substituted with one to three groups independently selected from R⁴, and alkyl is unsubstituted or substituted with one to three groups independently selected from R⁴ and oxo, and any methylene (CH₂) in X is unsubstituted or substituted with one to two groups independently selected from halogen, hydroxy, and —C₁₋₆alkyl. In a subclass of this class, X is phenyl optionally substituted with one to three groups independently selected from R⁴. In a subclass of this subclass, Y is hydrogen, and X is phenyl substituted with one to three groups independently selected from R⁴; or a pharmaceutically acceptable salt thereof. In another subclass of this class, X is selected from the group consisting of —(CH₂)₃NR⁵C(O)R⁵; wherein CH₂ is unsubstituted or substituted with one to two groups independently selected from halogen, hydroxy, and —C₁₋₆alkyl. In a subclass of this class, X is —CH(R⁴)—CH₂—C(CH₃)₂—NHC(O)CH₃, wherein R⁴ is neopentyl. In another subclass of this subclass, X is —CH(CH₂C(CH₃)₃)—CH₂C(CH₃)₂—NHC(O)CH₃. In another subclass of this class, X is —C(O)N(R⁵)₂; wherein CH₂ is unsubstituted or substituted with one to two groups independently selected from halogen, hydroxy, and —C₁₋₆alkyl. In a subclass of this subclass, Y is cyclohexane and X is —C(O)NH(C(CH₃)₃); or a pharmaceutically acceptable salt thereof. In another subclass of this class, X is pyridyl optionally substituted with one to three groups independently selected from R⁴. In a subclass of this subclass, Y is hydrogen, and X is pyridyl substituted with one to three groups independently selected from R⁴; or a pharmaceutically acceptable salt thereof.

In another class of the embodiments, Y is selected from the group consisting of: hydrogen, —C₁₋₈ alkyl, —C₂₋₆ alkenyl, —(CH₂)ₙC₃₋₈ cycloalkyl, —(CH₂)ₙ-phenyl, —(CH₂)ₙ-naphthyl, —(CH₂)ₙ-heteroaryl, and —(CH₂)ₙ-heterocycloalkyl; wherein alkenyl, phenyl, naphthyl, and heteroaryl are unsubstituted or substituted with one to three groups independently selected from R⁴, and alkyl, cycloalkyl, and heterocycloalkyl are optionally substituted with one to three groups independently selected from R⁴ and oxo; and wherein any methylene (CH₂) in Y is unsubstituted or substituted with one to two groups independently selected from halogen, hydroxy, and —C₁₋₆alkyl. In a subclass of this class, Y is selected from the group consisting of: hydrogen, —C₁₋₈ alkyl, and —(CH₂)ₙC₃₋₈ cycloalkyl, wherein alkyl, and cycloalkyl are optionally substituted with one to three groups independently selected from R⁴ and oxo; and wherein any methylene (CH₂) in Y is unsubstituted or substituted with one to two groups independently selected from halogen, hydroxy, and —C₁₋₆alkyl. In another subclass of this class, Y is selected from the group consisting of: hydrogen, —CH₂C(CH₃)₃, and cyclohexyl.

In another class of the embodiments, Y is hydrogen. In a subclass of this class, Y is hydrogen and X is phenyl. In another class of the embodiments, Y is —(CH₂)ₙC₃₋₈cycloalkyl, wherein cycloalkyl is optionally substituted with one to three groups independently selected from R⁴ and oxo; and wherein any methylene (CH₂) in Y is unsubstituted or substituted with one to two groups independently selected from halogen, hydroxy, and —C₁₋₆alkyl. In a subclass of this class, Y is cyclohexane. In another subclass of this class, Y is cyclohexane and X is —C(O)NH(C(CH₃)₃).

In another class of the embodiments, Z is selected from the group consisting of: —CH—, and —N—.

In another class of the embodiments, Z is —CH—. In a subclass of this class, Z is —CH— and R¹ is —NR⁷C₂₋₇heterocycloalkyl. In another subclass of this class, Z is —CH— and R¹ is —C₂₋₇heterocycloalkyl. In another class of the embodiments of the present invention, Z is —N—. In a subclass of this class, Z is —N— and R¹ is —(CH₂)ₙC₂₋₇heterocycloalkyl, provided that Z and R¹ are not attached via a N—N bond. In a subclass of this subclass, Z is —N— and R¹ is —C₂₋₇heterocycloalkyl, provided that Z and R¹ are not attached via a N—N bond.

In a class of the embodiment, R¹ is selected from the group consisting of: —(CH₂)ₙC₂₋₇heterocycloalkyl, —(CH₂)ₙbridgedC₂₋₇heterocycloalkyl, and —N(R⁷)C₂₋₇heterocycloalkyl, wherein heterocycloalkyl, and (CH₂)ₙ are unsubstituted or substituted with one to three groups independently selected from R⁹ and oxo, provided that Z and R¹ are not attached via a N—N bond.

In a subclass of this class, R¹ is selected from the group consisting of: azetidine, tetrahydropyran, tetrahydropyran amine, tetrahydropyran methyl amine, tetrahydrofuran methyl amine, tetrahydrofuran, pyrrolidine, piperidine, piperazine, morpholine, 2,5-diazabicyclo[2.2.1]heptane, 7-azabicyclo[2.2.1]heptane, 2-azabicyclo[2.2.1]heptane, 2-oxa-5-azabicyclo[2.2.1]heptane, -N(CH₃)tetrahydropyran, —N(H)tetrahydropyran, and —N(CH₃)tetrahydrofuran, wherein heterocycloalkyl, and (CH₂)ₙ are unsubstituted or substituted with one to three groups independently selected from R⁹ and oxo, provided that Z and R¹ are not attached via a N—N bond.

In another class of the embodiments, R¹ is selected from the group consisting of: —(CH₂)ₙC₂₋₇heterocycloalkyl, and —N(R⁷)C₂₋₇heterocycloalkyl, wherein heterocycloalkyl and (CH₂)ₙ are unsubstituted or substituted with one to three groups independently selected from R⁴ and oxo, provided that Z and R¹ are not attached via a N—N bond. In a subclass of this class, R¹ is —(CH₂)ₙC₂₋₇heterocycloalkyl, wherein heterocycloalkyl is unsubstituted or substituted with one to three groups independently selected from R⁴ and oxo, provided that Z and R¹ are not attached via a N—N bond. In a subclass of this subclass, R¹ is —C₂₋₇heterocycloalkyl and Z is —N— or —CH—, provided that Z and R¹ are not attached via a N—N bond. In another subclass of this class, R¹ is —N(R⁷)C₂₋₇heterocycloalkyl, wherein heterocycloalkyl is unsubstituted or substituted with one to three groups independently selected from R⁴ and oxo. In a subclass of this subclass, R¹ is —N(R⁷)C₂₋₇heterocycloalkyl and Z is —CH—.

In another class of the embodiments, R¹ is selected from the group consisting of: tetrahydrofuran, tetrahydropyran, pyrrolidine, piperidine, piperazine, morpholine, 2-oxa-5-azabicyclo[2.2.1]heptane, tetrahydropyran amine and tetrahydropyran methyl amine. In another class of the embodiments, R¹ is selected from the group consisting of: tetrahydropyran, pyrrolidine, 2-oxa-5-azabicyclo[2.2.1]heptane, and methyl tetrahydropyran amine, wherein R¹ is unsubstituted or substituted with one to three groups independently selected from R₄ and oxo. In another class of the embodiments, R¹ is tetrahydropyran. In another class of the embodiments, R¹ is selected from the group consisting of: pyrrolidine, piperidine, piperazine, morpholine, 2-oxa-5-azabicyclo[2.2.1]heptane, tetrahydropyran amine, and tetrahydropyran methyl amine.

In another class of the embodiments, R² is selected from the group consisting of: phenyl, naphthyl, and heteroaryl, wherein phenyl, naphthyl, and heteroaryl are unsubstituted or substituted with one to four groups independently selected from R⁸.

In another class of the embodiments, R² is phenyl unsubstituted or substituted with one to four groups independently selected from R⁸. In a subclass of this class, R² is phenyl substituted with one to three groups selected from C₁₋₄alkyl and halogen. In another subclass of this class, R² is phenyl substituted with one to three halogen groups. In a subclass of this class, R² is phenyl substituted with two fluorine groups. In another subclass of this class, R² is 2,4-difluorophenyl.

In another class of the embodiments, each R³ is independently selected from the group consisting of: hydrogen, —OH, —C₁₋₈alkyl, —OC₁₋₈alkyl, halogen, —N(R⁵)₂, —SR⁵, and —CF₃, wherein two C₁₋₈alkyl substituents along with the atoms to which they are attached can form a 4- to 8-membered cycloalkyl or heterocycloalkyl ring and provided that when Z is —N—, Y is H or —OH, X is phenyl substituted with one to three R⁴ substituents and at least one R⁴ is —C₁₋₄alkyl, —(CH₂)₀₋₂C₃₋₅ cycloalkyl, halogen, —(CH₂)₀₋₃OR^{a}, CN, CO₂R^{b}, —(CH₂)₀₋₂NR^{b}SO₂R^{c}, CF₃, CH₂CF₃, OCF₃, or OCH₂CF₃, wherein R^{a}, R^{b} and R^{c} are —H, —CH₃, or —CH₂CH₃, then both R³ substituents are not methyl. In a subclass of this class, each R³ is independently selected from the group consisting of: hydrogen, —OH, —C₁₋₈alkyl, —OC₁₋₈alkyl, halogen, —N(R⁵)₂, —SR⁵, and —CF₃, wherein two C₁₋₈alkyl substituents along with the atoms to which they are attached can form a 4- to 8-membered cycloalkyl or heterocycloalkyl ring, and provided that when Z is —N—, Y is H or —OH, X is phenyl substituted with one to three R⁴ substituents and at least one R⁴ is —C₁₋₄alkyl, —(CH₂)₀₋₂C₃₋₅ cycloalkyl, halogen, —(CH₂)₀₋₃OR^{a}, —CN, —CO₂R^{b}, —(CH₂)₀₋₂NR^{b}SO₂R^{c}, —CF₃, —CH₂CF₃, —OCF₃, or —OCH₂CF₃, wherein R^{a}, R^{b} and R^{c} are —H, —CH₃, or —CH₂CH₃, then both R³ substituents are not methyl. In another subclass of this class, R³ is independently selected from the group consisting of: hydrogen, —OH, —C₂₋₈alkyl, —OC₁₋₈alkyl, halogen, —N(R⁵)₂, —SR⁵, and —CF₃, wherein two C₁₋₈alkyl substituents along with the atoms to which they are attached may form a 4- to 8-membered cycloalkyl or heterocycloalkyl ring. In another subclass of this class, R³ is independently selected from the group consisting of: C₁₋₆ alkyl, and hydrogen, wherein two C₁₋₆alkyl substituents along with the atoms to which they are attached can form a 4- to 8-membered cycloalkyl or heterocycloalkyl ring, and provided that when Z is —N—, Y is H or —OH, X is phenyl substituted with one to three R⁴ substituents and at least one R⁴ is —C₁₋₄alkyl, —(CH₂)₀₋₂C₃₋₅ cycloalkyl, halogen, —(CH₂)₀₋₃OR^{a}, —CN, —CO₂R^{b}, —(CH₂)₀₋₂NR^{b}SO₂R^{c}, —CF₃, —CH₂CF₃, —OCF₃, or —OCH₂CF₃, wherein R^{a}, R^{b} and R^{c} are —H, —CH₃, and —CH₂CH₃, then both R³ substituents are not methyl. In another subclass of this class, R³ methyl. In another subclass of this class, R³ is hydrogen.

In another class of the embodiments, each R³ is independently selected from the group consisting of: hydrogen, —OH, —C₁₋₈alkyl, —OC₁₋₈alkyl, halogen, —N(R⁵)₂, —SR⁵, and —CF₃, and provided that when Z is —N—, Y is H or —OH, X is phenyl substituted with one to three R⁴ substituents and at least one R⁴ is —C₁₋₄alkyl, —(CH₂)₀₋₂C₃₋₅ cycloalkyl, halogen, —(CH₂)₀₋₃OR^{a}, CN, CO₂R^{b}, —(CH₂)₀₋₂NR^{b}SO₂R^{c}, CF₃, CH₂CF₃, OCF₃, or OCH₂CF₃, wherein R^{a}, R^{b} and R^{c} are —H, —CH₃, or —CH₂CH₃, then both R³ substituents are not methyl. In a subclass of this class, each R³ is independently selected from the group consisting of: hydrogen, —OH, —C₁₋₈alkyl, —OC₁₋₈alkyl, halogen, —N(R⁵)₂, —SR⁵, and —CF₃, and provided that when Z is —N—, Y is H or —OH, X is phenyl substituted with one to three R⁴ substituents and at least one R⁴ is —C₁₋₄alkyl, —(CH₂)₀₋₂C₃₋₅ cycloalkyl, halogen, —(CH₂)₀₋₃OR^{a}, CN, CO₂R^{b}, —(CH₂)₀₋₂NR^{b}SO₂R^{c}, CF₃, CH₂CF₃, OCF₃, or OCH₂CF₃, wherein R^{a}, R^{b} and R^{c} are —H, —CH₃, or —CH₂CH₃, then both R³ substituents are not methyl. In another subclass of this class, R³ is independently selected from the group consisting of: hydrogen, —OH, —C₂₋₈alkyl, —OC₁₋₈alkyl, halogen, —N(R⁵)₂, —SR⁵, and —CF₃. In another subclass of this class, R³ is independently selected from the group consisting of: C₁₋₆ alkyl, and hydrogen, wherein two C₁₋₆alkyl substituents along with the atoms to which they are attached can form a 4- to 8-membered cycloalkyl or heterocycloalkyl ring, and provided that when Z is —N—, Y is H or —OH, X is phenyl substituted with one to three R⁴ substituents and at least one R⁴ is —C₁₋₄alkyl, —(CH₂)₀₋₂C₃₋₅ cycloalkyl, halogen, —(CH₂)₀₋₃OR^{a}, CN, CO₂R^{b}, —(CH₂)₀₋₂NR^{b}SO₂R^{c}, CF₃, CH₂CF₃, OCF₃, or OCH₂CF₃, wherein R^{a}, R^{b} and R^{c} are —H, —CH₃, and —CH₂CH₃, then both R³ substituents are not methyl. In another subclass of this class, R³ methyl. In another subclass of this class, R³ is hydrogen.

In a class of the embodiments, each R⁴ is independently selected from the group consisting of: —C₁₋₈ alkyl, —C₂₋₈ alkenyl, —(CH₂)ₙ-phenyl, —(CH₂)ₙ-naphthyl, —(CH₂)ₙ-heteroaryl, —(CH₂)ₙC₂₋₇ heterocycloalkyl, —(CH₂)ₙC₃₋₇ cycloalkyl, —(CH₂)ₙ-halogen, —(CH₂)ₙ—OR⁶, —(CH₂)ₙ—OSi(C₁₋₆alkyl)₃, —(CH₂)ₙC(O)R⁶, —(CH₂)ₙOC(O)R⁶, —(CH₂)ₙC(O)OR⁶, —(CH₂)ₙC≡N, NO₂, —(CH₂)ₙN(R⁶)₂, —(CH₂)ₙC(O)N(R⁶)₂, —(CH₂)ₙNR⁶C(O)R⁶, —(CH₂)ₙNR⁶C(O)OR⁶, —(CH₂)ₙNR⁶C(O)-heteroaryl, —(CH₂)ₙNR⁶C(O)N(R⁶)₂, —(CH₂)ₙC(O)NR⁶N(R⁶)₂, —(CH₂)ₙC(O)NR⁶NR⁶C(O)R⁶, —(CH₂)ₙNR⁶S(O)ₚR⁶, —(CH₂)ₙS(O)ₚN(R⁶)₂, —(CH₂)ₙS(O)ₚR⁶, —O(CH₂)ₙC(O)N(R⁶)₂, —CF₃, —CH₂CF₃, —OCF₃, and —OCH₂CF₃, wherein alkenyl, phenyl, naphthyl, heteroaryl are unsubstituted or substituted with one to three substituents independently selected from halogen, hydroxy, —C₁₋₆alkyl, trifluoromethyl, —C₁₋₆alkoxy, —CO₂C₁₋₆alkyl, and —CO₂H, and wherein any alkyl, cycloalkyl, heterocycloalkyl, and (CH₂) carbon atom in R⁴ is unsubstituted or substituted with one to two groups independently selected from halogen, hydroxy, oxo, —C₁₋₆alkyl, trifluoromethyl, —C₁₋₆alkoxy, —CO₂C₁₋₆alkyl, and —CO₂H, or two R⁴ substituents on the same carbon atom are taken together with the carbon atom to form a cyclopropyl group. In a subclass of this class, each R⁴ is independently selected from the group consisting of: —C₁₋₈ alkyl, —(CH₂)ₙ-heteroaryl, —(CH₂)ₙ-halogen, —(CH₂)ₙNR⁶C(O)R⁶, and —(CH₂)ₙNR⁶S(O)ₚR⁶, wherein heteroaryl are unsubstituted or substituted with one to three substituents independently selected from halogen, hydroxy, —C₁₋₆alkyl, trifluoromethyl, —C₁₋₆alkoxy, —CO₂C₁₋₆alkyl, and —CO₂H, and wherein any alkyl and (CH₂) carbon atom in R⁴ is unsubstituted or substituted with one to two groups independently selected from halogen, hydroxy, oxo, —C₁₋₆alkyl, trifluoromethyl, —C₁₋₆alkoxy, and —CO₂C₁₋₆alkyl. In another subclass of this class, each R⁴ is independently selected from the group consisting of: —CH₃, —CH(CH₃)-tetrazole, —CH(CH₃)-triazole, —C(CH₃)₂-triazole, Cl, F, —CH₂NHC(O)CH₃, —CH(CH₂CH₃)NHC(O)CH₃, —CH₂C(CH₃)₂NHC(O)CH₃, —N(CH₂CH(CH₂F)₂)SO₂CH₃, —N(CH₂CH(CH₃)₂)SO₂CH₃, —N(CH₂CF₃)SO₂CH₃, —N(CH₂CF₃)SO₂-cyclopropyl, and —N(CH₂-cyclopropyl)SO₂CH₃, wherein the triazole and tetrazolel are unsubstituted or substituted with one to three substituents independently selected from halogen, hydroxy, —C₁₋₆alkyl, trifluoromethyl, —C₁₋₆alkoxy, —CO₂C₁₋₆alkyl, and —CO₂H, and wherein any alkyl and (CH₂) carbon atom in R⁴ is unsubstituted or substituted with one to two groups independently selected from halogen, hydroxy, oxo, —C₁₋₆alkyl, trifluoromethyl, —C₁₋₆alkoxy, and —CO₂C₁₋₆alkyl. In another subclass of this class, each R⁴ is independently selected from the group consisting of: —CH₃, —CH(CH₃)-tetrazole, —CH(CH₃)-triazole, —C(CH₃)₂-triazole, Cl, F, —CH(CH₂CH₃)NHC(O)CH₃, —N(CH₂CHF₂)SO₂CH₃, —N(CH₂CH(CH₃)₂)SO₂CH₃, —N(CH₂CF₃)SO₂CH₃, —N(CH₂CF₃)SO₂-cyclopropyl, and —N(CH₂-cyclopropyl)SO₂CH₃, wherein the triazole, tetrazole and any methylene (CH₂) carbon atom in R⁴ are unsubstituted or substituted with one to three substituents independently selected from —C₁₋₆alkyl.

In another class of the embodiments, R⁵ is independently selected from the group consisting of: hydrogen, —C₁₋₈alkyl, —C₂₋₈alkenyl, —C₂₋₈alkynyl, —C₁₋₈alkoxy, —(CH₂)ₙC₃₋₇cycloalkyl, —(CH₂)ₙC₂₋₇heterocycloalkyl, —(CH₂)ₙ-phenyl, —(CH₂)ₙ-naphthyl, —(CH₂)ₙ-heteroaryl, and —(CH₂)ₙC₃₋₇bicycloalkyl, wherein alkenyl, alkynyl, phenyl, naphthyl, and heteroaryl are unsubstituted or substituted with one to three groups independently selected from R⁴, and alkyl, alkoxy, cycloalkyl, heterocycloalkyl, and bicycloalkyl are unsubstituted or substituted with one to three groups independently selected from R⁴ and oxo, and wherein any methylene (CH₂) in R⁵ is unsubstituted or substituted with one to two groups independently selected from halogen, hydroxy, and —C₁₋₆alkyl, or two R⁵ groups together with the atom to which they are attached form a 5- to 8-membered mono- or bicyclic ring system optionally containing an additional heteroatom selected from O, S, and —NC₁₋₄ alkyl. In a subclass of this class, each R⁵ is independently selected from the group consisting of: hydrogen, and —C₁₋₈alkyl, wherein alkyl is unsubstituted or substituted with one to three groups independently selected from R⁴.

In another class of the embodiments, each R⁶ is independently selected from the group consisting of: hydrogen, —C₁₋₆ alkyl, —(CH₂)ₙ-phenyl, —(CH₂)ₙ-heteroaryl, —(CH₂)ₙ-naphthyl, —(CH₂)ₙ-heterocycloalkyl, —(CH₂)ₙC₃₋₇cycloalkyl, —(CH₂)ₙC₃₋₇bicycloalkyl, —(CH₂)ₙCF₃, and —(CH₂)ₙCHF₂;

wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy; or two R⁶ groups together with the atom to which they are attached form a 4- to 8-membered mono- or bicyclic ring system optionally containing an additional heteroatom selected from O, S, and NC₁₋₄ alkyl. In a subclass of this class, each R⁶ is independently selected from the group consisting of: hydrogen, —C₁₋₆ alkyl, —(CH₂)ₙC₃₋₇cycloalkyl, and —(CH₂)ₙCF₃, wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy; or two R⁶ groups together with the atom to which they are attached form a 4- to 8-membered mono- or bicyclic ring system optionally containing an additional heteroatom selected from O, S, and NC₁₋₄ alkyl. In another subclass of this class, each R⁶ is independently selected from the group consisting of: hydrogen, —CH₃, —CH₂-cyclopropyl, -cyclopropyl, —CH₂CF₃, —CH₂CHF₂, and —CH₂CH(CH₃)₂, wherein the alkyl and cycloalkyl groups are unsubstituted or substituted with one to three groups independently selected from halogen, —C₁₋₆alkyl, hydroxy, and C₁₋₄ alkoxy.

In another class of the embodiments, each R⁷ is independently selected from the group consisting of: hydrogen, and —C₁₋₈ alkyl, wherein alkyl is unsubstituted or substituted with one to three groups independently selected from halogen, —C₁₋₆alkyl, hydroxy, and C₁₋₄ alkoxy. In a subclass of this class, each R⁷ is hydrogen. In another subclass of this class, R⁷ is —C₁₋₈alkyl, wherein alkyl is unsubstituted or substituted with one to three groups independently selected from halogen, —C₁₋₆alkyl, hydroxy, and C₁₋₄ alkoxy.

In another class of the embodiments, each R⁸ is independently selected from the group consisting of: —C₁₋₆alkyl, —(CH₂)ₙphenyl, —(CH₂)ₙnaphthyl, —(CH₂)ₙheteroaryl, —(CH₂)ₙC₂₋₇heterocycloalkyl, —(CH₂)ₙC₃₋₇cycloalkyl, halogen, —OR⁶, —(CH₂)ₙN(R⁶)₂, —(CH₂)ₙC≡N, —(CH₂)ₙCO₂R⁶, —NO₂, —(CH₂)ₙNR⁶S(O)ₚR⁶, —(CH₂)ₙS(O)ₚN(R⁶)₂, —(CH₂)ₙS(O)ₚR⁶, —(CH₂)ₙNR⁶C(O)N(R⁶)₂, —(CH₂)ₙC(O)N(R⁶)₂, —(CH₂)ₙNR⁶C(O)R⁶, —(CH₂)ₙNR⁶CO₂R⁶, —(CH₂)ₙNR⁶C(O)-heteroaryl, —(CH₂)ₙC(O)NR⁶N(R⁶)₂, —(CH₂)ₙC(O)NR⁶NR⁶C(O)R⁶, —O(CH₂)ₙC(O)N(R⁶)₂, —CF₃, —CH₂CF₃, —OCF₃, and —OCH₂CF₃, wherein phenyl, naphthyl, and heteroaryl are unsubstituted or substituted with one to three substituents independently selected from halogen, hydroxy, —C₁₋₆alkyl, trifluoromethyl, and C₁₋₄ alkoxy, and wherein alkyl, cycloalkyl, heterocycloalkyl, and (CH₂) are unsubstituted or substituted with one to three substituents independently selected from halogen, hydroxy, oxo, —C₁₋₆alkyl, trifluoromethyl, and C₁₋₄ alkoxy. In a subclass of this class, R⁸ is independently selected from the group consisting of: C₁₋₆ alkyl, -heteroaryl, halogen, OR⁵, NO₂, —SR⁵, and CF₃. In another subclass of this class,

R⁸ is independently selected from the group consisting of: C₁₋₆ alkyl, and halogen. In a subclass of this subclass, R⁸ is halogen. In another subclass of this subclass, R⁸ is fluoro or chloro. In another subclass of this subclass, R⁸ is fluoro.

In another class of these embodiments, each R⁹ is independently selected from the group consisting of: —(CH₂)ₙ-halogen, —C₁₋₆alkyl, —(CH₂)ₙ—CO₂R⁶, —(CH₂)ₙ—OR⁶, —(CH₂)ₙ-phenyl, —(CH₂)ₙ-heteroaryl, —(CH₂)ₙ-naphthyl, —(CH₂)ₙ-heterocycloalkyl, —(CH₂)ₙC₃₋₇cycloalkyl, —(CH₂)ₙC₃₋₇bicycloalkyl, —(CH₂)ₙCF₃, and —(CH₂)ₙCHF₂, wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy; or two R⁶ groups together with the atom to which they are attached form a 4- to 8-membered mono- or bicyclic ring system optionally containing an additional heteroatom selected from O, S, and NC₁₋₄ alkyl. In a subclass of this class, each R⁹ is independently selected from the group consisting of: —(CH₂)ₙ-halogen, —C₁₋₆alkyl, —(CH₂)ₙ—CO₂R⁶, and —(CH₂)ₙ—OR⁶, wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy; or two R⁶ groups together with the atom to which they are attached form a 4- to 8-membered mono- or bicyclic ring system optionally containing an additional heteroatom selected from O, S, and NC₁₋₄ alkyl. In another subclass of this class, each R⁹ is independently selected from the group consisting of: F, —CH₂F, —CH₃, —CH₂CH₂CH₃, —CO₂H, —OH, —OCH₃, —CH₂OH, and —CH₂OCH₃, wherein the alkyl, phenyl, groups are unsubstituted or substituted with one to three groups independently selected from halogen, —C₁₋₆alkyl, hydroxy, and C₁₋₄ alkoxy.

In another class of the embodiments of the present invention, n is 0, 1, 2 or 3. In a subclass of this class, n is 0. In another subclass of this class, n is 1. In another subclass of this class, n is 2. In another subclass of this class, n is 3.

In another class of the embodiments, r is 1 or 2. In a subclass of this class, r is 1. In another subclass of this class, r is 2. In another subclass of this class, r is 1 and s is 1. In another class of the embodiments of the present invention, r is 2 and s is 1.

In another class of the embodiments, s is 0, 1 or 2. In a subclass of this class, s is 0. In another subclass of this class, s is 1. In another subclass of this class, s is 2.

In another class of the embodiments, p is 0, 1, or 2. In a subclass of this class, p is 0. In another subclass of this class, p is 1. In another subclass of this class, p is 2.

In another class of the embodiments, q is 0, 1, 2, 3 or 4. In a subclass of this class, q is 1, 2, 3 or 4. In another subclass of this class, q is 1. In another subclass of this class, q is 2. In another subclass of this class, q is 3. In another subclass of this class, q is 4. In another subclass q is 2 or 3.

Illustrative but nonlimiting examples of compounds of the present invention that are useful as melanocortin-4 receptor agonists are the following:

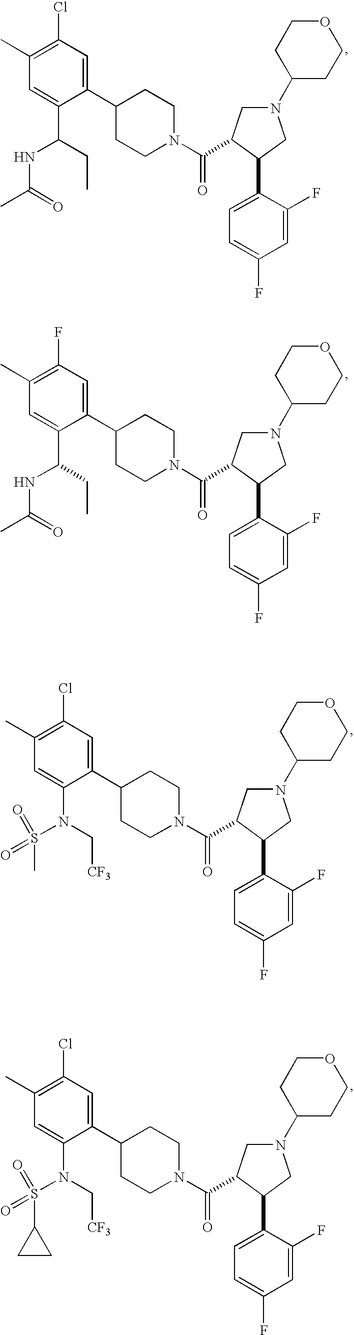

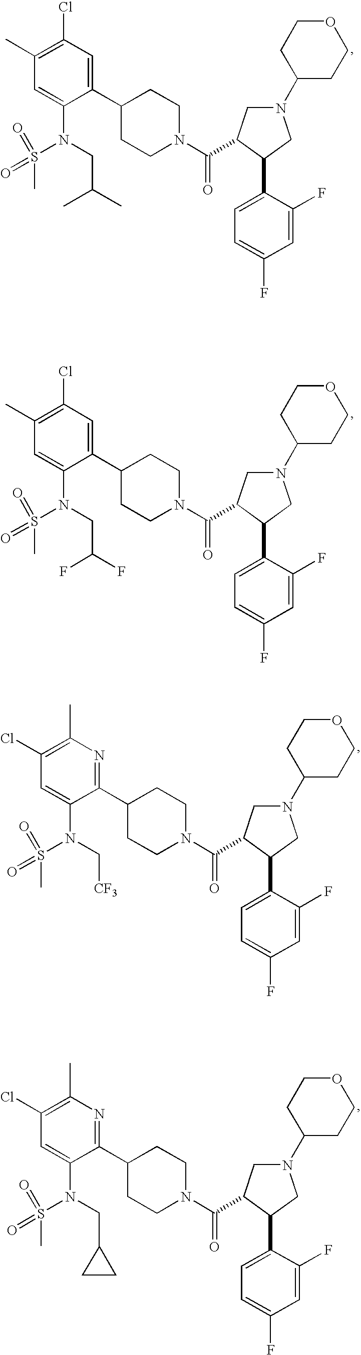

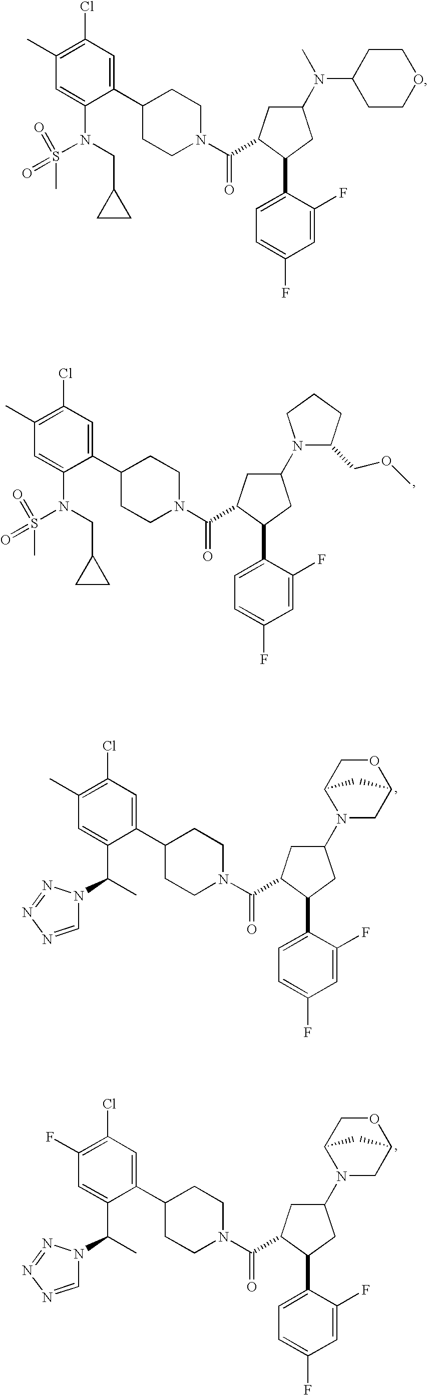

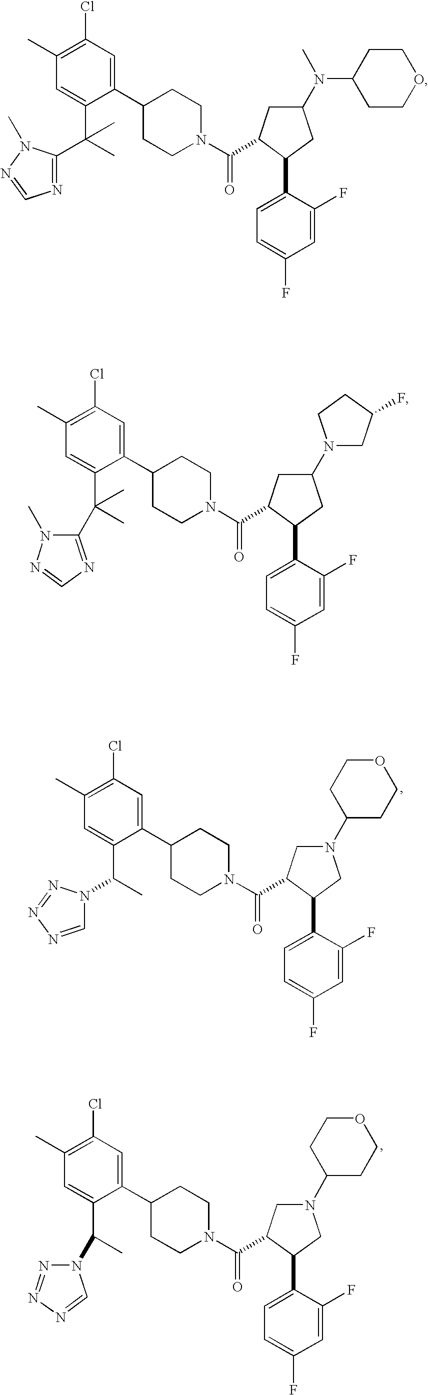

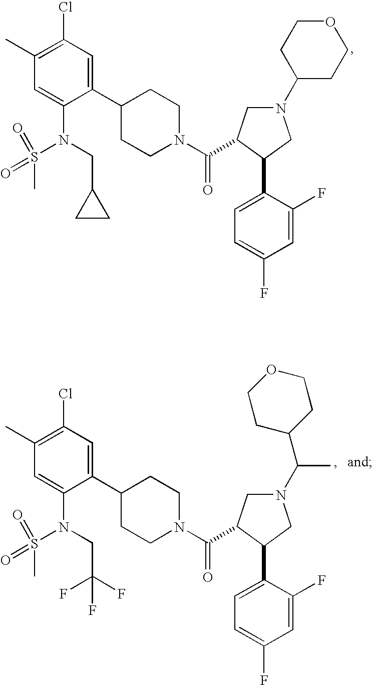

or a pharmaceutically acceptable salt thereof.

The compounds of structural formula I are effective as melanocortin receptor ligands and are particularly effective as selective ligands of the melanocortin-4 receptor. They are therefore useful for the treatment and/or prevention of disorders responsive to the modulation of the melanocortin-4 receptor, such as obesity, diabetes, obesity-related disorders, nicotine addiction, alcoholism, as well as male and female sexual dysfunction, and in particular male erectile dysfunction, cachexia, wasting, anorexia and weight loss.

More particularly, the selective melanocortin-4 receptor (MC-4R) agonists of formula I are useful for the treatment of disorders responsive to the activation of the melancortin-4 receptor, such as obesity, diabetes, nicotine addiction, alcoholism, male sexual dysfunction, and female sexual dysfunction. Another aspect of the present invention provides a method for the treatment or prevention of disorders, diseases or conditions responsive to the modulation of the melanocortin-4 receptor in a subject in need thereof which comprises administering to the subject a therapeutically or prophylactically effective amount of a compound of formula I, II, III, IV or V, or a pharmaceutically acceptable salt thereof.

Furthermore, the selective melanocortin-4 receptor (MC-4R) antagonists of formula I are useful for the treatment of disorders responsive to the deactivation of the melanocortin-4 receptor, such as cachexia, wasting, anorexia, frailty, sarcopenia and weight loss.

Another aspect of the present invention provides a method for the treatment or prevention of obesity, diabetes, or an obesity related disorder in a subject in need thereof which comprises administering to said subject a therapeutically or prophylactically effective amount of a melanocortin-4 receptor agonist of the present invention. Another aspect of the present invention provides a method for the treatment or prevention of obesity in a subject in need thereof which comprises administering to the subject a therapeutically or prophylactically effective amount of a compound of formula I, II, III, IV or V, or a pharmaceutically acceptable salt thereof. Another aspect of the present invention provides a method for the treatment or prevention of diabetes mellitus in a subject in need thereof comprising administering to the subject a therapeutically or prophylactically effective amount of a compound of formula I, II, III, IV or V, or a pharmaceutically acceptable salt thereof. Another aspect of the present invention provides a method for the treatment or prevention of an obesity-related disorder selected from the group consisting of overeating, binge eating, and bulimia, hypertension, elevated plasma insulin concentrations, insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, metabolic syndrome, insulin resistance syndrome, sexual and reproductive dysfunction, infertility, hypogonadism, hirsutism, obesity-related gastro-esophageal reflux, Pickwickian syndrome, cardiovascular disorders, inflammation, systemic inflammation of the vasculature, arteriosclerosis, hypercholesterolemia, hyperuricaemia, lower back pain, gallbladder disease, gout, and kidney cancer, cardiac hypertrophy, left ventricular hypertrophy, nicotine addiction and alcoholism, in a subject in need thereof which comprises administering to the subject a therapeutically or prophylactically effective amount of a compound of formula I, II, III, IV or V, or a pharmaceutically acceptable salt thereof.

The present invention also relates to methods for treating or preventing obesity by administering the melanocortin-4 receptor agonist of the present invention in combination with a therapeutically or prophylactically effective amount of another agent known to be useful to treat or prevent the condition. The present invention also relates to methods for treating or preventing diabetes by administering the melanocortin-4 receptor agonist of the present invention in combination with a therapeutically or prophylactically effective amount of another agent known to be useful to treat or prevent the condition.

Another aspect of the present invention provides a method for the treatment or prevention of female or male sexual dysfunction, including male erectile dysfunction, which comprises administering to a subject in need of such treatment or prevention a therapeutically or prophylactically effective amount of a melanocortin-4 receptor agonist of the present invention. Another aspect of the present invention provides a method for the treatment or prevention of erectile dysfunction in a subject in need thereof comprising administering to the subject a therapeutically or prophylactically effective amount of a compound of formula I, II, III, IV, or V, or a pharmaceutically acceptable salt thereof. The present invention also relates to methods for treating or preventing erectile dysfunction by administering the melanocortin-4 receptor agonist of the present invention in combination with a therapeutically or prophylactically effective amount of another agent known to be useful to treat the condition.

Another aspect of the present invention provides a method for the treatment or prevention of alcoholism which comprises administering to a subject in need of such treatment or prevention a therapeutically or prophylactically effective amount of a melanocortin 4 receptor agonist of the present invention. The present invention also provides a method for reducing alcohol consumption which comprises administering to a subject in need of such treatment or prevention a therapeutically or prophylactically effective amount of a melanocortin 4 receptor agonist of the present invention.

Another aspect of the present invention provides a method for the treatment or prevention of nicotine addiction which comprises administering to a subject in need of such treatment or prevention a therapeutically or prophylactically effective amount of a melanocortin 4 receptor agonist of the present invention. The present invention also provides a method for reducing nicotine consumption which comprises administering to a subject in need of such treatment a therapeutically effective amount of a melanocortin 4 receptor agonist of the present invention. Yet another aspect of the present invention provides a method for the treatment or prevention of substance addiction which comprises administering to a subject in need of such treatment or prevention a therapeutically or prophylactically effective amount of a melanocortin 4 receptor agonist of the present invention.

Yet another aspect of the present invention provides a method for the treatment or prevention of cachexia which comprises administering to a subject in need of such treatment or prevention a therapeutically or prophylactically effective amount of a melanocortin 4 receptor antagonist of the present invention. The present invention also provides a method for the treatment or prevention of anorexia, wasting or weight loss which comprises administering to a subject in need of such treatment or prevention a therapeutically or prophylactically effective amount of a melanocortin 4 receptor antagonist of the present invention. The present invention further provides a method for the treatment or prevention of anxiety, depression, pain, or neuropathic pain, which comprises administering to a subject in need of such treatment or prevention a therapeutically or prophylactically effective amount of a melanocortin 4 receptor antagonist of the present invention.

Another aspect of the present invention provides a pharmaceutical composition comprising a compound of structural formula I and a pharmaceutically acceptable carrier.

Yet another aspect of the present invention relates to the use of a compound of structural formula I for the manufacture of a medicament useful for the treatment or prevention, or suppression of a disease mediated by the melanocortin-4 receptor in a subject in need thereof.

Yet another aspect of the present invention relates to the use of a melanocortin-4 agonist of the present invention for the manufacture of a medicament useful for the treatment or prevention, or suppression of a disease mediated by the melanocortin-4 receptor, wherein the disease is selected from the group consisting of obesity, diabetes and an obesity-related disorder in a subject in need thereof.

Yet another aspect of the present invention relates to the use of a melanocortin-4 agonist of the present invention for the manufacture of a medicament useful for the treatment or prevention, or suppression of male and female sexual dysfunction, and male erectile dysfunction in a subject in need thereof.

Yet another aspect of the present invention relates to the use of a selective melanocortin-4 agonist of the present invention in the preparation of a medicament useful for treating or preventing alcoholism in a subject in need thereof. The present invention also relates to the use of a selective melanocortin-4 agonist of the present invention in the preparation of a medicament useful for reducing alcohol consumption in a subject in need thereof.

Yet another aspect of the present invention relates to the use of a selective melanocortin 4 receptor agonist of the present invention in the preparation of a medicament useful to treat or prevent nicotine addiction in a subject in need thereof. The present invention also relates to the use of a selective melanocortin 4 receptor agonist of the present invention in the preparation of a medicament useful to reduce nicotine consumption in a subject in need thereof.

Yet another aspect of the present invention relates to the use of a selective melanocortin 4 receptor agonist of the present invention in the preparation of a medicament useful to treat substance addiction in a subject in need thereof.

Yet another aspect of the present invention relates to the use of a selective melanocortin 4 receptor antagonist of the present invention in the preparation of a medicament useful treat or prevent cachexia in a subject in need thereof. The present invention also relates to the use of a selective melanocortin 4 receptor antagonist of the present invention in the preparation of a medicament useful treat or prevent anorexia, wasting, frailty, sarcopenia, or weight loss in a subject in need thereof.

Yet another aspect of the present invention relates to the use of a therapeutically effective amount of a melanocortin-4 receptor agonist of formula I, or a pharmaceutically acceptable salt thereof, and a therapeutically effective amount of an agent selected from the group consisting of an insulin sensitizer, an insulin mimetic, a sulfonylurea, an α-glucosidase inhibitor, a HMG-CoA reductase inhibitor, a serotonergic agent, a ⊖3-adrenoreceptor agonist, a neuropeptide Y1 antagonist, a neuropeptide Y2 agonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a NK-1 antagonist, or a pharmaceutically acceptable salt thereof, for the manufacture of a medicament useful for the treatment, control, or prevention of obesity, diabetes or an obesity-related disorder in a subject in need of such treatment. Yet another aspect of the present invention relates to the use of a therapeutically effective amount of a melanocortin-4 receptor agonist of formula I, and pharmaceutically acceptable salts and esters thereof, and a therapeutically effective amount of an agent selected from the group consisting of an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y2 agonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a NK-1 antagonist, or a pharmaceutically acceptable salt thereof, for the manufacture of a medicament for treatment or prevention of obesity, diabetes or an obesity-related disorder which comprises an effective amount of a melanocortin-4 receptor agonist of formula I and an effective amount of the agent, together or separately. Yet another aspect of the present invention relates to a product containing a therapeutically effective amount of a melanocortin-4 receptor agonist of formula I, or a pharmaceutically acceptable salt thereof; and a therapeutically effective amount of an agent selected from the group consisting of an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y2 agonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a NK-1 antagonist, or a pharmaceutically acceptable salt thereof, as a combined preparation for simultaneous, separate or sequential use in obesity, diabetes, or an obesity-related disorder.

Yet another aspect of the present invention relates to the use of a therapeutically effective amount of a melanocortin-4 receptor agonist of formula I, or a pharmaceutically acceptable salt thereof, and a therapeutically effective amount of an agent selected from the group consisting of: a type V cyclic-GMP-selective phosphodiesterase inhibitor, an α₂-adrenergic receptor antagonist, and a dopaminergic agent, or a pharmaceutically acceptable salt thereof, for the manufacture of a medicament useful for the treatment, control, or prevention of male erectile dysfunction in a subject in need of such treatment. Yet another aspect of the present invention relates to the use of a therapeutically effective amount of a melanocortin-4 receptor agonist of formula I, or a pharmaceutically acceptable salt thereof; and a therapeutically effective amount of an agent selected from the group consisting of a type V cyclic-GMP-selective phosphodiesterase inhibitor, an α₂-adrenergic receptor antagonist, and a dopaminergic agent, and pharmaceutically acceptable salts and esters thereof, for the manufacture of a medicament for treatment or prevention of male erectile dysfunction which comprises an effective amount of a compound of formula I and an effective amount of the agent, together or separately. Yet another aspect of the present invention relates to a product containing a therapeutically effective amount of a melanocortin-4 receptor agonist of formula I, or a pharmaceutically acceptable salt thereof, and a therapeutically effective amount of an agent selected from the group consisting of a type V cyclic-GMP-selective phosphodiesterase inhibitor, an α₂-adrenergic receptor antagonist, and a dopaminergic agent, and pharmaceutically acceptable salts and esters thereof; as a combined preparation for simultaneous, separate or sequential use in male erectile dysfunction.

Melanocortin receptor agonist compounds can be provided in kit. Such a kit typically contains an active compound in dosage forms for administration. A dosage form contains a sufficient amount of active compound such that a beneficial effect can be obtained when administered to a patient during regular intervals, such as 1, 2, 3, 4, 5 or 6 times a day, during the course of 1 or more days. Preferably, a kit contains instructions indicating the use of the dosage form for weight reduction (e.g., to treat obesity) and the amount of dosage form to be taken over a specified time period.

Throughout the instant application, the following terms have the indicated meanings:

The term “alkyl”, as well as other groups having the prefix “alk”, such as alkoxy, alkanoyl, means carbon chains of the designated length which may be in a straight or branched configuration, or combinations thereof. Examples of alkyl groups include, but are not limited to, methyl, ethyl, n-propyl, isopropyl, n-butyl, 1-methylpropyl, 2-methylpropyl, tert-butyl, n-pentyl, 1-methylbutyl, 2-methylbutyl, 3-methylbutyl, 1,2-dimethylpropyl, 1,1-dimethylpropyl, 2,2-dimethylpropyl, n-hexyl, 1-methylpentyl, 2-methylpentyl, 3-methylpentyl, 4-methylpentyl, 1-ethylbutyl, 2-ethylbutyl, 3-ethylbutyl, 1,1-dimethyl butyl, 1,2-dimethylbutyl, 1,3-dimethylbutyl, 2,2-dimethylbutyl, 2,3-dimethylbutyl, 3,3-dimethyl butyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, 4-ethylpentyl, neopentyl, 1-propylbutyl, 2-propylbutyl, 3-propylbutyl, 1,1-dimethylpentyl, 1,2-dimethylpentyl, 1,3-dimethylpentyl, 1,4-dimethylpentyl, 2,2-dimethylpentyl, 2,3-dimethylpentyl. 2,4-dimethylpentyl, 3,3-dimethylpentyl, 3,4-dimethylpentyl, 4,4-dimethylpentyl, 1-methyl-1-ethylbutyl, 1-methyl-2-ethylbutyl, 2-methyl-2-ethylbutyl, 1-ethyl-2-methylbutyl, 1-ethyl-3-methylbutyl, 1,1-diethylpropyl, n-octyl, n-nonyl, and the like. The term “alkyl” also includes methylene (—CH₂).

The term “alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

The term “alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

The term “halogen” includes fluorine, chlorine, bromine and iodine.

The term “C₁₋₄ alkyliminoyl” means C₁₋₃C(═NH)—.

The term “aryl” includes mono- or bicyclic aromatic rings containing only carbon atoms. Examples of aryl include phenyl and naphthyl.

The term “heteroaryl” includes mono- and bicyclic aromatic rings containing from 1 to 4 heteroatoms selected from nitrogen, oxygen and sulfur. Examples thereof include, but are not limited to, pyridyl, furyl, thienyl, pyrrolyl, oxazolyl, thiazolyl, triazolyl, triazinyl, tetrazolyl, thiadiazolyl, imidazolyl, isoxazolyl, isothiazolyl, oxadiazolyl, pyrazolyl, pyrimidinyl, pyrazinyl, pyridazinyl, quinolyl, isoquinolyl, benzimidazolyl, benzofuryl, benzothienyl, indolyl, benzthiazolyl, benzoxazolyl, and the like. In one embodiment of the present invention, heteroaryl is selected from the group consisting of pyridyl, furyl, thienyl, pyrrolyl, oxazolyl, thiazolyl, triazolyl, triazinyl, tetrazolyl, thiadiazolyl, imidazolyl, pyrazolyl, isoxazolyl, isothiazolyl, oxathiazolyl, pyrimidinyl, pyrazinyl, pyridazinyl, quinolyl, isoquinolyl, benzimidazolyl, benzofuryl, benzothienyl, indolyl, benzthiazolyl, and benzoxazolyl. Bicyclic heteroaromatic rings include, but are not limited to, benzothiadiazole, indole, benzothiophene, benzofuran, benzimidazole, benzisoxazole, benzothiazole, quinoline, quinazoline, benzotriazole, benzoxazole, isoquinoline, purine, furopyridine, thienopyridine, benzisodiazole, triazolopyrimidine, and 5,6,7,8-tetrahydroquinoline.

The term “cycloalkyl” includes mono- or bicyclic non-aromatic rings containing only carbon atoms. Examples of cycloalkyl include, but are not limited to, cyclopropyl, cyclobutyl, cyclopentyl, cyclohexyl, cycloheptyl.

The term “heterocycloalkyl” is intended to include mono- and bicyclic ring systems containing containing at least one non-aromatic heterocyclic ring that contains one to four heteroatoms selected from nitrogen, oxygen and sulfur, and in which the non-aromatic heterocyclic ring may be fused to an aryl or heteroaryl ring. Examples of heterocycloalkyls include, but are not limited to, azetidine, piperidine, morpholine, thiamorpholine, pyrrolidine, imidazolidine, tetrahydrofuran, piperazine, 1-thia-4-aza-cyclohexane, tetrahydropyran, azabicycloheptane, azabicyclohexane, 4,5,6,7-tetrahydro-1H-pyrazolo[4,3-c]pyridine; 4,5,6,7-tetrahydroisoxazolo[4,5-c]pyridine-3(2H)-one; and 4,5,6,7-tetrahydrothieno[2,3-c]pyridine.

The term “bridgedC₂₋₇heterocycloalkyl” is a heterocycloalkyl ring in which two ring atoms are connected by a 1-3 carbon methylene bridge, which may be substituted with 1-2 R⁶, includes, but not limited to, the following ring systems: 2,5-diazabicyclo[2.2.1]heptane, 7-azabicyclo[2.2.1]heptane, 2-azabicyclo[2.2.1]heptane, and 2-oxa-5-azabicyclo[2.2,1]heptane.

Certain of the above defined terms may occur more than once in the above formula and upon such occurrence each term shall be defined independently of the other; thus for example, NR⁵R⁵ may represent NH₂, NHCH₃, N(CH₃)CH₂CH₃, and the like.

The term “subject” means a mammal. One embodiment of the term “mammal” is a “human,” said human being either male or female. The instant compounds are also useful for treating or preventing obesity and obesity related disorders in cats and dogs. As such, the term “mammal” includes companion animals such as cats and dogs. The term “mammal in need thereof” refers to a mammal who is in need of treatment or prophylaxis as determined by a researcher, veterinarian, medical doctor or other clinician.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

By a melanocortin receptor “agonist” is meant an endogenous or drug substance or compound that can interact with a melanocortin receptor and initiate a pharmacological or biochemical response characteristic of melanocortin receptor activation. By a melanocortin receptor “antagonist” is meant a drug or a compound that inhibits the melanocortin receptor-associated responses induced by an agonist. The “agonistic” and “antagonistic” properties of the compounds of the present invention were measured in the functional assay described below. The functional assay discriminates a melanocortin receptor agonist from a melanocortin receptor antagonist.

By “binding affinity” is meant the ability of a compound/drug to bind to its biological target, in the present instance, the ability of a compound of structural formula I to bind to a melanocortin receptor. Binding affinities for the compounds of the present invention were measured in the binding assay described below and are expressed as IC₅₀'s.

“Efficacy” describes the relative intensity of response which different agonists produce even when they occupy the same number of receptors and with the same affinity. Efficacy is the property that describes the magnitude of response. Properties of compounds can be categorized into two groups, those which cause them to associate with the receptors (binding affinity) and those that produce a stimulus (efficacy). The term “efficacy” is used to characterize the level of maximal responses induced by agonists. Not all agonists of a receptor are capable of inducing identical levels of maximal responses. Maximal response depends on the efficiency of receptor coupling, that is, from the cascade of events, which, from the binding of the drug to the receptor, leads to the desired biological effect.

The functional activities expressed as EC₅₀'s and the “agonist efficacy” for the compounds of the present invention at a particular concentration were measured in the functional assay described below.

Compounds of structural formula I contain one or more asymmetric centers and can thus occur as racemates and racemic mixtures, single enantiomers, diastereomeric mixtures and individual diastereomers. The present invention includes all such isomeric forms of the compounds of structural formula I, including the E and Z geometric isomers of olefinic double bonds. Some of the compounds described herein may exist as tautomers such as keto-enol tautomers. The individual tautomers as well as mixtures thereof are encompassed within the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general formula I, II, III, IV, and V may be obtained by stereospecific synthesis using optically pure starting materials or reagents of known absolute configuration.

It will be understood that the compounds of the present invention include hydrates, solvates, polymorphs, crystalline, hydrated crystalline and amorphous forms of the compounds of the present invention, and pharmaceutically acceptable salts thereof.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, TEA, trimethylamine, tripropylamine, tromethamine, and the like.

When the compound of formula I, II, III, IV or V is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Particularly preferred are citric, fumaric, hydrobromic, hydrochloric, maleic, phosphoric, sulfuric, and tartaric acids.

It will be understood that, as used herein, references to the compounds of Formula I are meant to also include the pharmaceutically acceptable salts, such as the hydrochloride salts.

Compounds of formula I are melanocortin receptor ligands and as such are useful in the treatment, control or prevention of diseases, disorders or conditions responsive to the modulation of one or more of the melanocortin receptors including, but are not limited to, MC-1, MC-2, MC-3, MC-4, or MC-5. In particular, the compounds of formula I act as melanocortin-4 receptor agonists and antagonists useful in the treatment, control or prevention of diseases, disorders or conditions responsive to the activation or deactivation of the melanocortin-4 receptor. Such diseases, disorders or conditions include, but are not limited to, obesity (by reducing appetite, increasing metabolic rate, reducing fat intake or reducing carbohydrate craving), diabetes mellitus (by enhancing glucose tolerance, decreasing insulin resistance), hypertension, hyperlipidemia, osteoarthritis, cancer, gall bladder disease, sleep apnea, depression, anxiety, compulsion, neuroses, insomnia/sleep disorder, substance abuse, pain, male and female sexual dysfunction (including male impotence, loss of libido, female sexual arousal dysfunction, female orgasmic dysfunction, hypoactive sexual desire disorder, sexual pain disorder and male erectile dysfunction), fever, inflammation, immunomodulation, rheumatoid arthritis, skin tanning, acne and other skin disorders, neuroprotective and cognitive and memory enhancement including the treatment of Alzheimer's disease. Some agonists encompassed by formula I show highly selective affinity for the melanocortin-4 receptor (MC-4R) relative to MC-1R, MC-2R, MC-3R, and MC-5R, which makes them especially useful in the prevention and treatment of obesity, female sexual dysfunction, male sexual dysfunction including erectile dysfunction, alcoholism and nicotine addiction. Some antagonists encompassed by formula I show highly selective affinity for the melanocortin-4 receptor (MC-4R) relative to MC-1R, MC-2R, MC-3R, and MC-5R, which makes them especially useful in the prevention and treatment of cachexia, wasting and anorexia.

The compositions of the present invention are useful for the treatment or prevention of disorders associated with excessive food intake, such as obesity and obesity-related disorders. The obesity herein may be due to any cause, whether genetic or environmental.

The obesity-related disorders herein are associated with, caused by, or result from obesity. Examples of obesity-related disorders include overeating, binge eating, and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hyperventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer, nicotine addiction, substance addiction and alcoholism. The compositions of the present invention are also useful for reducing the risk of secondary outcomes of obesity, such as reducing the risk of left ventricular hypertrophy.

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

Treatment of diabetes mellitus refers to the administration of a compound or combination of the present invention to treat diabetes. One outcome of treatment may be decreasing the glucose level in a subject with elevated glucose levels. Another outcome of treatment may be improving glycemic control. Another outcome of treatment may be decreasing insulin levels in a subject with elevated insulin levels. Another outcome of treatment may be decreasing plasma triglycerides in a subject with elevated plasma triglycerides. Another outcome of treatment may be lowering LDL cholesterol in a subject with high LDL cholesterol levels. Another outcome of treatment may be increasing HDL cholesterol in a subject with low HDL cholesterol levels. Another outcome may be decreasing the LDL/HDL ratio in a subject in need thereof. Another outcome of treatment may be increasing insulin sensivity. Another outcome of treatment may be enhancing glucose tolerance in a subject with glucose intolerance. Another outcome of treatment may be decreasing insulin resistance in a subject with increased insulin resistance or elevated levels of insulin. Another outcome may be decreading triglycerides in a subject with elevated triglycerides. Yet another outcome may be improving LDL cholesterol, non-HDL cholesterol, triglyceride, HDL cholesterol or other lipid analyte profiles.

Prevention of diabetes mellitus refers to the administration of a compound or combination of the present invention to prevent the onset of diabetes in a subject at risk thereof.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

Treatment of obesity and obesity-related disorders refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in subjects in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovary disease, cardiovascular diseases, osteoarthritis, dermatological disorders, hypertension, insulin resistance, hypercholesterolemia, hypertriglyceridemia, and cholelithiasis.

“Male sexual dysfunction” includes impotence, loss of libido, and erectile dysfunction.

“Erectile dysfunction” is a disorder involving the failure of a male subject to achieve erection, ejaculation, or both. Symptoms of erectile dysfunction include an inability to achieve or maintain an erection, ejaculatory failure, premature ejaculation, or inability to achieve an orgasm. An increase in erectile dysfunction and sexual dysfunction can have numerous underlying causes, including but not limited to (1) aging, (b) an underlying physical dysfunction, such as trauma, surgery, and peripheral vascular disease, and (3) side-effects resulting from drug treatment, depression, and other CNS disorders.

Treatment of male sexual dysfunction refers to the administration of a compound or combination of the present invention to treat impotence and/or loss of libido, and/or erectile dysfunction in a male subject in need thereof. One outcome of treatment may be a decrease in impotence. Another outcome of treatment may be an increase in libido. Yet another outcome of treatment may be a decrease in the magnitude or frequency of erectile dysfunction. Treatment of male erectile dysfunction refers to the administration of a compound or combination of the present invention to treat one or more of the symptoms of male erectile dysfunction in a male subject in need thereof. One outcome of treatment may be increasing the ability to achieve an erection. Another outcome of treatment may be increasing the ability to maintain an erection. Another outcome of treatment may be reducing ejaculatory failure. Another outcome of treatment may be decreasing premature ejaculation. Yet another outcome of treatment may be increasing the ability to achieve an orgasm. Prevention of male sexual dysfunction and male erectile dysfunction refers to the administration of the compounds or combinations of the present invention to prevent the symptoms of sexual dysfunction and erectile dysfunction in a male subject at risk thereof.

“Female sexual dysfunction” can be seen as resulting from multiple components including dysfunction in desire, sexual arousal, sexual receptivity, and orgasm related to disturbances in the clitoris, vagina, periurethral glans, and other trigger points of sexual function. In particular, anatomic and functional modification of such trigger points may diminish the orgasmic potential in breast cancer and gynecologic cancer patients. Treatment of female sexual dysfunction with an MC-4 receptor agonist can result in improved blood flow, improved lubrication, improved sensation, facilitation of reaching orgasm, reduction in the refractory period between orgasms, and improvements in arousal and desire. In a broader sense, “female sexual dysfunction” also incorporates sexual pain, premature labor, and dysmenorrhea.

The compositions of the present invention are useful for the treatment or prevention of disorders associated with excessive food intake, such as obesity and obesity-related disorders.

“Cachexia” is a wasting disorder that is characterized by weight loss, loss of muscle protein, loss of lean body mass, anorexia, and weakness, and is typically associated with chronic diseases, including cancer cachexia and cachexia associated with AIDS, chronic obstructive pulmonary disease, rheumatiod arthritis, tuberculosis and Crohn's disease. Cancer cachexia is a syndrome of progressive weight loss, anorexia, and persistent erosion of the body in response to a malignant growth; cachexia may be present in early stages of tumor growth before any signs or symptoms of malignancy.

Treatment of cachexia refers to the administration of a compound or combination of the present invention to treat one or more of the symptoms of cachexia in a subject in need thereof.

Prevention of cachexia refers to the administration of the compounds or combinations of the present invention to prevent the symptoms of cachexia or wasting in a subject at risk thereof, including but not limited to, a subject diagnosed with cancer.

The compositions of the present invention are useful for the treatment or prevention of nicotine addiction, substance addiction, and alcoholism, as well as nicotine addiction related disorders, substance abuse related disorders, and alcoholism related disorders.

The term “nicotine” as used herein refers to nicotine contained in tobacco and other naturally occurring sources, as well as synthetic nicotine, and salts thereof, including but not limited to, the salicylate or bitartrate salt thereof. Nicotine addiction is a destructive pattern of nicotine use, leading to significant social occupational, or medical impairment and characterized by three or more of the following symptoms: 1) nicotine tolerance (a need for markedly increased amounts of nicotine to achieve intoxication, or markedly diminished effect with continued use of the same amount of nicotine); 2) nicotine withdrawal symptoms (sweating or rapid pulse, increased hand tremor, insomnia, nausea or vomiting, physical agitation, anxiety, transient visual, tactile, or auditory hallucinations or illusions, grand mal seizures), 3) nicotine administration to relieve or avoid withdrawal symptoms, 4) greater use than nicotine than intended, 5) unsuccessful efforts to cut down or control nicotine use, 6) persistent desire or unsuccessful efforts to cut down or control nicotine use, 7) great deal of time spent using nicotine, 8) nicotine caused reduction in social, occupational or recreational activities, and 9) continued use of nicotine despite knowledge of having a persistent or recurrent physical or psychological problem that is likely to have been worsened by nicotine use. Nicotine addiction-related disorders include, but are not limited to: cancer of the lung, mouth, pharynx, larynx, esophagus, cervix, kidney, ureter and bladder; chronic bronchitis; emphysema; asthma; heart disease, including stroke, heart attack, vascular disease, and aneurysm; premature delivery; spontaneous abortion; and infants with decreased birth weight; as well as nicotine withdrawal symptoms. “Treatment” (of nicotine addiction) refers to the administration of the compounds or combinations of the present invention to reduce or inhibit the use of nicotine by a subject. One outcome of treatment may be reducing the use of nicotine in a subject relative to the subject's nicotine use prior to treatment. Another outcome of treatment may be inhibiting the use of nicotine in a subject. Another outcome of treatment may be decreasing the severity of nicotine intake, such as decreasing the amount of nicotine consumed, in a subject. “Prevention” (of nicotine addiction) refers to the administration of the compounds or combinations of the present invention to prevent nicotine abuse, nicotine addiction or developing a nicotine addiction-related disorder in a subject by administration prior to the start of nicotine use. One outcome of prevention may be to prevent nicotine use in a subject by administration prior to the start of nicotine use. Another outcome of prevention may be to prevent nicotine addiction in a subject. Another outcome of prevention may be to prevent the development of a nicotine addiction related disorder in a subject. Another outcome of prevention may be preventing nicotine use from occurring if the treatment is administered prior to the onset of nicotine use in a subject. Another outcome of prevention may be to administer the compounds or combinations of the present invention to prevent nicotine use in a subject at risk of developing nicotine addiction.

Substance addiction includes opiate addiction, cocaine addiction, marijuana addiction, and amphetamine addiction. The term “opiate” as used herein includes, but is not limited to, heroin; narcotics, such as morphine; opium; codeine; oxycodone (Oxycontin®); propoxyphene (Darvon®); hydrocodone (Vicodin®), hydromorphone (Dilaudid®); meperidine (Demerol®), and Lomotil®. The term “amphetamine(s)” as used herein includes, but is not limited to, amphetamine, dextroamphetamine, and methamphetamine. “Treatment” (of substance addiction) refers to the administration of the compounds or combinations of the present invention to reduce or inhibit the use of the substance by a subject. One outcome of treatment may be reducing the use of the substance in a subject relative to the subject's substance use prior to treatment. Another outcome of treatment may be inhibiting the use of the substance in a subject. Another outcome of treatment may be decreasing the occurrence of substance intake in a subject. Another outcome of treatment may be decreasing the severity of substance intake, such as decreasing the amount of the substance consumed, in a subject. Another outcome of treatment may be to administer the compounds or combinations of the present invention to reduce or inhibit the consumption of the substance in a subject in need thereof. “Prevention” (of substance addiction) refers to the administration of the compounds or combinations of the present invention to prevent substance addiction or developing a substance addiction-related disorder in a subject. One outcome of prevention may be to prevent substance use in a subject by administration prior to the start of substance use. Another outcome of prevention may be to prevent substance addiction in a subject. Another outcome of prevention may be to prevent the development of a substance addiction related disorder in a subject. Another outcome of prevention may be preventing substance use from occurring if the treatment is administered prior to the onset of substance use in a subject.

The compounds of the present invention are useful to inhibit or reduce voluntary alcohol consumption, and for the treatment or prevention of alcoholism, alcohol abuse, and alcohol-related disorders. Alcoholism is a disease that is characterized by abnormal alcohol seeking behavior that leads to impaired control over drinking, and may include some or all of the following symptoms: narrowing of drinking repertoire (drinking only one brand or type of alcoholic beverage); craving (a strong need or urge to drink), loss of control (not being able to stop drinking once drinking has begun), drink seeking behavior (attending only social events that include drinking); physical dependence (withdrawal symptoms, such as nausea, sweating, shakiness, and anxiety after cessation of drinking), drinking to relieve or avoid withdrawal symptoms; and tolerance (the need to drink greater amounts of alcohol to achieve previous effects); subjective awareness of the compulsion to drink or craving for alcohol; and relapse (a return to drinking after a period of abstinence). Alcohol related disorders include, but are not limited to: liver disease, such as hepatitis, inflammation of the liver, and alcoholic cirrhosis; heart disease; high blood pressure; stroke; certain forms of cancer, such as esophageal, mouth, throat, voice box, breast, colon and rectal cancer; pancreatitis; alcoholic dementia, Wernicke-Korsakoff syndrome, brain damage, slow bone healing; impaired wound healing; diminished immune defenses; and death. “Treatment” (of alcoholism) refers to the administration of the compounds or combinations of the present invention to reduce or inhibit the consumption of alcohol in a subject. One outcome of treatment may be reducing the consumption of alcohol in a subject relative to the subject's alcohol consumption prior to treatment. Another outcome of treatment may be inhibiting consumption of alcohol in a subject. Another outcome of treatment may be decreasing the occurrence of alcohol intake in a subject. Another outcome of treatment may be decreasing the severity of alcohol intake, such as decreasing the amount of alcohol consumed, in a subject. Another outcome of treatment may be to administer the compounds or combinations of the present invention to reduce or inhibit the consumption of alcohol in a subject in need thereof. “Prevention” (of alcoholism) refers to the administration of the compounds or combinations of the present invention to prevent alcohol intake, alcohol consumption, alcohol abuse, alcoholism or developing an alcohol-related disorder in a subject. One outcome of prevention may be to prevent alcohol intake in a subject by administration prior to the start of alcohol consumption. Another outcome of prevention may be to prevent alcoholism in a subject. Another outcome of prevention may be to administer the compounds or combinations of the present invention to prevent alcohol intake in a subject at risk of alcoholism or developing an alcohol-related disorder in a subject. Moreover, if treatment is commenced in a subject already consuming alcohol, such treatment may prevent the occurrence, progression or severity of alcohol-related disorders.

The terms “administration of” and or “administering” a compound should be understood to mean providing a compound of the invention or a prodrug of a compound of the invention to a subject in need of treatment. The administration of the compounds of the present invention in order to practice the present methods of therapy is carried out by administering a therapeutically effective amount of the compound to a subject in need of such treatment or prophylaxis. The need for a prophylactic administration according to the methods of the present invention is determined via the use of well known risk factors.

The term “therapeutically effective amount” as used herein means the amount of the active compound that will elicit the biological or medical response in a tissue, system, subject, mammal, or human that is being sought by the researcher, veterinarian, medical doctor or other clinician, which includes alleviation of the symptoms of the disorder being treated. The novel methods of treatment of this invention are for disorders known to those skilled in the art. The term “prophylactically effective amount” as used herein means the amount of the active compound that will elicit the biological or medical response in a tissue, system, subject, mammal, or human that is being sought by the researcher, veterinarian, medical doctor or other clinician, to prevent the onset of the disorder in subjects as risk for obesity or the disorder. The therapeutically or prophylactically effective amount, or dosage, of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgement.

### Administration and Dose Ranges

Any suitable route of administration may be employed for providing a subject or mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably the compound of Formula I, II, III, IV or V is administered orally or topically.

The effective dosage of active ingredient employed may vary depending on the particular compound employed, the mode of administration, the condition being treated and the severity of the condition being treated. Such dosage may be ascertained readily by a person skilled in the art.

When treating obesity, in conjunction with diabetes and/or hyperglycemia, or alone, generally satisfactory results are obtained when the compound of formula I, II, III, IV or V is administered at a daily dosage of from about 0.001 milligram to about 50 milligrams per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating diabetes mellitus and/or hyperglycemia, as well as other diseases or disorders for which the compound of formula I, II, III, IV or V is useful, generally satisfactory results are obtained when the compounds of the present invention are administered at a daily dosage of from about 0.001 milligram to about 50 milligram per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating dyslipidemia, bulimia nervosa, and gallstones satisfactory results are obtained when the compound of formula I, II, III, IV or V is administered at a daily dosage of from about 0.001 milligram to about 50 milligrams per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

In the case where an oral composition is employed, a suitable dosage range is, e.g. from about 0.01 mg to about 1500 mg of a compound of Formula I, II, III, IV or V per day, preferably from about 0.1 mg to about 600 mg per day, more preferably from about 0.1 mg to about 100 mg per day. For oral administration, the compositions are preferably provided in the form of tablets containing from 0.01 to 1,000 mg, preferably 0.01, 0.05, 0.1, 0.5, 1, 2.5, 5, 10, 15, 20, 25, 30, 40, 50, 100, 250, 500, 600, 750, 1000, 1250 or 1500 milligrams of the active ingredient for the symptomatic adjustment of the dosage to the patient to be treated.

For use where a composition for intranasal administration is employed, intranasal formulations for intranasal administration comprising 0.001-10% by weight solutions or suspensions of the compound of formula I, II, III, IV or V in an acceptable intranasal formulation may be used.

For use where a composition for intravenous administration is employed, a suitable dosage range is from about 0.001 mg to about 50 mg, preferably from 0.01 mg to about 50 mg, more preferably 0.1 mg to 10 mg, of a compound of formula I, II, III, IV or V per kg of body weight per day. This dosage regimen may be adjusted to provide the optimal therapeutic response. It may be necessary to use dosages outside these limits in some cases.

For the treatment of diseases of the eye, ophthalmic preparations for ocular administration comprising 0.001-1% by weight solutions or suspensions of the compound of formula I, II, III, IV or V in an acceptable ophthalmic formulation may be used.

The magnitude of prophylactic or therapeutic dosage of the compounds of the present invention will, of course, vary depending on the particular compound employed, the mode of administration, the condition being treated and the severity of the condition being treated. It will also vary according to the age, weight and response of the individual patient. Such dosage may be ascertained readily by a person skilled in the art.

A Compound of formula I, II, III, IV or V may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I.

Examples of other active ingredients that may be combined with a compound of Formula I for the treatment or prevention of obesity and/or diabetes, either administered separately or in the same pharmaceutical compositions, include, but are not limited to:

(a) insulin sensitizers including (i) PPARγ antagonists such as glitazones (e.g. ciglitazone; darglitazone; englitazone; isaglitazone (MCC-555); pioglitazone; rosiglitazone; troglitazone; tularik; BRL49653; CLX-0921; 5-BTZD), GW-0207, LG-100641, and LY-300512, and the like), and compounds disclosed in WO 97/10813, WO 97/27857, WO 97/28115, WO 97/28137, and WO 97/27847; (iii) biguanides such as metformin and phenformin;

(b) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulinotropin); and GLP-1 (7-36)-NH₂);

(c) sulfonylureas, such as acetohexamide; chlorpropamide; diabinese; glibenclamide; glipizide; glyburide; glimepiride; gliclazide; glipentide; gliquidone; glisolamide; tolazamide; and tolbutamide;

(d) α-glucosidase inhibitors, such as acarbose, adiposine; camiglibose; emiglitate; miglitol; voglibose; pradimicin-Q; salbostatin; CKD-711; MDL-25,637; MDL-73,945; and MOR 14, and the like;

(e) cholesterol lowering agents such as (i) HMG-CoA reductase inhibitors (atorvastatin, itavastatin, fluvastatin, lovastatin, pravastatin, rivastatin, rosuvastatin, simvastatin, and other statins), (ii) bile acid absorbers/sequestrants, such as cholestyramine, colestipol, dialkylaminoalkyl derivatives of a cross-linked dextran; Colestid®; LoCholest®, and the like, (ii) nicotinyl alcohol, nicotinic acid or a salt thereof, (iii) proliferator-activater receptor α agonists such as fenofibric acid derivatives (gemfibrozil, clofibrate, fenofibrate and benzafibrate), (iv) inhibitors of cholesterol absorption such as stanol esters, beta-sitosterol, sterol glycosides such as tiqueside; and azetidinones such as ezetimibe, and the like, and (acyl CoA:cholesterol acyltransferase (ACAT)) inhibitors such as avasimibe, and melinamide, (v) anti-oxidants, such as probucol, (vi) vitamin E, and (vii) thyromimetics;

(f) PPARα agonists such as beclofibrate, benzafibrate, ciprofibrate, clofibrate, etofibrate, fenofibrate, and gemfibrozil; and other fibric acid derivatives, such as Atromid®, Lopid® and Tricor®, and the like, and PPARα agonists as described in WO 97/36579 by Glaxo;

(g) PPARδ agonists, such as those disclosed in WO97/28149;

(h) PPAR α/δ agonists, such as muraglitazar, and the compounds disclosed in U.S. Pat. No. 6,414,002;

(i) smoking cessation agents, such as a nicotine agonist or a partial nicotine agonist such as varenicline, or a monoamine oxidase inhibitor (MAOI), or another active ingredient demonstrating efficacy in aiding cessation of tobacco consumption; for example, an antidepressant such as bupropion, doxepine, omortriptyline; or an anxiolytic such as buspirone or clonidine; and

(i) anti-obesity agents, such as (1) growth hormone secretagogues, growth hormone secretagogue receptor agonists/antagonists, such as NN703, hexarelin, MK-0677, SM-130686, CP-424,391, L-692,429, and L-163,255, and such as those disclosed in U.S. Pat. Nos. 5,536,716, and 6,358,951, U.S. Patent Application Nos. 2002/049196 and 2002/022637, and PCT Application Nos. WO 01/56592 and WO 02/32888; (2) protein tyrosine phosphatase-1B (PTP-1B) inhibitors; (3) cannabinoid receptor ligands, such as cannabinoid CB₁ receptor antagonists or inverse agonists, such as rimonabant (Sanofi Synthelabo), AMT-251, and SR-14778 and SR 141716A (Sanofi Synthelabo), SLV-319 (Solvay), BAY 65-2520 (Bayer), and those disclosed in U.S. Pat. Nos. 5,532,237, 4,973,587, 5,013,837, 5,081,122, 5,112,820, 5,292,736, 5,624,941, 6,028,084, PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, WO 01/64632, WO 01/64633, WO 01/64634, WO02/076949, WO 03/007887, WO 04/048317, and WO 05/000809; and EPO Application No. EP-658546, EP-656354, EP-576357; (4) anti-obesity serotonergic agents, such as fenfluramine, dexfenfluramine, phentermine, and sibutramine; (5) β3-adrenoreceptor agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, SR 59119A, and such as those disclosed in U.S. Pat. Nos. 5,705,515, and U.S. Pat. No. 5,451,677 and PCT Patent Publications WO94/18161, WO95/29159, WO97/46556, WO98/04526 and WO98/32753, WO 01/74782, and WO 02/32897; (6) pancreatic lipase inhibitors, such as orlistat (Xenical®), Triton WR1339, RHC80267, lipstatin, tetrahydrolipstatin, teasaponin, diethylumbelliferyl phosphate, and those disclosed in PCT Application No. WO 01/77094; (7) neuropeptide Y1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; (8) neuropeptide Y5 antagonists, such as GW-569180A, GW-594884A, GW-587081×, GW-548118×, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, hereby incorporated by reference in their entirety; European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; WO 02/094825; WO 03/014083; WO 03/10191; WO 03/092889; WO 04/002986; and WO 04/031175; (9) melanin-concentrating hormone (MCH) receptor antagonists, such as those disclosed in WO 01/21577 and WO 01/21169; (10) melanin-concentrating hormone 1 receptor (MCH1R) antagonists, such as T-226296 (Takeda), and those disclosed in PCT Patent Application Nos. WO 01/82925, WO 01/87834, WO 02/051809, WO 02/06245, WO 02/076929, WO 02/076947, WO 02/04433, WO 02/51809, WO 02/083134, WO 02/094799, WO 03/004027, and Japanese Patent Application Nos. JP 13226269, and JP 2004-139909; (11) melanin-concentrating hormone 2 receptor (MCH2R) agonist/antagonists; (12) orexin-1 receptor antagonists, such as SB-334867-A, and those disclosed in PCT Patent Application Nos. WO 01/96302, WO 01/68609, WO 02/51232, and WO 02/51838; (13) serotonin reuptake inhibitors such as fluoxetine, paroxetine, and sertraline, and those disclosed in U.S. Pat. No. 6,365,633, and PCT Patent Application Nos. WO 01/27060 and WO 01/162341; (14) melanocortin agonists, such as Melanotan II or those described in WO 99/64002 and WO 00/74679; (15) other Mc4r (melanocortin 4 receptor) agonists, such as CHIR86036 (Chiron), ME-10142, and ME-10145 (Melacure), CHIR86036 (Chiron); PT-141, and PT-14 (Palatin), and those disclosed in: U.S. Pat. Nos. 6,410,548; 6,294,534; 6,350,760; 6,458,790; 6,472,398; 6,376,509; and 6,818,658; US Patent Publication No. US2002/0137664; US2003/0236262; US2004/009751; US2004/0092501; and PCT Application Nos. WO 99/64002; WO 00/74679; WO 01/70708; WO 01/70337; WO 01/74844; WO 01/91752; WO 01/991752; WO 02/15909; WO 02/059095; WO 02/059107; WO 02/059108; WO 02/059117; WO 02/067869; WO 02/068387; WO 02/068388; WO 02/067869; WO 02/11715; WO 02/12166; WO 02/12178; WO 03/007949; WO 03/009847; WO 04/024720; WO 04/078716; WO 04/078717; WO 04/087159; WO 04/089307; and WO 05/009950; (16) 5HT-2 agonists; (17) 5HT2C (serotonin receptor 2C) agonists, such as BVT933, DPCA37215, WAY161503, R-1065, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; (18) galanin antagonists; (19) CCK agonists; (20) CCK-A (cholecystokinin-A) agonists, such as AR-R 15849, GI 181771, JMV-180, A-71378, A-71623 and SR146131, and those described in U.S. Pat. No. 5,739,106; (21) GLP-1 agonists; (22) corticotropin-releasing hormone agonists; (23) histamine receptor-3 (H3) modulators; (24) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, GT2394 (Gliatech), and those described and disclosed in PCT Application No. WO 02/15905, and O-[3-(1H-imidazol-4-yl)propanol]-carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)); (25) β-hydroxy steroid dehydrogenase-1 inhibitors (β-HSD-1); 26) PDE (phosphodiesterase) inhibitors, such as theophylline, pentoxifylline, zaprinast, sildenafil, amrinone, milrinone, cilostamide, rolipram, and cilomilast; (27) phosphodiesterase-3B (PDE3B) inhibitors; (28) NE (norepinephrine) transport inhibitors, such as GW 320659, despiramine, talsupram, and nomifensine; (29) ghrelin receptor antagonists, such as those disclosed in PCT Application Nos. WO 01/87335, and WO 02/08250; (30) leptin, including recombinant human leptin (PEG-OB, Hoffman La Roche) and recombinant methionyl human leptin (Amgen); (31) leptin derivatives, such as those disclosed in U.S. Pat. Nos. 5,552,524, 5,552,523, 5,552,522, 5,521,283, and PCT International Publication Nos. WO 96/23513, WO 96/23514, WO 96/23515, WO 96/23516, WO 96/23517, WO 96/23518, WO 96/23519, and WO 96/23520; (32) BRS3 (bombesin receptor subtype 3) agonists such as [D-Phe6,beta-Ala11,Phe13,Nle14]Bn(6-14) and [D-Phe6,Phe13]Bn(6-13)propylamide, and those compounds disclosed in Pept. Sci. 2002 August; 8(8): 461-75); (33) CNTF (Ciliary neurotrophic factors), such as GI-181771 (Glaxo-SmithKline), SR146131 (Sanofi Synthelabo), butabindide, PD170,292, and PD 149164 (Pfizer); (34) CNTF derivatives, such as axokine (Regeneron), and those disclosed in PCT Application Nos. WO 94/09134, WO 98/22128, and WO 99/43813; (35) monoamine reuptake inhibitors, such as sibutramine, and those disclosed in U.S. Pat. Nos. 4,746,680, 4,806,570, and 5,436,272, U.S. Patent Publication No. 2002/0006964 and PCT Application Nos. WO 01/27068, and WO 01/62341; (36) UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in PCT Patent Application No. WO 99/00123; (37) thyroid hormone β agonists, such as KB-2611 (KaroBioBMS), and those disclosed in PCT Application No. WO 02/15845, and Japanese Patent Application No. JP 2000256190; (38) FAS (fatty acid synthase) inhibitors, such as Cerulenin and C75; (39) DGAT1 (diacylglycerol acyltransferase 1) inhibitors; (40) DGAT2 (diacylglycerol acyltransferase 2) inhibitors; (41) ACC2 (acetyl-CoA carboxylase-2) inhibitors; (42) glucocorticoid antagonists; (43) acyl-estrogens, such as oleoyl-estrone, disclosed in del Mar-Grasa, M. et al., Obesity Research, 9:202-9 (2001); (44) dipeptidyl peptidase IV (DP-IV) inhibitors, such as isoleucine thiazolidide, valine pyrrolidide, NVP-DPP728, LAF237, P93/01, TSL 225, TMC-2A/2B/2C, FE 999011, P9310/K364, VIP 0177, SDZ 274-444 and sitagliptin; and the compounds disclosed in U.S. Pat. No. 6,699,871, which is incorporated herein by reference; and International Patent Application Nos. WO 03/004498; WO 03/004496; EP 1 258 476; WO 02/083128; WO 02/062764; WO 03/000250; WO 03/002530; WO 03/002531; WO 03/002553; WO 03/002593; WO 03/000180; and WO 03/000181; (46) dicarboxylate transporter inhibitors; (47) glucose transporter inhibitors; (48) phosphate transporter inhibitors; (49) Metformin (Glucophage®); and (50) Topiramate (Topimax®); and (50) peptide YY, PYY 3-36, peptide YY analogs, derivatives, and fragments such as BIM-43073D, BIM-43004C (Olitvak, D. A. et al., Dig. Dis. Sci. 44(3):643-48 (1999)), and those disclosed in U.S. Pat. No. 5,026,685, U.S. Pat. No. 5,604,203, U.S. Pat. No. 5,574,010, U.S. Pat. No. 5,696,093, U.S. Pat. No. 5,936,092, U.S. Pat. No. 6,046,162, U.S. Pat. No. 6,046,167, U.S. Pat. No. 6,093,692, U.S. Pat. No. 6,225,445, U.S. Pat. No. 5,604,203, U.S. Pat. No. 4,002,531, U.S. Pat. No. 4, 179,337, U.S. Pat. No. 5,122,614, U.S. Pat. No. 5,349,052, U.S. Pat. No. 5,552,520, U.S. Pat. No. 6,127,355, WO 95/06058, WO 98/32466, WO 03/026591, WO 03/057235, WO 03/027637, and WO 2004/066966, which are incorporated herein by reference; (51) Neuropeptide Y2 (NPY2) receptor agonists such NPY3-36, N acetyl [Leu(28,31)] NPY 24-36, TASP-V, and cyclo-(28/32)-Ac-[Lys28-Glu32]-(25-36)-pNPY; (52) Neuropeptide Y4 (NPY4) agonists such as pancreatic peptide (PP) as described in Batterham et al., J. Clin. Endocrinol. Metab. 88:3989-3992 (2003), and other Y4 agonists such as 1229U91; (54) cyclo-oxygenase-2 inhibitors such as etoricoxib, celecoxib, valdecoxib, parecoxib, lumiracoxib, BMS347070, tiracoxib or JTE522, ABT963, CS502 and GW406381, and pharmaceutically acceptable salts thereof; (55) Neuropeptide Y1 (NPY1) antagonists such as BIBP3226, J-115814, BIBO 3304, LY-357897, CP-671906, GI-264879A and those disclosed in U.S. Pat. No. 6,001,836; and PCT Application Nos. WO 96/14307, WO 01/23387, WO 99/51600, WO 01/85690, WO 01/85098, WO 01/85173, and WO 01/89528; (56) Opioid antagonists such as nalmefene (Revex®), 3-methoxynaltrexone, naloxone, naltrexone, and those disclosed in: PCT Application No. WO 00/21509; (57)11β HSD-1 (11-beta hydroxy steroid dehydrogenase type 1) inhibitor such as BVT 3498, BVT 2733, and those disclosed in WO 01/90091, WO 01/90090, WO 01/90092, and U.S. Pat. No. 6,730,690 and US Publication No. US 2004-0133011, which are incorporated by reference herein in their entirety; and (58) aminorex; (59) amphechloral; (60) amphetamine; (61) benzphetamine; (62) chlorphentermine; (63) clobenzorex; (64) cloforex; (65) clominorex; (66) clortermine; (67) cyclexedrine; (68) dextroamphetamine; (69) diphemethoxidine, (70) N-ethylamphetamine; (71) fenbutrazate; (72) fenisorex; (73) fenproporex; (74) fludorex; (75) fluminorex; (76) furfurylmethylamphetamine; (77) levamfetamine; (78) levophacetoperane; (79) mefenorex; (80) metamfepramone; (81) methamphetamine; (82) norpseudoephedrine; (83) pentorex; (84) phendimetrazine; (85) phenmetrazine; (86) picilorex; (87) phytopharm 57; (88) zonisamide, (89) neuromedin U and analogs or derivatives thereof, (90) oxyntomodulin and analogs or derivatives thereof, and (91) Neurokinin-1 receptor antagonists (NK-1 antagonists) such as the compounds disclosed in: U.S. Pat. Nos. 5,162,339, 5,232,929, 5,242,930, 5,373,003, 5,387,595, 5,459,270, 5,494,926, 5,496,833, and 5,637,699.

Specific compounds of use in combination with a compound of the present invention include: simvastatin, mevastatin, ezetimibe, atorvastatin, sitagliptin, metformin, sibutramine, orlistat, Qnexa, topiramate, naltrexone, bupriopion, phentermine, and losartan, losartan with hydrochlorothiazide. Specific CB1 antagonists/inverse agonists of use in combination with a compound of the present invention include: those described in WO03/077847, including: N-[3-(4-chlorophenyl)-2(S)-phenyl-1(S)-methylpropyl]-2-(4-trifluoromethyl-2-pyrimidyloxy)-2-methylpropanamide, N-[3-(4-chlorophenyl)-2-(3-cyanophenyl)-1-methylpropyl]-2-(5-trifluoromethyl-2-pyridyloxy)-2-methylpropanamide, N-[3-(4-chlorophenyl)-2-(5-chloro-3-pyridyl)-1-methylpropyl]-2-(5-trifluoromethyl-2-pyridyloxy)-2-methylpropanamide, and pharmaceutically acceptable salts thereof; as well as those in WO05/000809, which includes the following: 3-{1-[bis(4-chlorophenyl)methyl]azetidin-3-ylidene}-3-(3,5-difluorophenyl)-2,2-dimethylpropanenitrile, 1-{1-[1-(4-chlorophenyl)pentyl]azetidin-3-yl}-1-(3,5-difluorophenyl)-2-methylpropan-2-ol. 3-((S)-(4-chlorophenyl) {3-[(1S)-1-(3,5-difluorophenyl)-2-hydroxy-2-methylpropyl] azetidin-1-yl}methyl)benzonitrile, 3-((S)-(4-chlorophenyl) {3-[(1S)-1-(3,5-difluorophenyl)-2-fluoro-2-methylpropyl]azetidin-1-yl}methyl)benzonitrile, 3-((4-chlorophenyl) {3-[1-(3,5-difluorophenyl)-2,2-dimethylpropyl]azetidin-1-yl}methyl)benzonitrile, 3-((1S)-1-{1-[(S)-(3-cyanophenyl)(4-cyanophenyl)methyl]azetidin-3-yl}-2-fluoro-2-methylpropyl)-5-fluorobenzonitrile, 3-[(S)-(4-chlorophenyl)(3-{(1S)-2-fluoro-1-[3-fluoro-5-(4H-1,2,4-triazol-4-yl)phenyl]-2-methylpropyl}azetidin-1-yl)methyl]benzonitrile, and 5-((4-chlorophenyl) {3-[(1S)-1-(3,5-difluorophenyl)-2-fluoro-2-methylpropyl]azetidin-1-yl}methyl)thiophene-3-carbonitrile, and pharmaceutically acceptable salts thereof; as well as: 3-[(S)-(4-chlorophenyl)(3-{(1s)-2-fluoro-1-[3-fluoro-5-(5-oxo-4,5-dihydro-1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(3-{(1S)-1-[3-(5-amino-1,3,4-oxadiazol-2-yl)-5-fluorophenyl]-2-fluoro-2-methylpropyl}azetidin-1-yl)(4-chlorophenyl)methyl]benzonitrile, 3-[(S)-(4-cyanophenyl)(3-{(1S)-2-fluoro-1-[3-fluoro-5-(5-oxo-4,5-dihydro-1,3,4-oxadiazol-2-yl)phenyl]-2-methylpropyl}azetidin-1-yl)methyl]benzonitrile, 3-[(S)-(3-{(1S)-1-[3-(5-amino-1,3,4-oxadiazol-2-yl)-5-fluorophenyl]-2-fluoro-2-methylpropyl}azetidin-1-yl)(4-cyanophenyl)methyl]benzonitrile, 3-[(S)-(4-cyanophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,2,4-oxadiazol-3-yl)phenyl]-2-methylpropyl}azetidin-1-yl)methyl]benzonitrile, 3-[(1S)-1-(1-{(S)-(4-cyanophenyl)[3-(1,2,4-oxadiazol-3-yl)phenyl]-methyl}azetidin-3-yl)-2-fluoro-2-methylpropyl]-5-fluorobenzonitrile, 5-(3-{1-[1-(diphenylmethyl)azetidin-3-yl]-2-fluoro-2-methylpropyl}-5-fluorophenyl)-1H-tetrazole, 5-(3-{1-[1-(diphenylmethyl)azetidin-3-yl]-2-fluoro-2-methylpropyl}-5-fluorophenyl)-1-methyl-1H-tetrazole, 5-(3-{1-[1-(diphenylmethyl)azetidin-3-yl]-2-fluoro-2-methylpropyl}-5-fluorophenyl)-2-methyl-2H-tetrazole, 3-[(4-chlorophenyl)(3-{2-fluoro-1-[3-fluoro-5-(2-methyl-2H-tetrazol-5-yl)phenyl]-2-methylpropyl}azetidin-1-yl)methyl]benzonitrile, 3-[(4-chlorophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(2-methyl-2H-tetrazol-5-yl)phenyl]-2-methylpropyl}azetidin-1-yl)methyl]benzonitrile, 5-{3-[(S)-{3-[(1S)-1-(3-bromo-5-fluorophenyl)-2-fluoro-2-methylpropyl]azetidin-1-yl}(4-chlorophenyl)methyl]phenyl}-1,3,4-oxadiazol-2(3H)-one, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-cyanophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-cyanophenyl)[3-(1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-chlorophenyl)[3-(1,3,4-oxadiazol-2-yl)phenyl]methyl}azetidin-3-yl)-2-fluoro-2-methylpropyl]-5-fluorobenzonitrile, 3-((1S)-1-{1-[(S)-[3-(5-amino-1,3,4-oxadiazol-2-yl)phenyl](4-chlorophenyl)methyl]azetidin-3-yl}-2-fluoro-2-methylpropyl)-5-fluorobenzonitrile, 3-((1S)-1-{1-[(S)-[3-(5-amino-1,3,4-oxadiazol-2-yl)phenyl](4-cyanophenyl)methyl]azetidin-3-yl}-2-fluoro-2-methylpropyl)-5-fluorobenzonitrile, 3-[(1S)-1-(1-{(S)-(4-cyanophenyl)[3-(1,2,4-oxadiazol-3-yl)phenyl]methyl}azetidin-3-yl)-2-fluoro-2-methylpropyl]-5-fluorobenzonitrile, 3-[(1S)-1-(1-{(S)-(4-chlorophenyl)[3-(1,2,4-oxadiazol-3-yl)phenyl]methyl}azetidin-3-yl)-2-fluoro-2-methylpropyl]-5-fluorobenzonitrile, 5-[3-((S)-(4-chlorophenyl) {3-[(1S)-1-(3,5-difluorophenyl)-2-fluoro-2-methylpropyl]azetidin-1-yl}methyl)phenyl]-1,3,4-oxadiazol-2(3H)-one, 5-[3-((S)-(4-chlorophenyl) {3-[(1S)-1-(3,5-difluorophenyl)-2-fluoro-2-methylpropyl]azetidin-1-yl}methyl)phenyl]-1,3,4-oxadiazol-2(3H)-one, 4-{(S)-{3-[(1S)-1-(3,5-difluorophenyl)-2-fluoro-2-methylpropyl]azetidin-1-yl}[3-(5-oxo-4,5-dihydro-1,3,4-oxadiazol-2-yl)phenyl]methyl}-benzonitrile, and pharmaceutically acceptable salts thereof.

Specific NPY5 antagonists of use in combination with a compound of the present invention include: 3-oxo-N-(5-phenyl-2-pyrazinyl)-spiro[isobenzofuran-1(3H), 4′-piperidine]-1′-carboxamide, 3-oxo-N-(7-trifluoromethylpyrido[3,2-b]pyridin-2-yl)spiro-[isobenzofuran-1(3H), 4′-piperidine]-1′-carboxamide, N-[5-(3-fluorophenyl)-2-pyrimidinyl]-3-oxospiro-[isobenzofuran-1(3H), 4′-piperidine]-1′-carboxamide, trans-3′-oxo-N-(5-phenyl-2-pyrimidinyl)spiro[cyclohexane-1,1′(3′H)-isobenzofuran]-4-carboxamide, trans-3′-oxo-N-[1-(3-quinolyl)-4-imidazolyl]spiro[cyclohexane-1,1′(3′H)-isobenzofuran]-4-carboxamide, trans-3-oxo-N-(5-phenyl-2-pyrazinyl)spiro[4-azaiso-benzofuran-1(3H), 1′-cyclohexane]-4′-carboxamide, trans-N-[5-(3-fluorophenyl)-2-pyrimidinyl]-3-oxospiro[5-azaisobenzofuran-1(3H), 1′-cyclohexane]-4′-carboxamide, trans-N-[5-(2-fluorophenyl)-2-pyrimidinyl]-3-oxospiro[5-azaisobenzofuran-1(3H), 1′-cyclohexane]-4′-carboxamide, trans-N-[1-(3,5-difluorophenyl)-4-imidazolyl]-3-oxospiro[7-azaisobenzofuran-1(3H), 1′-cyclohexane]-4′-carboxamide, trans-3-oxo-N-(1-phenyl-4-pyrazolyl)spiro[4-azaisobenzofuran-1(3H), 1′-cyclohexane]-4′-carboxamide, trans-N-[1-(2-fluorophenyl)-3-pyrazolyl]-3-oxospiro[6-azaisobenzofuran-1(3H), 1′-cyclohexane]-4′-carboxamide, trans-3-oxo-N-(1-phenyl-3-pyrazolyl)spiro[6-azaisobenzofuran-1(3H), 1′-cyclohexane]-4′-carboxamide, trans-3-oxo-N-(2-phenyl-1,2,3-triazol-4-yl)spiro[6-azaisobenzofuran-1(3H), 1′-cyclohexane]-4′-carboxamide, and pharmaceutically acceptable salts and esters thereof.

Specific ACC-1/2 inhibitors of use in combination with a compound of the present invention include: 1′-[(4,8-dimethoxyquinolin-2-yl)carbonyl]-6-(1H-tetrazol-5-yl)spiro[chroman-2,4′-piperidin]-4-one; (5-{1′-[(4,8-dimethoxyquinolin-2-yl)carbonyl]-4-oxospiro[chroman-2,4′-piperidin]-6-yl}-2H-tetrazol-2-yl)methyl pivalate; 5-{1′-[(8-cyclopropyl-4-methoxyquinolin-2-yl)carbonyl]-4-oxospiro[chroman-2,4′-piperidin]-6-yl}nicotinic acid; 1′-(8-methoxy-4-morpholin-4-yl-2-naphthoyl)-6-(1H-tetrazol-5-yl)spiro[chroman-2,4′-piperidin]-4-one; and 1′-[(4-ethoxy-8-ethylquinolin-2-yl)carbonyl]-6-(1H-tetrazol-5-yl)spiro[chroman-2,4′-piperidin]-4-one; and pharmaceutically acceptable salts and esters thereof. Specific MCH1R antagonist compounds of use in combination with a compound of the present invention include: 1-{4-[(1-ethylazetidin-3-yl)oxy]phenyl}-4-[(4-fluorobenzyl)oxy]pyridin-2(1H)-one, 4-[(4-fluorobenzyl)oxy]-1-{4-[(1-isopropylazetidin-3-yl)oxy]phenyl}pyridin-2(1H)-one, 1-[4-(azetidin-3-yloxy)phenyl]-4-[(5-chloropyridin-2-yl)methoxy]pyridin-2(1H)-one, 4-[(5-chloropyridin-2-yl)methoxy]-1-{4-[(1-ethylazetidin-3-yl)oxy]phenyl}pyridin-2(1H)-one, 4-[(5-chloropyridin-2-yl)methoxy]1-{4-[(1-propylazetidin-3-yl)oxy]phenyl}pyridin-2(1H)-one, and 4-[(5-chloropyridin-2-yl)methoxy]-1-(4-{[(2S)-1-ethylazetidin-2-yl]methoxy}phenyl)pyridin-2(1H)-one, or a pharmaceutically acceptable salt thereof. Specific DP-IV inhibitors of use in combination with a compound of the present invention are selected from 7-[(3R)-3-amino-4-(2,4,5-trifluorophenyl)butanoyl]-3-(trifluoromethyl)-5,6,7,8-tetrahydro-1,2,4-triazolo[4,3-a]pyrazine. In particular, the compound of formula I is favorably combined with 7-[(3R)-3-amino-4-(2,4,5-trifluorophenyl)butanoyl]-3-(trifluoromethyl)-5,6,7,8-tetrahydro-1,2,4-triazolo[4,3-a]pyrazine, and pharmaceutically acceptable salts thereof.

Specific H3 (histamine H3) antagonists/inverse agonists of use in combination with a compound of the present invention include: those described in WO05/077905, including: 3-{4-[(1-cyclobutyl-4-piperidinyl)oxy]phenyl}-2-ethylpyrido[2,3-d]-pyrimidin-4(3H)-one, 3-{4-[(1-cyclobutyl-4-piperidinyl)oxy]phenyl}-2-methylpyrido[4,3-d]pyrimidin-4(3H)-one, 2-ethyl-3-(4-{3-[(3S)-3-methylpiperidin-1-yl]propoxy}phenyl)pyrido[2,3-d]pyrimidin-4(3H)-one 2-methyl-3-(4-{3-[(3S)-3-methylpiperidin-1-yl]propoxy}phenyl)pyrido[4,3-d]pyrimidin-4(3H)-one, 3-{4-[(1-cyclobutyl-4-piperidinyl)oxy]phenyl}-2,5-dimethyl-4(3H)-quinazolinone, 3-{4-[(1-cyclobutyl-4-piperidinyl)oxy]phenyl}-2-methyl-5-trifluoromethyl-4(3H)-quinazolinone, 3-{4-[(1-cyclobutyl-4-piperidinyl)oxy]phenyl}-5-methoxy-2-methyl-4(3H)-quinazolinone, 3-{4-[(1-cyclobutylpiperidin-4-yl)oxy]phenyl}-5-fluoro-2-methyl-4(3H)-quinazolinone, 3-{4-[(1-cyclobutylpiperidin-4-yl)oxy]phenyl}-7-fluoro-2-methyl-4(3H)-quinazolinone, 3-{4-[(1-cyclobutylpiperidin-4-yl)oxy]phenyl}-6-methoxy-2-methyl-4(3H)-quinazolinone, 3-{4-[(1-cyclobutylpiperidin-4-yl)oxy]phenyl}-6-fluoro-2-methyl-4(3H)-quinazolinone, 3-{4-[(1-cyclobutylpiperidin-4-yl)oxy]phenyl}-8-fluoro-2-methyl-4(3H)-quinazolinone, 3-{4-[(1-cyclopentyl-4-piperidinyl)oxy]phenyl}-2-methylpyrido[4,3-d]pyrimidin-4(3H)-one, 3-{4-[(1-cyclobutylpiperidin-4-yl)oxy]phenyl}-6-fluoro-2-methylpyrido[3,4-d]pyrimidin-4(3H)-one, 3-{4-[(1-cyclobutyl-4-piperidinyl)oxy]phenyl}-2-ethylpyrido[4,3-d]pyrimidin-4(3H)-one, 6-methoxy-2-methyl-3-{4-[3-(1-piperidinyl)propoxy]phenyl}pyrido[3,4-d]pyrimidin-4(3H)-one, 6-methoxy-2-methyl-3-{4-[3-(1-pyrrolidinyl)propoxy]phenyl}pyrido[3,4-d]pyrimidin-4(3H)-one, 2,5-dimethyl-3-{4-[3-(1-pyrrolidinyl)propoxy]phenyl}-4(3H)-quinazolinone, 2-methyl-3-{4-[3-(1-pyrrolidinyl)propoxy]phenyl}-5-trifluoromethyl-4(3H)-quinazolinone, 5-fluoro-2-methyl-3-{4-[3-(1-piperidinyl)propoxy]phenyl}-4(3H)-quinazolinone, 6-methoxy-2-methyl-3-{4-[3-(1-piperidinyl)propoxy]phenyl}-4(3H)-quinazolinone, 5-methoxy-2-methyl-3-(4-{3-[(3S)-3-methylpiperidin-1-yl]propoxy}phenyl)-4(3H)-quinazolinone, 7-methoxy-2-methyl-3-(4-{3-[(3S)-3-methylpiperidin-1-yl]propoxy}phenyl)-4(3H)-quinazolinone, 2-methyl-3-(4-{3-[(3S)-3-methylpiperidin-1-yl]propoxy}phenyl)pyrido[2,3-d]pyrimidin-4(3H)-one, 5-fluoro-2-methyl-3-(4-{3-[(2R)-2-methylpyrrolidin-1-yl]propoxy}phenyl)-4(3H)-quinazolinone, 2-methyl-3-(4-{3-[(2R)-2-methylpyrrolidin-1-yl]propoxy}phenyl)pyrido[4,3-d]pyrimidin-4(3H)-one, 6-methoxy-2-methyl-3-(4-{3-[(2R)-2-methylpyrrolidin-1-yl]propoxy}phenyl)-4(3H)-quinazolinone, 6-methoxy-2-methyl-3-(4-{3-[(2S)-2-methylpyrrolidin-1-yl]propoxy}phenyl)-4(3H)-quinazolinone, and pharmaceutically acceptable salts thereof.

Specific CCK1R agonists of use in combination with a compound of the present invention include: 3-(4-{[1-(3-ethoxyphenyl)-2-(4-methylphenyl)-1H-imidazol-4-yl]carbonyl}-1-piperazinyl)-1-naphthoic acid; 3-(4-{[1-(3-ethoxyphenyl)-2-(2-fluoro-4-methylphenyl)-1H-imidazol-4-yl]carbonyl}-1-piperazinyl)-1-naphthoic acid; 3-(4-{[1-(3-ethoxyphenyl)-2-(4-fluorophenyl)-1H-imidazol-4-yl]carbonyl}-1-piperazinyl)-1-naphthoic acid; 3-(4-{[1-(3-ethoxyphenyl)-2-(2,4-difluorophenyl)-1H-imidazol-4-yl]carbonyl}-1-piperazinyl)-1-naphthoic acid; and 3-(4-{[1-(2,3-dihydro-1,4-benzodioxin-6-yl)-2-(4-fluorophenyl)-1H-imidazol-4-yl]carbonyl}-1-piperazinyl)-1-naphthoic acid; and pharmaceutically acceptable salts thereof. Specific MC4R agonists of use in combination with a compound of the present invention include: 1) (5S)-1′-{[(3R,4R)-1-tert-butyl-3-(2,3,4-trifluorophenyl)piperidin-4-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 2) (5R)-1′-{[(3R,4R)-1-tert-butyl-3-(2,3,4-trifluorophenyl)-piperidin-4-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 3) 2-(11′-{[(3S,4R)-1-tert-butyl-4-(2,4-difluorophenyl)pyrrolidin-3-yl]carbonyl}-3-chloro-2-methyl-5H-spiro[furo[3,4-b]pyridine-7,4′-piperidin]-5-yl)-2-methylpropanenitrile; 4) 1′-{[(3S,4R)-1-tert-butyl-4-(2,4-difluorophenyl)pyrrolidin-3-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 5) N-[(3R,4R)-3-({3-chloro-2-methyl-5-[1-methyl-1-(1-methyl-1H-1,2,4-triazol-5-yl)ethyl]-1′H,5H-spiro[furo-[3,4-b]pyridine-7,4′-piperidin]-1′-yl}carbonyl)-4-(2,4-difluorophenyl)-cyclopentyl]-N-methyltetrahydro-2H-pyran-4-amine; 6) 2-[3-chloro-1′-({(1R,2R)-2-(2,4-difluorophenyl)-4-[methyl(tetrahydro-2H-pyran-4-yl)amino]-cyclopentyl}-carbonyl)-2-methyl-5H-spiro[furo[3,4-b]pyridine-7,4′-piperidin]-5-yl]-2-methyl-propane-nitrile; and pharmaceutically acceptable salts thereof.

Examples of other anti-obesity agents that can be employed in combination with a compound of Formula I are disclosed in “Patent focus on new anti-obesity agents,” Exp. Opin. Ther. Patents, 10: 819-831 (2000); “Novel anti-obesity drugs,” Exp. Opin. Invest. Drugs, 9: 1317-1326 (2000); and “Recent advances in feeding suppressing agents: potential therapeutic strategy for the treatment of obesity, Exp. Opin. Ther. Patents, 11: 1677-1692 (2001). The role of neuropeptide Y in obesity is discussed in Exp. Opin. Invest. Drugs, 9: 1327-1346 (2000). Cannabinoid receptor ligands are discussed in Exp. Opin. Invest. Drugs, 9: 1553-1571 (2000).

Examples of other active ingredients that may be combined with a compound of Formula I for the treatment or prevention of male or female sexual dysfunction, in particular, male erectile dysfunction, either administered separately or in the same pharmaceutical compositions, include, but are not limited to (a) type V cyclic-GMP-specific phosphodiesterase (PDE-V) inhibitors, including sildenafil and (6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351); (b) alpha-adrenergic receptor antagonists, including phentolamine and yohimbine or pharmaceutically acceptable salts thereof; (c) dopamine receptor agonists, such as apomorphine or pharmaceutically acceptable salts thereof; and (d) nitric oxide (NO) donors.

The instant invention also includes administration of a single pharmaceutical dosage formulation which contains both the MC-4R agonist in combination with a second active ingredient, as well as administration of each active agent in its own separate pharmaceutical dosage formulation. Where separate dosage formulations are used, the individual components of the composition can be administered at essentially the same time, i.e., concurrently, or at separately staggered times, i.e. sequentially prior to or subsequent to the administration of the other component of the composition. The instant invention is therefore to be understood to include all such regimes of simultaneous or alternating treatment, and the terms “administration” and “administering” are to be interpreted accordingly. Administration in these various ways are suitable for the present compositions as long as the beneficial pharmaceutical effect of the combination of the MC-4R agonist and the second active ingredient is realized by the patient at substantially the same time. Such beneficial effect is preferably achieved when the target blood level concentrations of each active ingredient are maintained at substantially the same time. It is preferred that the combination of the MC-4R agonist and the second active ingredient be co-administered concurrently on a once-a-day dosing schedule; however, varying dosing schedules, such as the MC-4R agonist once a day and the second active ingredient once, twice or more times per day or the MC-4R agonist three times a day and the second active ingredient once, twice or more times per day, is also encompassed herein. A single oral dosage formulation comprised of both a MC-4R agonist and a second active ingredient is preferred. A single dosage formulation will provide convenience for the patient, which is an important consideration especially for patients with diabetes or obese patients who may be in need of multiple medications.

The compounds in the combinations of the present invention may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of the melanocortin-4 receptor agonist, or a pharmaceutically acceptable salt or ester thereof, and a pharmaceutically acceptable carrier or diluent in a first unit dosage form, and a second unit dosage form comprising a prophylactically or therapeutically effective amount of the second active ingredient or drug, or a pharmaceutically acceptable salt or ester thereof, and a pharmaceutically acceptable carrier or diluent in a second unit dosage form. In one embodiment, the kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

Another aspect of the present invention provides pharmaceutical compositions which comprise a compound of Formula I, as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the typical oral dosage unit form, in which case solid pharmaceutical carriers are typically employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The compounds of structural formula I of the present invention can be prepared according to the procedures of the following Schemes and Examples, using appropriate materials and are further exemplified by the following specific examples. Moreover, by utilizing the procedures described in detail in PCT International Application Publication WO 02/068387, and WO 02/068388 in conjunction with the disclosure contained herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described previously hereinabove. The free amine bases corresponding to the isolated salts can be generated by neutralization with a suitable base, such as aqueous sodium hydrogencarbonate, sodium carbonate, sodium hydroxide, and potassium hydroxide, and extraction of the liberated amine free base into an organic solvent followed by evaporation. The amine free base isolated in this manner can be further converted into another pharmaceutically acceptable salt by dissolution in an organic solvent followed by addition of the appropriate acid and subsequent evaporation, precipitation, or crystallization. All temperatures are degrees Celsius unless otherwise noted. Mass spectra (MS) were measured by electron-spray ion-mass spectroscopy.

The phrase “standard peptide coupling reaction conditions” means coupling a carboxylic acid with an amine using an acid activating agent such as EDC, DCC, and BOP in an inert solvent such as dichloromethane in the presence of a catalyst such as HOBT. The use of protecting groups for the amine and carboxylic acid functionalities to facilitate the desired reaction and minimize undesired reactions is well documented. Conditions required to remove protecting groups are found in standard textbooks such as Greene, T, and Wuts, P. G. M., Protective Groups in Organic Synthesis, John Wiley & Sons, Inc., New York, N.Y., 1991. CBZ and BOC are commonly used protecting groups in organic synthesis, and their removal conditions are known to those skilled in the art. For example, CBZ may be removed by catalytic hydrogenation in the presence of a noble metal or its oxide such as palladium on activated carbon in a protic solvent such as methanol or ethanol. In cases where catalytic hydrogenation is contraindicated due to the presence of other potentially reactive functionalities, removal of CBZ groups can also be achieved by treatment with a solution of hydrogen bromide in acetic acid or by treatment with a mixture of TFA and dimethylsulfide. Removal of BOC protecting groups is carried out with a strong acid, such as trifluoroacetic acid, hydrochloric acid, or hydrogen chloride gas, in a solvent such as methylene chloride, methanol, or ethyl acetate.

Abbreviations Used in the Description of the Preparation of the Compounds of the Present Invention: AcOH is acetic acid; aq or Aq is aqueous, AcCN is acetonitrile, BOC or Boc is t-butyloxycarbonyl, Boc₂ is Boc anhydride; BOP is benzotriazol-1-yloxytris(dimethylamino)-phosphonium hexafluorophosphate, Bn is benzyl, Bu is butyl, t-Bu is tert-butyl, t-BuOH is tert-butanol, calc. or calc'd is Calculated, celite is Celite™ diatomaceous earth, CBZ (Cbz) is benzyloxycarbonyl, c-hex is cyclohexyl, c-pen is cyclopentyl, c-pro is cyclopropyl, conc is concentrated, DCM is dichloromethane, DEAD is diethyl azodicarboxylate, DIPEA or DIEA is diisopropyl-ethylamine, DMA is dimethyl acetamide, DMAP is 4-dimethylaminopyridine, DMF is N,N-dimethyl-formamide, dppf is 1,1′-Bis(diphenylphosphino) ferrocene, DMSO is dimethyl sulfoxide, EDC is 1-(3-dimethylaminopropyl)-3-ethylcarbodiimide HCl, eq is equivalent(s), ES-MS and ESI-MS are electron spray ion-mass spectroscopy, Et is ethyl, Et₂O is diethyl ether, EtOH is ethanol, EtOAc is ethyl acetate, h or hr is hour(s), HATU is O-(7-azabenzotriazol-1-yl)-1,1,3,3-tetramethyluronium hexafluorophosphate, Hex is hexane, HMPA is hexamethyl phosphoramide, HOAt is 1-hydroxy-7-azabenzotriazole, HOBt or HOBT is 1-hydroxybenzotriazole hydrate, HPLC is high performance liquid chromatography, LC-MS or LC-MASS is liquid chromatography mass spectrum, LDA is lithium diisopropylamide, LG is leaving group, LiHMDS is lithium hexamethyl disilazide, MC-xR is melanocortin receptor (x being a number), Me is methyl, MF is molecular formula, mL is milliliter, mmol is millimole(s), min is minute, MPLC is medium pressure liquid chromatography, MS is mass spectrum, Ms is methane sulfonyl, MeOH is methanol, MTBE is tert-butyl methyl ether, NMM is N-Methylmorpholine, NMO is N-Methylmorpholine-N-oxide, OTf is trifluoromethanesulfonyl, Ph is phenyl, Phe is phenyl alanine, Pr is propyl, iPr is isopropyl, prep. is prepared, PyBOP is benzotriazol-1-yloxytripyrrolidine-phosphonium hexafluorophosphate, PyBrop is bromo-tris-pyrrolidino-phosphonium hexafluoro-phosphate; r.t., RT or rt is room temperature, SCF CO₂ S is super critical fluid carbon dioxide, TBDMS is tert butyl dimethyl silyl, TEA is triethylamine, Tf is triflate or trifluoromethanesulfonate, Tf₂O is triflic anhydride, TFA is trifluoroacetic acid, THF is tetrahydrofuran, and TLC is thin-layer chromatography.

Reaction Schemes A-O illustrate the methods employed in the synthesis of the compounds of the present invention of structural formula I. All substituents are as defined above unless indicated otherwise.

The synthesis of the novel compounds of structural formula I which are the subject of this invention may be accomplished by one or more of several similar routes. In all cases it is necessary to effect an amide bond coupling between a substituted piperidine of general formula 1 and a cycloalkyl carboxylic acid derivative of general formula 2 as shown in reaction Schemes A-C below. Once the amide bond coupling reaction is accomplished, it may be necessary to further synthetically modify the coupled product to incorporate the desired substituents on the cycloalkyl carboxylic acid ring or to remove protecting groups. Reaction Scheme A illustrates the synthetic methodology in the most general case where a cycloalkyl carboxylic acid derivative 2 bearing the desired R¹ substituent is coupled with a substituted piperidine of general formula 1 to afford an amide corresponding to the compounds of structural formula I. The amide bond coupling reaction illustrated in reaction Scheme A is conducted in an appropriate inert solvent such as methylene chloride, dimethylformamide, or the like and may be performed with a variety of reagents suitable for amide coupling reactions such as EDC or PyBOP. Preferred conditions for the amide bond coupling reaction shown in reaction Scheme A are known to those skilled in organic synthesis. Such modifications may include, but are not limited to, the use of basic reagents such as TEA or NMM, or the addition of an additive such as HOBt. Alternatively, 4-substituted piperidines of formula 1 may be treated with an active ester or acid chloride derived from carboxylic acid 2 which also affords compounds of structural formula I. The amide bond coupling shown in reaction Scheme A is usually conducted at temperatures between 0° C. and room temperature, occasionally at elevated temperatures, and the coupling reaction is typically conducted for periods of 1 to 24 hours.

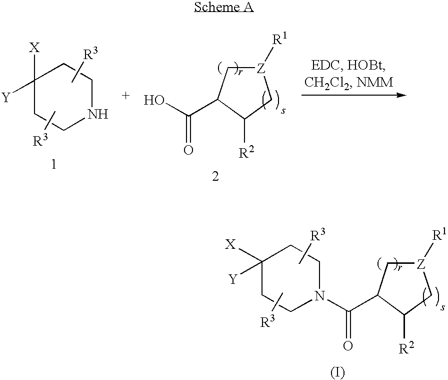

Reaction Schemes B and C illustrate the synthesis of the novel compounds of structural formula I when it is preferred to affect the amide bond coupling step prior to incorporation of the basic substituent R¹ as mentioned above. Reaction Scheme B illustrates a preferred method for the synthesis of compounds of structural formula I which employs a piperidine of general formula 1 and a cycloalkanone carboxylic acid of general formula 3 as the partners in the amide bond coupling step. The piperidine of formula 1 and the carboxylic acid of formula 3 are first coupled to afford an amide of general formula 4 using the reagents and conditions described for the generalized amide coupling shown in reaction Scheme A. The R¹ substituent (R¹═NR⁹R⁷) may then be incorporated at the position of the carbonyl group by performing a reductive amination reaction with an amine of general formula 5. Typical conditions for effecting such a reductive amination include performing an imine 6 from ketone 3 and amine 5 followed by reduction of the intermediate imine with reducing agents such as sodium borohydride, sodium cyanoborohydride or sodium triacetoxyborohydride. Formation of the intermediate imine 6 derived from piperidine 1 and acid 3 may occur spontaneously in solution or it may be promoted with agents such as titanium (IV) isopropoxide in a solvent such as methanol or with anhydrous magnesium sulfate in chloroform. The formation of the imine 6 is generally performed at temperatures between 0° C. and the reflux temperature of the solvent being used, frequently at room temperature. The imine formation step is generally allowed to proceed to completion over a period of several hours to 1 day prior to the reduction step which minimizes the formation of secondary alcohols formed by simple reduction of the keto group in compounds of general formula 4. The intermediate imine 6 may in some cases be isolated and purified, however it is generally preferred to use it directly in the reduction step. The reduction of the imine 6 is typically conducted in an alcoholic solvent such as methanol or ethanol at temperatures between 0° C. and room temperature, and the reduction is generally completed in periods of several hours or less.

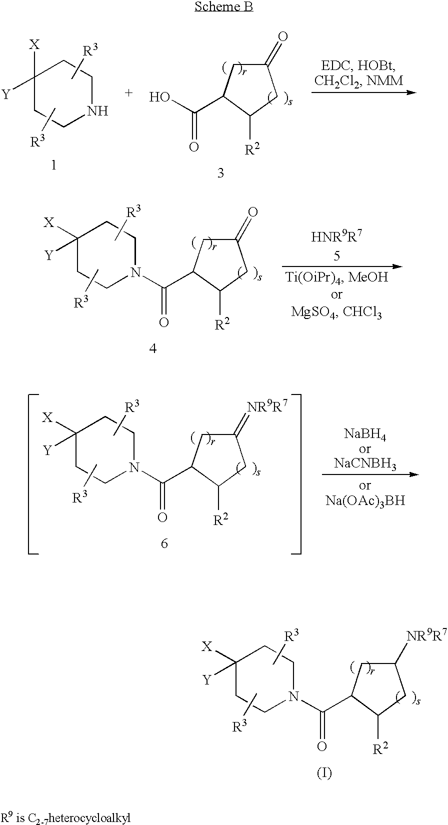

Reaction Scheme C illustrates a preferred method for the synthesis of compounds of structural formula I which employs a piperidine of general formula 1 and a hydroxyl-substituted cycloalkyl carboxylic acid of general formula 7 as the partners in the amide bond coupling step. The amide bond coupling step between piperidine 1 and carboxylic acid 7 is performed first, typically using a carbodiimide reagent like EDC to promote the coupling as described above. The hydroxyl-substituted amide 8 which is produced is then further synthetically modified to incorporate the R¹ substituent present in the compounds of structural formula I. A variety of methods known to those skilled in organic synthesis may be used to incorporate the R¹ substituent. For instance, the hydroxyl group of compounds of general formula 8 may be oxidized using a variety of methods to afford carbonyl compounds of general formula 4. The resulting ketoamides of general formula 4 may then be converted to the compounds of structural formula I using the reductive amination reaction described in reaction Scheme B.

Occasionally, it may be preferable to utilize hydroxyl-substituted compounds of general formula 8 in a Fukuyama-Mitsunobu reaction (Fukuyama, T.; Cheung, M.; Jow, C.-K.; Hidai, Y.; Kan, T. Tetrahedron Lett. 1997, 33, 5831-4) sequence as shown in reaction Scheme C. In this method for the synthesis of the compounds of structural formula I, the intermediate hydroxyl-substituted cycloalkylamide 8 is reacted with a 2,4-dinitrobenzenesulfonamide of general formula 9 in the presence of triphenylphosphine and an azodicarboxylate reagent such as DEAD. The reaction is performed in a suitable aprotic solvent such as benzene, toluene or tetrahydrofuran, typically at room temperature, and the reaction is generally complete in 0.5-3 hours. The product of this reaction is the secondary 2,4-dintrobenzenesulfonamide of general formula 10, which may then be readily converted to a compound of structural formula I wherein R⁷═H. The deprotection of the sulfonamide group is accomplished by reaction of 10 with either a base like n-propylamine in a solvent like methylene chloride or by reaction of 10 with a nucleophilic reagent such as mercaptoacetic acid with triethylamine in methylene chloride. In either case the reaction is typically conducted at room temperature, for periods of 5 minutes to one hour. An advantage of the Fukuyama-Mitsunobu reaction sequence is that the stereochemistry of the carbon atom undergoing substitution is cleanly inverted. Thus if the hydroxyl-substituted cycloalkylamide 8 is a single diastereoisomer, then the product 10 will be a single diastereoisomer also. This is in contrast to the reductive amination strategy discussed in reaction Scheme B which generally affords a mixture of epimeric products.

The secondary amine of formula I (R⁷=H) shown in reaction Scheme C may then be further synthetically modified using a variety of methods known in organic synthesis to incorporate other embodiments of the R⁷ substituent. For instance, compounds of structural formula I where R⁷═H may be subjected to a reductive amination reaction with an appropriate aldehyde or ketone using the conditions described in reaction Scheme B.

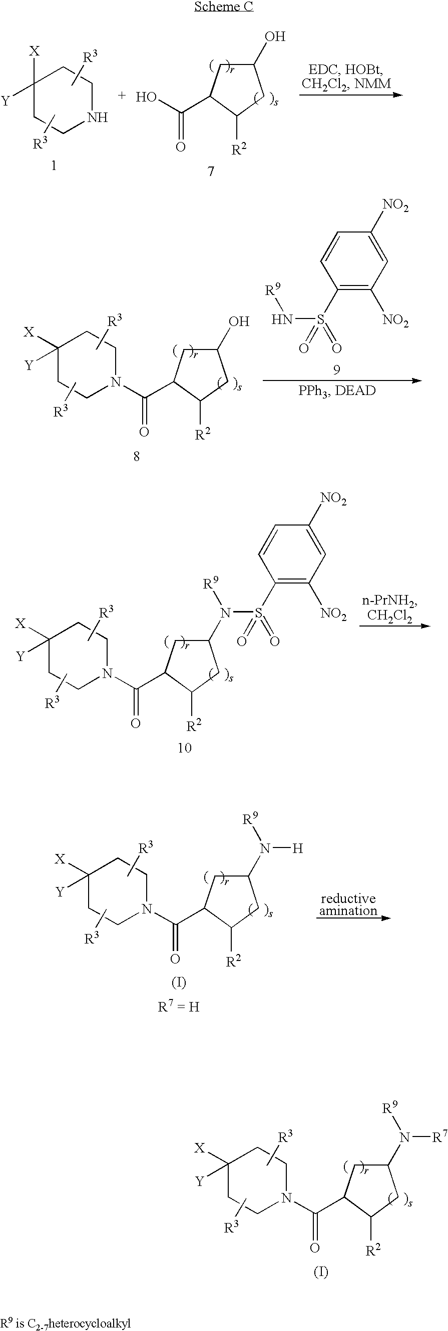

Reaction Scheme D illustrates a preferred method for the synthesis of the cycloalkyl carboxylic acids of general formula 3 when the values of r and s are selected such that the resulting carbocyclic ring is a six-membered ring. In this method a Diels-Alder reaction between an α,β-unsaturated ester of general formula 11 and 2-trimethylsilyloxybutadiene 12 affords a mixture of the two regioisomeric silylenolethers 13 and 14. The silylenolethers 13 and 14 are generally subjected to an hydrolysis reaction using hydrochloric acid in a solvent such as methanol and the two regioisomeric ketones 15 and 16 are then separated by conventional chromatographic methods. The olefin geometry of the starting α,β-unsaturated ester of general formula 11 determines the relative stereochemistry of the two substituents on the six-membered ring. Thus a trans α,β-unsaturated ester 11 affords the trans-disubstituted products 13 and 14 as shown, whereas the corresponding cis isomer of compounds of general formula 11 will afford the corresponding cis isomers of 13 and 14. Once the regioisomeric cyclohexanones of general formulae 15 and 16 are separated, they may then be individually hydrolyzed. For instance, hydrolysis using lithium hydroxide in refluxing tetrahydrofuran, affords the carboxylic acids of general formula 3 (r=2, s=1) and 3 (r=1, s=2). The acids of general formula 3 are finally converted to the compounds of structural formula I using the methodology described above in reaction Scheme B.

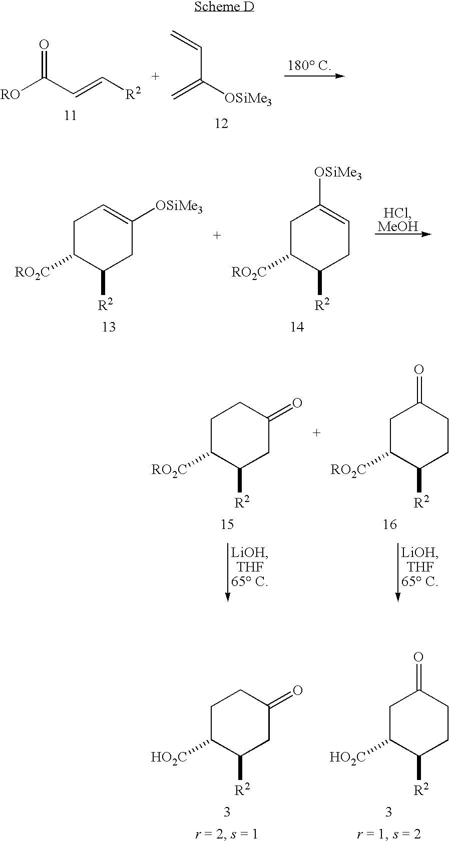

Reaction Scheme E illustrates a preferred method for the synthesis of the cycloalkyl carboxylic acids of formula 3, which correspond to acids of general formula 2 wherein the values of r and s are selected such that the resulting carbocyclic ring is a five-membered ring. In this method an α,β-unsaturated ester of general formula 11 is subjected to a trimethylenemethane cycloaddition reaction (Trost, B. M.; Chan, D. M. T. J. Am. Chem. Soc. 1979, 101, 6429) to afford a cyclopentane derivative of general formula 18. The cycloaddition is performed by reacting the α,β-unsaturated ester of general formula 11 with 2-[(trimethylsilyl)methyl]-2-propen-1-yl acetate 17 in the presence of a palladium (0) catalyst in a solvent such as tetrahydrofuran. A preferred palladium (0) catalyst for the cycloaddition may be generated by mixing palladium acetate and triisopropyl phosphite in the reaction mixture. The cycloaddition reaction is typically conducted at the reflux temperature of the solvent, for instance 65° C., and the reaction is usually completed in periods of 2-8 hours. The olefin geometry of the starting α,β-unsaturated ester of general formula 11 determines the relative stereochemistry of the two substituents on the five-membered ring. Thus a trans α,β-unsaturated ester 11 affords the trans-disubstituted product 18 as shown, whereas the corresponding cis isomer of compounds of general formula 11 affords the corresponding cis-disubstituted isomer of 18. The exocyclic olefin present in compounds of general formula 18 is next oxidatively removed to afford a cyclopentanone derivative of general formula 19. A preferred method for the oxidative cleavage reaction is the two step process shown at the bottom of reaction Scheme E. The methylene cyclopentane derivative of formula 18 is first oxidized to a 1,2-diol derivative using catalytic osmium tetraoxide in the presence of a stoichiometric reoxidant such as N-methylmorpholine-N-oxide and a solvent system such as acetone-water. The intermediate 1,2-diol which forms is generally not isolated, but is in turn subjected to cleavage with sodium periodate in a solvent system like methanol-water to afford ketones of general formula 19. Both steps in the oxidative cleavage sequence are generally completed during periods of several minutes to a few hours and the reaction steps are typically conducted at low temperatures, for instance between 0° C. and room temperature. Alternatively, the oxidative cleavage of olefins of general formula 18 may be accomplished using ozone, or by other methods known in organic synthesis. The cyclopentanones of general formula 19 may then be hydrolyzed, for instance using sodium hydroxide in methanol, to afford the carboxylic acids of formula 3 (or general formula 2 wherein r=1, s=1). The acids of general formula 3 are finally converted to the compounds of structural formula I using the methodology described above in reaction Scheme B.

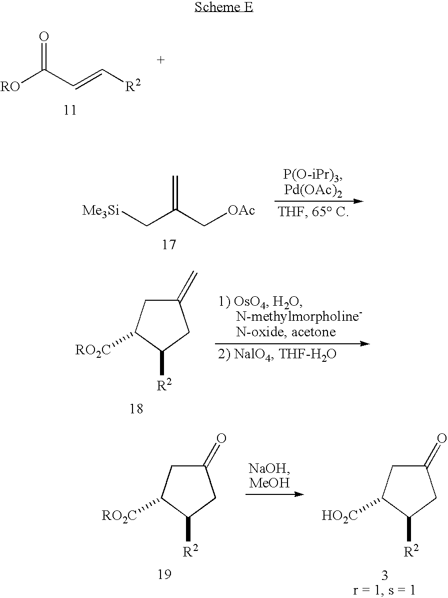

When it is desired to prepare individual enantiomers of the compounds of structural formula I, it is possible to perform a resolution of the compounds of structural formula I using one of the methods known in the art of organic synthesis. For instance, enantiomerically pure compounds (I) may be prepared by crystallization of diastereoisomeric salts formed from the racemic compounds of structural formula I and an optically active carboxylic acid. The two diastereoisomeric salts are separated from each other by fractional crystallization, then the enantiomerically pure compounds of structural formula I are regenerated by treatment of the purified salts with a base. Alternatively, racemic compounds of structural formula I may be resolved by preparative HPLC using commercially available chiral-stationary phase columns. Another strategy for the preparation of enantiomerically pure compounds of structural formula I involves preparing enantiomerically pure compounds of general formula 2 prior to their use in the amide bond forming reaction outlined in reaction Scheme A. Racemic compounds of general formula 2, or intermediates used to prepare compounds of formula 2 as described in the previous reaction Schemes (i.e. acids 3 and 7, or esters 15, 16 and 19) may also be resolved using the classical methods previously discussed.

Reaction Scheme F illustrates a strategy for the synthesis of pyrrolidine acids of general formula 2. The preferred method for the synthesis of compounds of general formula 2 involves the azomethine ylid 3+2 cycloaddition reaction of an azomethine ylid precursor of general formula 21 and a substituted cinnamic ester 20. The azomethine cycloaddition reaction of 20 and 21 affords the 3,4-disubstituted pyrrolidine 22, and the stereochemical relationship of the substituents on the newly formed pyrrolidine ring is determined by the stereochemistry of the double bond in the cinnamate ester 20. Thus the trans ester 20 affords a trans 3,4-disubstituted pyrrolidine of formula 22 as shown. The corresponding cis cinnamate ester affords a cis 3,4-disubstituted pyrrolidine of general formula 22. Cis or trans 3-arylpyrrolidine-4-carboxylic esters of general formula 22 may be resolved to afford enantiomerically pure compounds using a method such as resolution by crystallization of the diastereoisomeric salts derived from 22 and a chiral carboxylic acid, or directly by the use of chiral stationary phase liquid chromatography columns. Reaction Scheme F illustrates the case where a trans cinnamic ester 20 is converted to a trans 3,4-disubstituted pyrrolidine 22 and its subsequent resolution affords the enantiomerically pure trans pyrrolidine esters 23 and 24. Finally, the esters of general formula 22 (or their pure enantiomers 23 and 24) are hydrolyzed to the corresponding amino acid hydrochlorides of general formula 25 as shown at the bottom of reaction Scheme F.

Amino acids of general formula 25 are zwitterionic. Therefore it is in some cases difficult to achieve efficient separation and purification of these compounds from aqueous reactions or workups. In these cases it is preferred to affect the hydrolysis using a reagent such potassium trimethylsilanolate in diethyl ether. Under these conditions the potassium salt of the carboxylic acid is produced which affords an easily isolated precipitate in ether. The resulting salt is then converted to the corresponding amino acid hydrochloride by treatment with excess hydrogen chloride in a suitable solvent such as ethyl acetate. Alternatively, esters such as 22 may be converted directly to the amino acid hydrochlorides 25 under acidic hydrolysis conditions. The hydrolysis of the ester 22 is achieved by prolonged reaction with concentrated hydrochloric acid at an elevated temperature. For example, this reaction may be conducted in 8 M hydrochloric acid at reflux overnight. The reaction mixture is then cooled and evaporated in vacuo to afford the amino acid hydrochloride 25. The amino acid hydrochlorides of general formula 25 correspond to an amino acid hydrochloride of general formula 2 and may be employed directly in the amide bond coupling step illustrated in reaction Scheme A to produce the compounds of structural formula I.

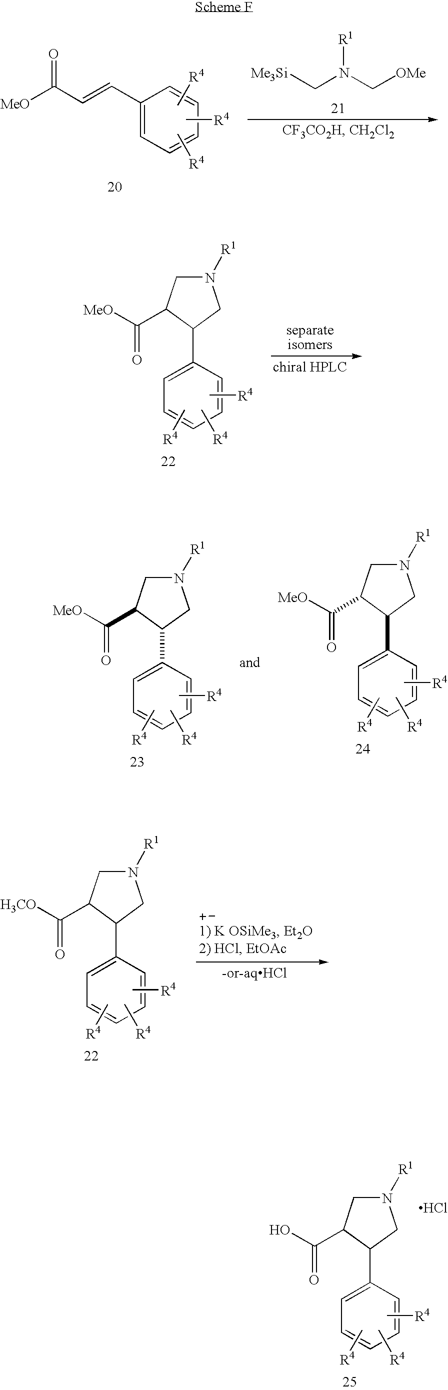

Another preferred method for the synthesis of enantiomerically pure 3-arylpyrrolidine-4-carboxylic acid derivatives is illustrated in reaction Scheme G. In this synthetic method, a substituted cinnamic acid of general formula 26 is first derivatized with a chiral auxillary such as (S)-(−)-4-benzyl-2-oxazolidinone 27. The acylation of chiral auxiliary 30 with cinnamic acids of formula 26 is performed by initial activation of the acid to afford a mixed anhydride. Typically acids of general formula 26 are reacted with an acid chloride such as pivaloyl chloride in the presence of a base such as triethylamine and in a suitable aprotic solvent such as THF. The intermediate cinnamyl-pivaloyl anhydride is converted to the product 28 by reaction with the oxazolidinone 27 in the presence of lithium chloride, an amine base such as triethylamine and in a solvent such as THF, and the reaction is conducted at temperatures between −20° C. and room temperature for periods of 1-24 hours. Alternatively, the oxazolidinone 27 may be deprotonated with a strong base such as n-butyllithium in THF at low temperatures such as −78° C. and then reacted with a mixed anhydride obtained from acid 26 and an acid chloride like pivaloyl chloride as noted above. The cinnamyl oxazolidinone of general formula 28, which is produced by either of these methods, is then reacted with the azomethine ylid precursor 21, and the products of the reaction are the substituted pyrrolidines of general formulas 30 and 31 as shown. The products 30 and 31 are diastereoisomers of each other and may therefore be separated by standard methods such as recrystallization or by liquid chromatography on a solid support such as silica gel. As discussed above, if the trans isomer of the cinnamic acid of general formula 26 is employed in the first step of reaction Scheme G, then a trans isomer of the substituted cinnamyl oxazolidinone 28 is produced. If such a trans cinnamyl oxazolidinone is then subjected to the azomethine ylid cycloaddition with an azomethine ylid precursor of formula 21, the products are the diastereoisomeric trans-disubstituted pyrrolidines related to 30 and 31.

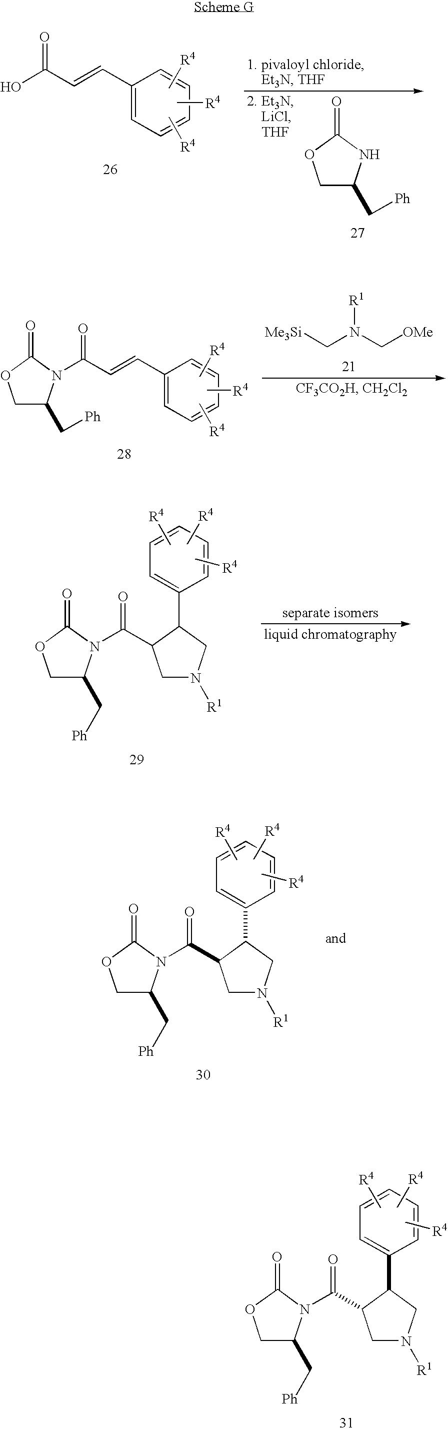

The azomethine ylid cycloaddition reactions shown in reaction Schemes F and G are generally conducted with the commercially available azomethine ylid precursor N-(methoxymethyl)-N-(trimethyl-silylmethyl)benzylamine (21, R¹=—CH₂Ph). When the R¹ substituent in the compounds of structural formula I is chosen to be a group other than benzyl, it is generally preferable to remove the benzyl group from the substituted pyrrolidine compound at this point, and replace it with a more readily removed protecting group such as an N—BOC group. Reaction Scheme H illustrates this process with a generalized 3,4-disubstituted pyrrolidine of formula 32. The preferred method for removal of the N-benzyl group from compounds of general formula 32 will depend upon the identity of the R³ substituents. If these substituents are unaffected by hydrogenation conditions, then the N-benzyl group may be removed by hydrogenolysis using a palladium on carbon catalyst in a solvent such as ethanol and in the presence of hydrogen gas or a hydrogen donor such as formic acid. Occasionally it may be preferred that one of the substituents R³ be a halogen or another substituent defined above which would be reactive under hydrogenation conditions. In these cases, the compound of general formula 32 is reacted with 1-chloroethyl chloroformate in an inert solvent such as toluene at temperatures between room temperature and 110° C. (Olafson, R. A. et al. J. Org. Chem. 1984, 49, 2081). The toluene is then removed, and the residue is heated in methanol for a period of 15-60 minutes, and the product is the debenzylated pyrrolidine of general formula 33. The resulting pyrrolidine 33 is then protected as its tert-butyl carbamate 34 using BOC anhydride in the presence of a base and a suitable solvent. For example, this can be accomplished in a two phase mixture of chloroform and aqueous sodium bicarbonate as shown in reaction Scheme H. The oxazolidinone chiral auxillary is next hydrolyzed from the pyrrolidines of general formula 34 as shown at the bottom of reaction Scheme H. The hydrolysis reaction is accomplished using lithium hydroperoxide generated in situ from lithium hydroxide and 30% aqueous hydrogen peroxide. The reaction is typically conducted in a solvent system such as aqueous THF, and the reaction is performed at temperatures between 0° C. and room temperature for a period of 1-6 hours. The resulting carboxylic acids of general formula 35 correspond to carboxylic acids of general formula 2. Using the methodology presented in reaction Scheme A, the compounds of general formula 35 may then be converted to the compounds of the present invention of structural formula (I).

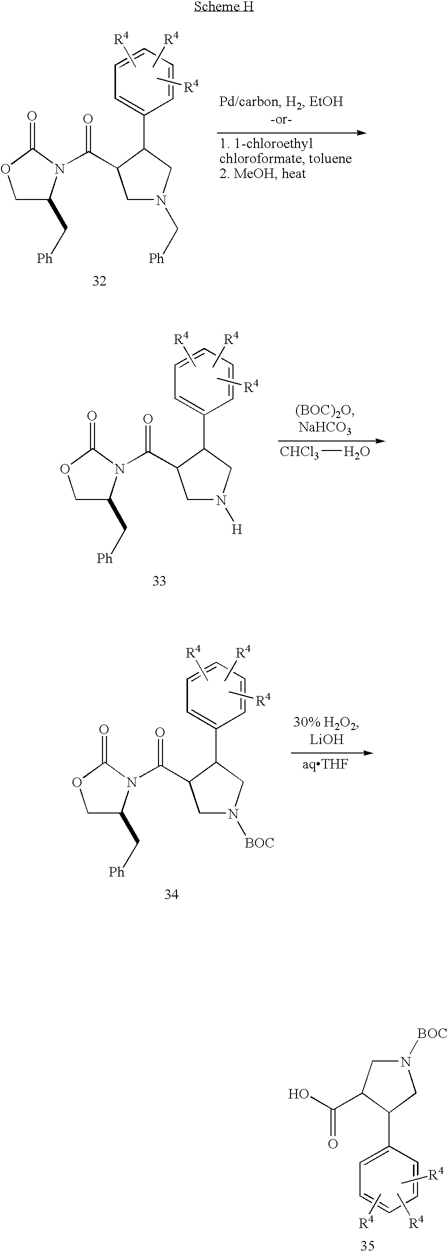

As noted previously in the discussion of reaction Scheme F, it may occasionally be preferable to incorporate the R¹ substituent into the substituted pyrrolidine of general formula 35 at an earlier stage of the synthesis, for instance when it is desired that R¹ be a tert-butyl group. In such cases, it is possible to utilize an azomethine ylid precursor 21 bearing the desired R¹ substituent in the cycloaddition reactions illustrated in reaction Schemes F and G. Reaction Scheme I illustrates the preparation of azomethine precursors of formula 21 starting with amines of general formula 36. Reaction of the amine of formula 36 with chloromethyltrimethylsilane at high temperature and in the absence of solvent affords the N-trimethylsilylmethyl-substituted amine of general formula 37. Subsequent reaction of 37 with aqueous formaldehyde in the presence of methanol and a base such as potassium carbonate then affords the generalized ylid precursor 21 which can be utilized in the cycloaddition reactions discussed above.

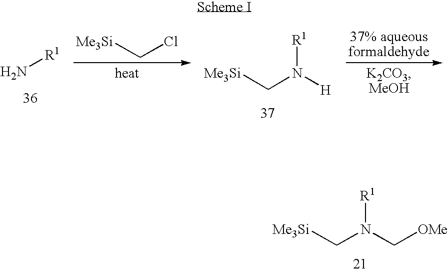

Reaction Scheme J illustrates a preferred method for the synthesis of compounds of general formula 2 wherein Z is a nitrogen, r is 2 and s is 1 such that the resulting heterocycle is a 3-aryl-4-piperidine carboxylic acid derivative 44 (n=1); and the synthesis of compounds of formula 2 wherein Z is a nitrogen, r is 1 and s is 1 such that the resulting heterocycle is a 3-aryl-4-piperidine carboxylic acid derivative 47 (n=2). The synthesis of 44 and 47 begins with a commercially available substituted benzene 38, such as difluorobenzene, which is derivatized to give the chloro ketone 39 via treatment with aluminum chloride and chloroacetylchloride. The ketone of 39 is reduced to the alcohol 40 using a borane N,N diethylaniline complex and a solution of (S)-2-methyl-CBS oxazaborolidine in MTBE, and the chlorine is displaced by R¹NH₂, for instance tert-butyl amine to give 41. The secondary amine nitrogen of 41 is alkylated with 4-bromo butyl nitrile (n=2) or 3-bromo propyl nitrile (n=1) to give nitrile compounds 42 and 45, which may be cyclized to the piperidine 46 and pyrrolidine 43 by treatment with LiHMDS and diethylphosphoryl chloride. Treatment of the nitrites 43 and 46 with sodium hydroxide provides the amides, which are subsequently converted to the corresponding methyl esters using HCl/MeOH and acetyl chloride, and to acids 44 and 47 by treatment with concentrated HCl. The resulting pyrrolidine acid 44 and piperidine acid 47 may be utilized in the coupling reaction shown in Scheme A.

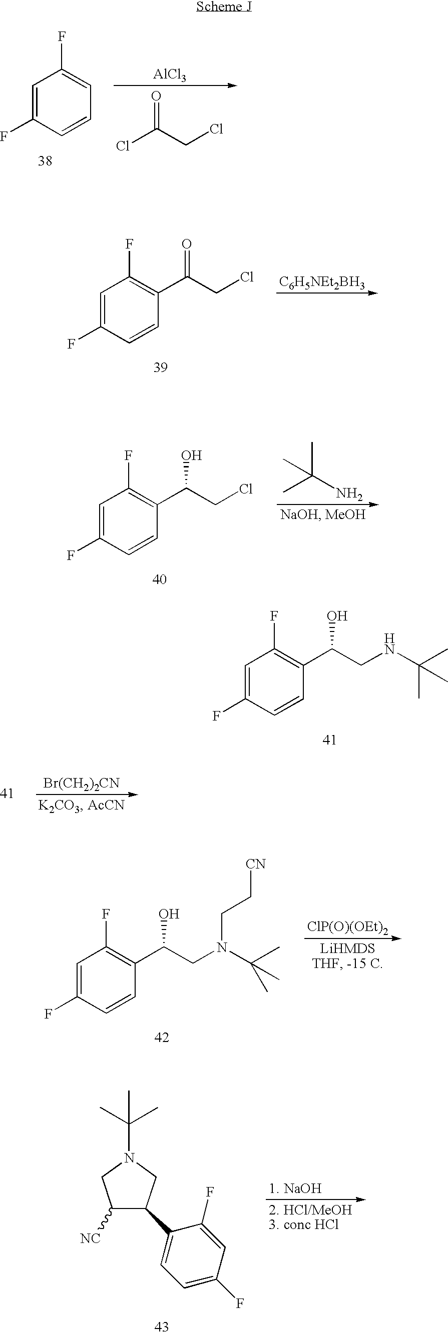

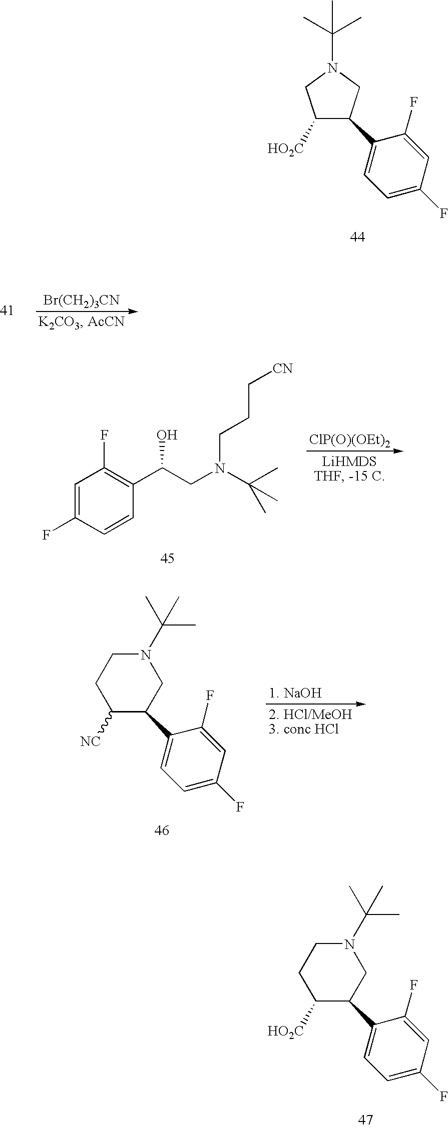

Reaction Scheme K illustrates a preferred method for the synthesis of compounds of general formula 2 wherein Z is a nitrogen, r is 1 and s is 2, such that the resulting heterocycle is a 4-aryl-3-piperidine-carboxylic acid derivative 54. The synthesis of 54 is similar to the synthesis shown in reaction Scheme J, and may begin with either of the commercially available β-keto esters 48 or 49. Conversion of 48 or 49 to the N-BOC-protected piperidine 50 is performed as shown and the resulting β-keto ester is subjected to the two-step arylation protocol previously described in Scheme J to yield 52. Reduction of the double bond of 52 using conditions appropriate for obtaining either cis or trans 53 is followed by ester hydrolysis which affords either a cis or trans 4-aryl-3-piperidine-carboxylic acid of general formula 54 which corresponds to an acid of general formula 2 wherein Z is a nitrogen, r is 1 and s is 2. The cis or trans carboxylic acids of general formula 54 are produced as racemates and either may be resolved to afford enantiomerically pure compounds by methods known in organic synthesis. Preferred methods include resolution by crystallization of diastereoisomeric salts derived from the acids 54 and a chiral amine base or by the use of chiral stationary phase liquid chromatography columns. As before, the cis or trans carboxylic esters 53 can also be resolved by the use of chiral stationary phase liquid chromatography columns.

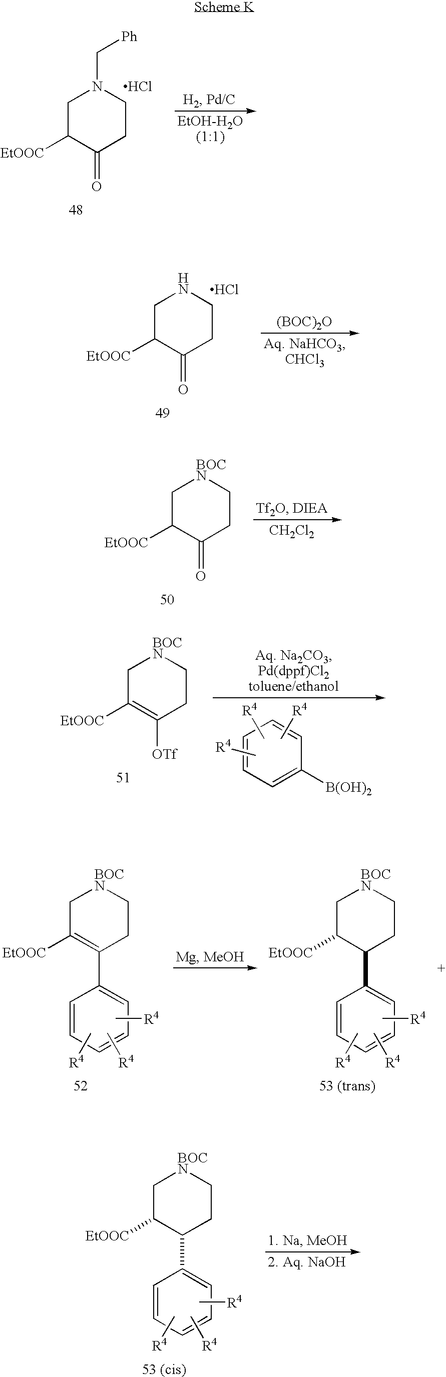

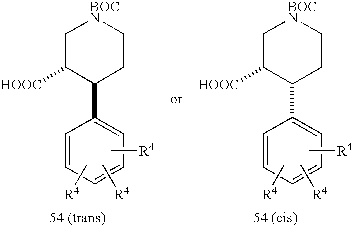

The synthesis of the N—BOC protected carboxylic acids of general formula 54 illustrated in reaction Scheme K is useful for the preparation of compounds of structural formula I (Z=N) bearing a variety of R¹ substituents as noted above. For the synthesis of certain compounds of structural formula I, for instance when it is desired that Z is nitrogen and R¹ is tert-butyl group, it is preferable to incorporate that R¹ substituent at an earlier stage of the synthesis. When it is desirable to synthesize a compound of general formula 54 wherein the BOC group is replaced with a substituent group R¹, a reaction sequence similar to the one illustrated in reaction Scheme K may be employed starting with a compound of general formula 50, which may be synthesized as shown in reaction Scheme L. An amine 55 bearing the desired R¹ substituent is first subjected to a Michael addition with excess ethyl acrylate in the presence of a solvent such as THF or ethanol. The resulting diester 56 is then converted to a 1-substituted-4-ketopiperidine-3-carboxylic ester 57 using an intramolecular Dieckmann reaction. The substituted piperidine 57 corresponds to a compound of general formula 50 shown in reaction Scheme K, wherein the BOC group is replaced with the desired R¹ substituent. The compounds of general formula 57 may then be converted to compounds of general formula 2 where the R¹ substituent replaces the BOC group using the methodology illustrated in reaction Scheme K.

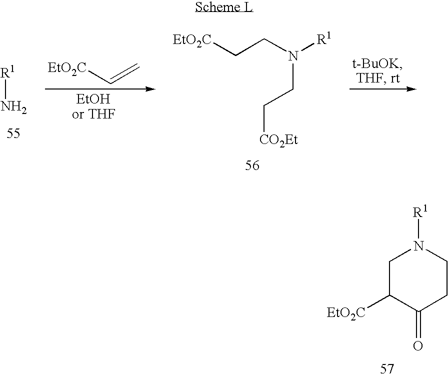

Reaction Schemes M, N and O illustrate additional methods for the synthesis of the 4-substituted piperidines of general formula 1 which are required in the amide bond coupling step illustrated in reaction Scheme A.

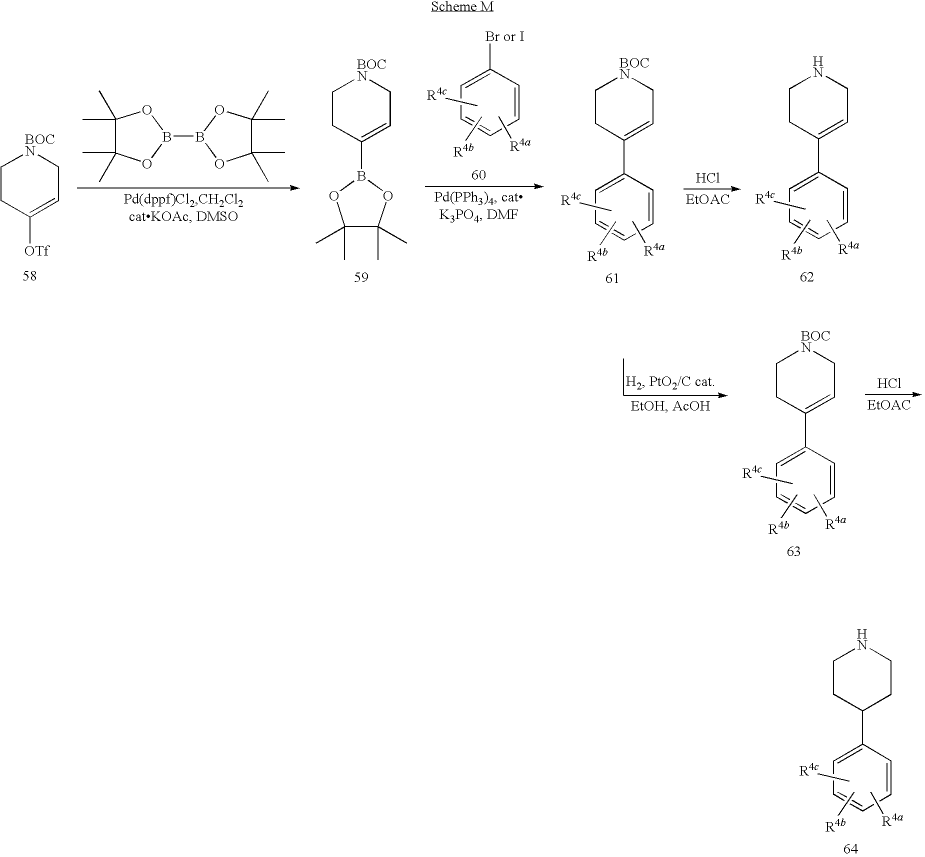

As shown in Reaction Scheme M, treatment of enoltriflate 58 (prepared as described in: Rohr, M.; Chayer, S.; Garrido, F.; Mann, A.; Taddei, M.; Wermuth, C-G. Heterocycles 1996, 43, 2131-2138.) with bis(pinacolato)diboron reagent in the presence of a suitable palladium (II) catalyst such as [1,1′-bis(diphenylphosphino)-ferrocene]dichloro-palladium (II) (Pd(dppf)Cl₂) and potassium acetate in a polar, inert organic solvent such as methyl sulfoxide at about 80° C. under an inert atmosphere for a period of 6-24 hours provided the vinyl dioxaborolane 59. Borolane 59 can be further reacted with an aryl halide such as 60 in the presence of a palladium catalyst such as tetrakis(triphenylphosphine)palladium (0) (Pd(Ph₃)₄) and potassium phosphate in an inert solvent such as N,N-dimethylformamide to give the coupled 4-aryl tetrahydropyridine product 61. The tert-butyloxycarbonyl protecting group can be removed by any of the known methods such as treatment with a protic acid such as hydrogen chloride in an inert organic solvent such as ethyl acetate or trifluoroacetic acid in methylene chloride to give amine 62. Alternatively, it is sometimes desirable to reduce the double bond in synthetic intermediate 61. This can be effected by treatment with hydrogen at atmospheric or elevated pressure and a noble metal catalyst on carbon such as palladium (0) or platinum(IV) oxide in an inert organic solvent such as ethanol, ethyl acetate, acetic acid or mixtures thereof to give the 4-arylpiperidine 63. Removal of the tert-butyloxycarbonyl protecting group as described above provides amine 64. Both amine intermediates, 62 and 64, may be used as coupling partners in Reaction Scheme A.

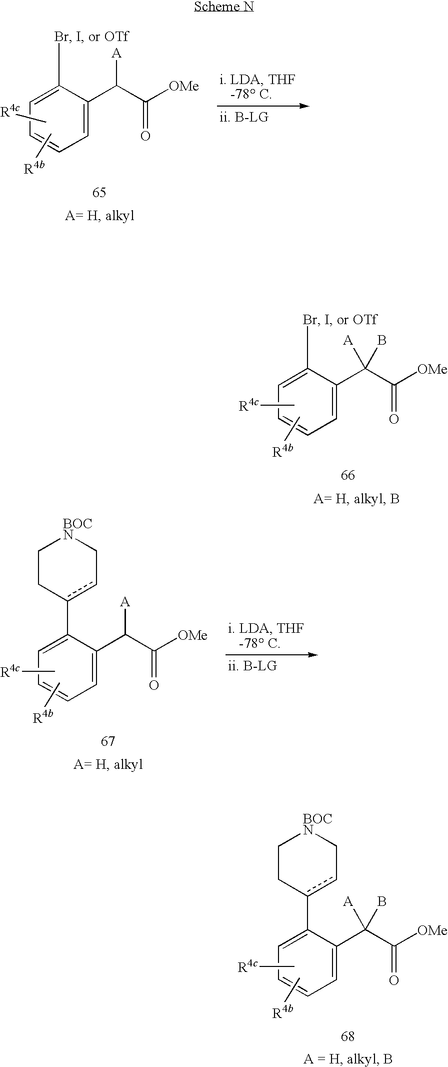

As shown in Reaction Scheme N, aryl groups containing substituents with acidic hydrogens (e.g. 65 and 67) can modified by alkylation under known protocols. For instance, treatment of esters 65 or 67 with a strong base such a lithium diisopropylamide at low temperature in an inert organic solvent such as tetrahydrofuran can form an intermediate enolate which can be reacted in a second step with any alkylating agent (B-LG) such as iodomethane, iodoethane, 1,2-dibromoethane or the like to form the corresponding alkylated product. In addition to ester groups, related amides and functionalities that promote the formation of a stable anion can be alkylated under similar protocols.

Ester intermediates such as 66 and 68 may be further modified by conversion to the corresponding carboxylic acids and coupled with amines to form amides as described in Reaction Scheme O. Conversion of the methyl esters 66 and 68 to the carboxylic acid can be effected by dealkylation using potassium trimethylsilanolate at room temperature in an inert organic solvent such as tetrahydrofuran for a period of about one to about 24 hours to provide, after acidification, the corresponding carboxylic acids. In certain cases, a base-catalyzed hydrolysis known to those skilled in the art may be used to effect this same transformation. These acids may be reacted further to form amides by treatment with a primary or secondary amine under a variety of amide coupling protocols such as described in Scheme A to provide intermediates 69 and 70.

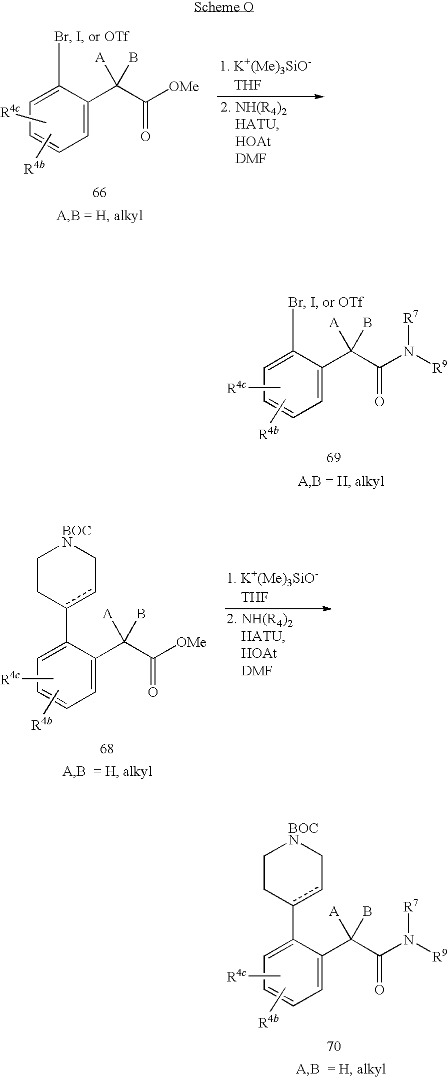

The preparation of other 4-substituted piperidine intermediates of general formula 1 for coupling with the carboxylic acids of general formula 2 as shown in Scheme A is disclosed in WO 00/74679 (14 Dec. 2000), which is incorporated by reference herein in its entirety. The preparation of additional 4-substituted piperidine intermediates needed to derive the compounds of formula I is provided below.

The following intermediates and examples are provided to illustrate the invention and are not to be construed as limiting the scope of the invention in any manner.

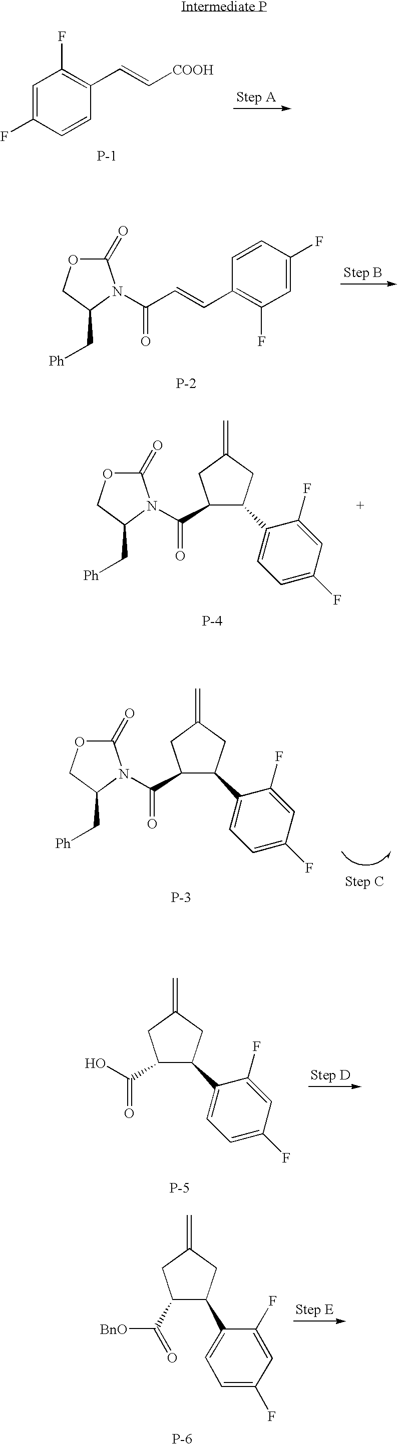

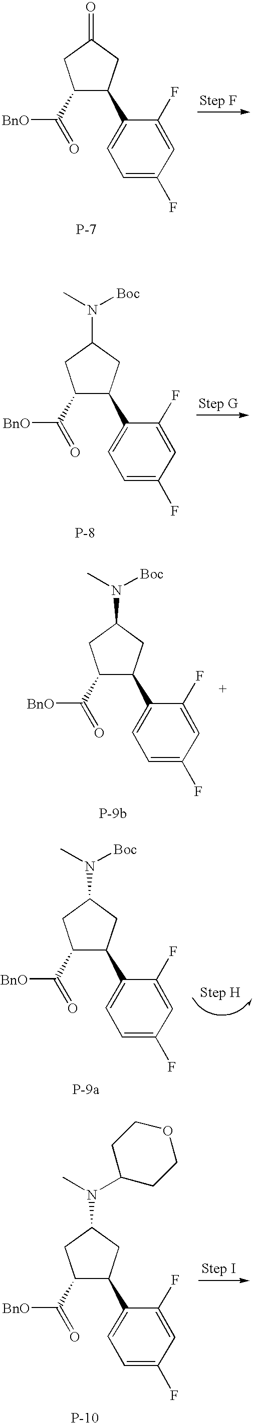

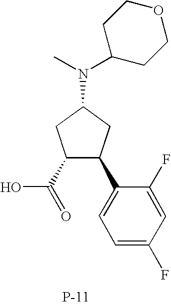

Step A: To a solution of trans-2,4-difluorocinnamic acid P-1 (7.6 g, 41.3 mmol, Aldrich) in THF (150 mL) was added triethylamine (17.3 mL, 123.8 mmol). The reaction mixture was cooled to −40° C. and trimethyl acetic chloride (5.1 mL, 47.3 mmol) was added slowly. After the reaction mixture was stirred at −40° C. for another 2 hours, the lithium chloride (1.93 g, 45.40 mmol) was added, followed by s-4-benzyl-2-oxazolidinone (7.31 g, 41.3 mmol). After stirring at −40° C. for another 20 min., reaction mixture was allowed to warm up to room temperature and stirred at r.t. for 18 hrs. The reaction mixture was poured into aqueous of saturated ammonium chloride (180 mL); the phases were separated and the aqueous phase was extracted with ethyl acetate. The combined ethyl acetate extracts were washed with brine, dried over MgSO₄ and concentrated to give a residue. The resulting residue was purified by crystallization from EtOAc/hexane to give compound P-2. ESI-MS calc. for C₁₉H₁₅F₂NO₃: 343; Found: 344 (M+H), 366 (M+Na).

Step B: To a solution of Compound P-2 (2.3 g, 6.55 mmol) in THF (30 mL) was added palladium acetate (73.6 mg, 0.33 mmol) and 2-[(trimethylsilyl)methyl]-2-propenol-yl acetate (1.8 mL, 8.52 mmol). The reaction vessel was evacuated under vacuum and purged with nitrogen 3 times, then triisopropyl phosphate (0.45 mL, 1.97 mmol) was added. The reaction mixture was heated at 65° C. for 18 hrs, cooled to r.t. and concentrated to give a residue. The resulting residue was partitioned between ethyl acetate and water, and the aqueous layer was extracted with ethyl acetate. The combined extracts were washed with brine, dried over MgSO₄ and concentrated to give a residue. The resulting residue was purified by HPFC (2-30% ethyl acetate in hexane) to give a yellow oil P-4 (0.89 g, fast elusion) and white solid P-3 (0.85 g, slow elusion). ESI-MS calc. for C₂₃H₂₁F₂NO₃: 397; Found: 398 (M+H), 420 (M+Na).

Step C: To a solution of Compound P-3 (1.7 g, 4.28 mmol) in THF (24 mL) and water (6 mL) under nitrogen at 0° C. was added lithium hydroxide monohydrate (0.36 g, 8.56 mmol) and H₂O₂ (30% solution, 2.5 mL, 25.7 mmol). After stirring the reaction mixture at 0° C. for half an hour, the mixture was warmed up to r.t. and stirred for 1.5 hours. The solvent was removed, the pH was adjusted to pH 9-10 with a saturated NaHCO₃ solution and the mixture was extracted with CH₂Cl₂. The aqueous layer was acidified with HCl (2N) to pH 1-2, and the mixture was extracted with CH₂Cl₂. The combined methylene chloride layers were dried over MgSO₄ and concentrated to give colorless oil P-5. ESI-MS calc. for C₁₃H₁₂F₂O₂: 238; Found: 239 (M+H).

Step D: To a solution of acid P-5 (6.05 g) in anhydrous CH₂Cl₂ (100 mL) was added Et₃N (4.1 mL). The reaction mixture was cooled to 0° C., then PhCH₂OCOC1 (1.05 eq., 3.7 mL) was added via a syringe dropwise under N₂. After stirring for 5 min at 0° C., solid DMAP (0.1 eq., 310 mg) was added and the reaction was stirred at 0° C. for 1 h. The reaction was quenched by ice, followed by NaHCO₃ (sat. aq.). The mixture was extracted with EtOAc/hexanes 3 times. The organic layer was separated, washed with brine, dried over Na₂SO₄ and concentrated to give the crude product P-6 (6.83 g), which was used in the next step without further purification.

Step E: Ester P-6 (25.4 mmol) was treated with t-BuOH (72 mL) followed by H₂O (24 mL) at room temperature. To this mixture was added OSO₄ (2.5% in t-BuOH, 3.2 mL) followed by NaIO₄ (13.6 g) at 2 min later at room temperature. After stirring 1.5 h at room temperature, the reaction mixture was filtered through celite and the solid was washed with EtOAc (3 times). The filtrate was washed with water and organic layer was separated, then washed with Na₂S₂O₃ (saturated aqueous) followed by brine. The aqueous layer was extracted with EtOAc. Organic layers were combined and washed with Na₂S₂O₃ (saturated aqueous) and brine. The combined organic layers were dried (Na₂SO₄) and concentrated to give the crude product P-7, which was used the next step without purification.

Step F: A mixture of crude ketone P-7 (25 mmol), molecular sieves (48 g, Aldrich catalog no 233668), MeNH₂.HCl (16.9 g) and Et₃N (70 mL) in CH₂Cl₂ (500 mL) was cooled to 0° C. Solid NaBH(OAc)₃ (53 g) was added. The bath was removed and the reaction was stirred at RT overnight. The reaction was filtered through celite. The solid was washed with cold 2 N NaOH (two times) followed by CH₂Cl₂ (two times). The CH₂Cl₂ layer was separated and the aqueous layer was extracted with CH₂Cl₂ (3 times). The combined CH₂Cl₂ layers were dried over Na₂SO₄ and concentrated to afford a residue, which was dissolved in CH₂Cl₂ (50 mL). The solution was treated with 2 N NaOH (aq, 20 mL) and Et₃N (14 mL, 100 mol, 4 eq.) followed Boc₂O (10.9 g) at 0° C. The bath was removed and the reaction was stirred at room temperature for 2 h. The reaction was diluted with water, CH₂Cl₂ layer was separated and the aqueous layer was extracted with CH₂Cl₂ (3 times). The combined CH₂Cl₂ layers were dried over Na₂SO₄ and concentrated to afford a residue, which was purified (2% EtOAc to 40% EtOAc in Hex) to afford a diastereomeric mixture P-8 (7.3 g, ratio ca. 2:1).

Step G: Compound P-8 was separated with prep Chiral HPLC to afford P-9a and P-9b. Analytical conditions: Chiral O J 4.6×250 mm 5u column, flow rate at 0.5 mL/min with 20% 2-propanol in heptane, and UV detection at 220 nm, t_{R}(S-4-a) 9.460 min, t_{R}(S-4-b) 14.460 min.

Step H: A solution of P-9a (3.75 g) in CH₂Cl₂ (5 mL) was treated with 4 N HCl in dioxane (30 mL). After 30 min, the mixture was concentrated to afford a residue, which was treated with molecular sieve (16 g, Aldrich catalog no 233668), Et₃N (23 mL), tetrahydro-4H-pyran-4-one (4.22 g) and CH₂Cl₂ (150 mL). To this mixture was added NaBH(OAc)₃ (17.9 g). The mixture was stirred at room temperature for 38 h, then worked-up analogous to the work up procedure of Step F. The resulting crude product was dissolved in CH₂Cl₂, and treated with Et₃N (4.7 mL), Boc₂O (1.84 g), and NaOH (1N, 20 mL) analogous to Step F. The work-up of this reaction was also analogous to Step F. The resulting crude product was purified by MPLC on silica gel (2% acetone in hexanes to 100% acetone) to give the product P-10.

Step I: A solution of P-10 (200 mg) in 2-propanol (2 mL) was treated with HCl (1M, 0.7 mL, 1.5 eq) followed by Pd/C (10%, 49 mg). The mixture was hydrogenated with a H₂ balloon overnight. The reaction was filtered and the filtrate was concentrated to afford P-11.

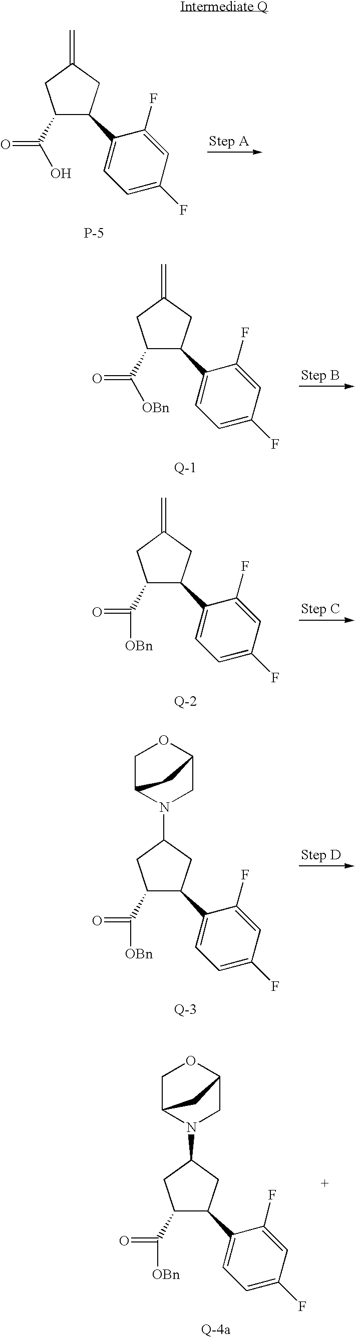

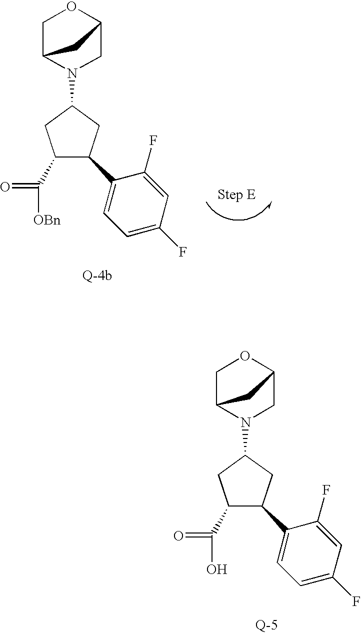

Step A: To the stirred solution of compound P-5 (2.4 g) in DMF (10 mL) was added Et₃N (1.4 mL), NaHCO₃ (2.57 g) and benzyl bromide (1.8 mL). The mixture was stirred at room temperature overnight, followed by partitioning between EtOAc and 1N HCl solution. The layers were separated and the aqueous layer was extracted with EtOAc three times. The organic phases were combined, dried over anhydrous MgSO₄, and purified by a flash column chromatography on silica gel (gradient elution: 0-20% EtOAc/Hexane as eluent) to give Q-1. ESI-MS calc. for C₂₀H₁₈F₂O₂: 328; Found: 329 (M+H).

Step B: To the stirred solution of compound Q-1 (2.97 g) in THF (100 mL) and H₂O (20 mL) was added dropwise a solution of OSO₄ in t-BuOH (11.3 mL, 2.5 wt % in t-BuOH). The mixture was stirred for 20 minutes, then a solution of NaIO₄ (7.73 g) in H₂O (80 mL) was added. The mixture was stirred at room temperature overnight, then quenched with addition of a saturated Na₂S₂O₃ solution (100 mL). EtOAc was added to the mixture to extract the product out three times. The organic phases were combined, dried over anhydrous MgSO₄ and concentrated in vacuo to give Q-2 as pale yellow solid, which was used in the next step without further purification. ESI-MS calc. for C₁₉H₁₆F₂O₃: 330; Found: 331 (M+H).

Step C: To the stirred solution of Q-2 (1.0 g) in CH₂Cl₂ (10 mL) was added (1S,4S)-2-oxa-5-azabicyclo[2.2.1]heptane HCl salt (1.23 g), DIPEA (1.58 mL) and molecular sieves (2 g). After stirring for 30 minutes, Na(OAc)₃BH (1.92 g) was added. The reaction suspension was stirred at room temperature overnight. After filtration, the filtrate was washed with saturated NaHCO₃, brine and concentrated. The resulting residue was purified by a flash column chromatography on silica gel to give a racemic mixture of Q-3. ESI-MS calc. for C₂₄H₂₅F₂NO₃: 413; Found: 414 (M+H).

Step D: The racemic mixture of compound Q-3 was resolved on high performance chromatography with ChiralPak OD column (Chiral Pak OD 10×250 mm 5u column, flow rate at 9 mL/min with 8% isopropanol in heptane, and UV detection at 220 nM) to afford two separate enantiomers Q-4a and Q-4-b.

Step E: To a solution of compound Q-4-a (450 mg) in EtOH (50 mL) was added Pd(OH)₂/C (400 mg). The mixture was stirred under a hydrogen atmosphere overnight. The solids were removed by filtration and the filtrate was concentrated in vacuo to give Q-5. ESI-MS calc. for C₁₇H₁₉F₂NO₃: 323; Found: 324 (M+H).

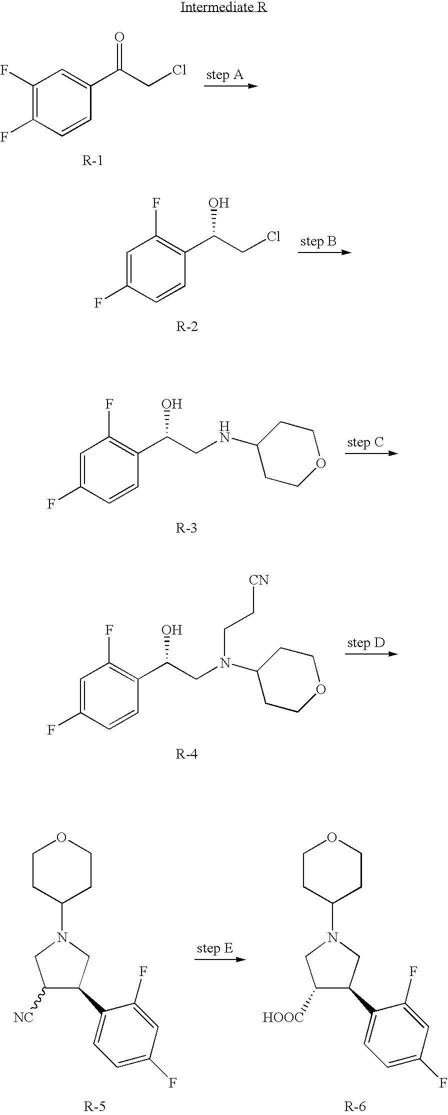

Step A: A solution of (S)-2-methyl-CBS-oxazaborolidine (0.26 mL, 1M in toluene), borane-N,N-diethylaniline (9.3 mL) in MTBE (20 mL) was heated to 40° C., then a solution of 2-chloro-2′,4′-di-fluoro-acetophenone R-1 (10 g) in MTBE (32 mL) was added over one hour. The homogeneous solution was stirred at 40° C. for one hour, then allowed to cool to room temperature and stirred overnight. The reaction mixture was then cooled to 0° C. and methanol (4.6 mL) was added slowly. The resulting mixture was stirred at room temperature for 30 minutes, then 2 N aqueous HCl (52.4 mL) was added slowly at 0° C. After stirring 1 hour, the phases were separated; the organic phase was washed with saturated aqueous NaCl and concentrated to obtain compound R-2.

Step B: A mixture of compound R-2 (1.0 g) and 4-amino tetrahydropyran (1.58 g) was heated at 180° C. under nitrogen for 45 minutes, then cooled to room temperature and concentrated. The resulting residue was diluted with methylene chloride, and sodium hydroxide (1N, 2 mL) was added. The resulting layers were separated and the aqueous layer was extracted with methylene chloride. The combined organic layers were washed with brine, dried over sodium sulfate and concentrated. The resulting residue was purified by crystallization from heptane/ethyl acetate (3:1) to give compound R-3. ESI-MS calc. for C₁₃H₁₇F₂NO₂: 257; Found: 258 (M+H).

Step C: A mixture of compound R-3 (1.5 g) and acrylonitrile (9.6 mL) was heated at 80° C. under nitrogen. After heating 20 hours, ethanol (0.34 mL) and formamide (0.23 mL) were added and heating was continued for another 16 hours. The resulting reaction mixture was concentrated to give a residue; the residue was diluted with ethyl acetate, washed with brine, dried over sodium sulfate and concentrated. The resulting residue was purified by flash column chromatography on silica gel (12-50% ethyl acetate in hexane) to give colorless oil of compound R-4. ESI-MS calc. for C₁₆H₂₀F₂N₂O₂: 310; Found: 311 (M+H).

Step D: To a solution of compound R-4 (1.3 g) in dry THF (6.5 mL) at −20° C. was added diethyl chlorophosphate (0.64 mL). LiHMDS (1.0 M in THF solution; 8.8 mL) was slowly added over 40 minutes and stirred at −15° C. for 2 hrs. The reaction mixture was quenched with water (10.3 mL), extracted with n-heptane, washed with brine, dried over sodium sulfate and concentrated to give a colorless oil of compound R-5. ESI-MS calc. for C₁₆H₁₈F₂N₂O: 292; Found: 293 (M+H).

Step E: To a solution of compound R-5 (1.2 g) in ethanol (6 mL) was added 50% NaOH (0.65 mL). The solution was heated to reflux (90° C.) under nitrogen for 18 hours, then diluted with ethanol (4 mL) and methanol (10 mL), and cooled to 0° C. The pH of the solution was adjusted to pH 6-7 with H₂SO₄ and Na₂SO₄ was added. The mixture was stirred for 10 minutes, filtered, rinsed with methanol/ethanol (1:1), and the filtrate was concentrated to give solid R-6. ESI-MS calc. for C₁₆H₁₉F₂NO₃: 311; Found: 312 (+H).

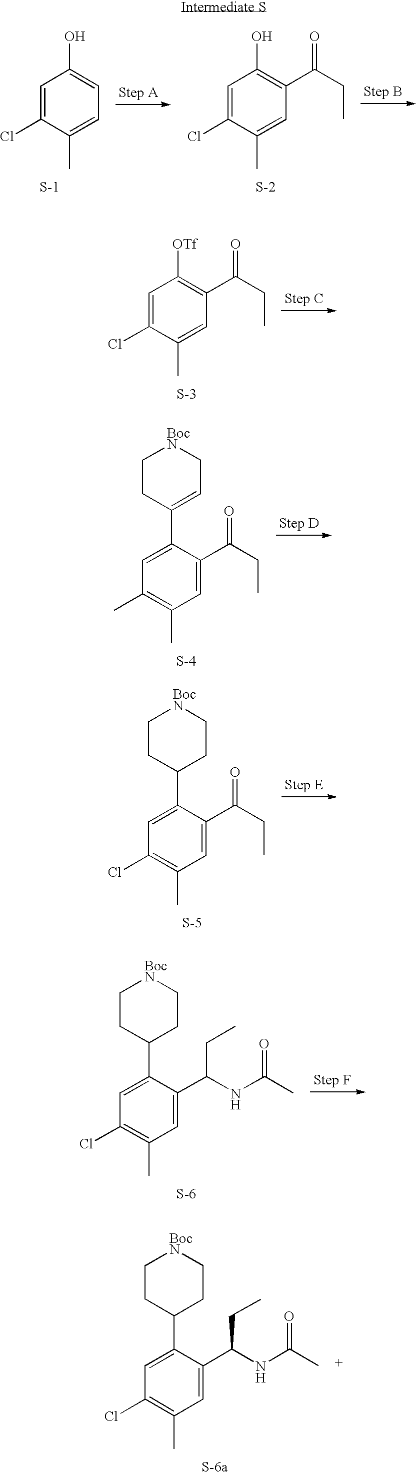

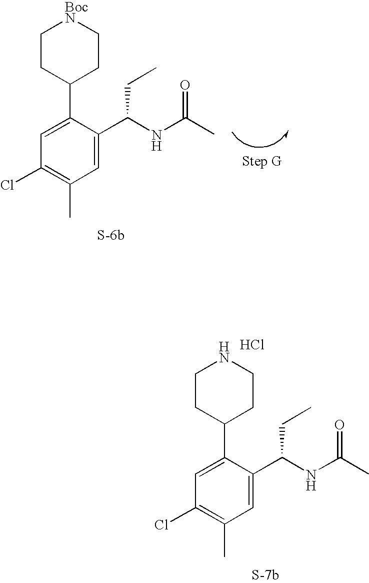

Step A: To slurry of 3-chloro-4-methylphenol S-1 (5.00 g, 35.1 mmol) and propionic chloride (3.35 mL, 38.6 mmol) was added aluminum trichloride (4.68 g, 35.1 mmol) portionwise and gas evolution began. When gas evolution ceased, the reaction was heated up to 180° C. for 1 h and the slurry became a yellow solid. The reaction mixture was cooled to room temperature and treated with a mixture of 25 mL of concentrated HCl aqueous solution and 100 mL of water. The suspension was stirred vigorously for 3 h and the fluffy solid was filtered and washed with cool water. The solid was then placed under high vacuum to dryness to give compound S-2.

Step B: To a solution of 1-(4-chloro-2-hydroxy-5-methylphenyl)propan-1-one S-2 (5.50 g, 27.7 mmol) in methylene chloride (50 mL) was added dimethylamino-pyridine (0.338 g, 2.77 mmol) and the solution was cooled to −78° C. Triethyl amine (4.63 mL, 33.2 mmol) was added, followed by the dropwise addition of trifluoromethane sulfonic anhydride (5.45 mL, 9.14 mmol) over a period of 30 min, keeping the reaction temperature below −70° C. The reaction mixture was stirred at −78° C. for 30 min, then poured into ice-cooled water, and diluted with EtOAc. The resulting layers were separated; the aqueous layer was extracted with 2×200 mL EtOAc. The combined organic layers were washed with brine, dried over anhydrous MgSO₄, and concentrated to give a crude residue. Purification of the crude residue by flash chromatography (silica gel, 10% ethyl acetate/hexanes) afforded compound S-3 as a white wax-like solid.

Step C: To a mixture of 5-chloro-4-methyl-2-propionylphenyl trifluoromethanesulfonate S-3 (9.00 g, 27.2 mmol), absolute ethanol (60 mL), toluene (60 mL), and 2M aqueous sodium carbonate (50 mL) was added tert-butyl 4-(4,4,5,5-tetramethyl-1,3,2-dioxaborolan-2-yl)-3,6-dihydropyridine-1(2H)-carboxylate (8.42 g, 27.2 mmol). The mixture was evacuated and flushed with nitrogen three times. After addition of tetrakis(triphenylphosphine)palladium(0) (3.16 g, 2.72 mmol), the mixture was heated at 75° C. for 1 h. After cooling to room temperature, the mixture was poured into water and extracted with EtOAc (3×250 mL). The combined organic layers were washed with brine, dried over anhydrous MgSO₄, filtered, and concentrated to give a black oil. The crude product was purified via flash column chromatography (silica gel, 10% EtOAc in hexane) to give compound S-4 as colorless oil.

Step D: A solution of tert-butyl 4-(3-chloro-4-methyl-2-propionylphenyl)-piperid-3-ene-1-carboxylate S-4 (6.90 g, 19.0 mmol) in 200 mL of ethanol was added platinum oxide (0.431 mg, 1.90 mmol). After purging with hydrogen three times, the mixture was stirred overnight under hydrogen at atmospheric pressure at room temperature. The resulting solid was filtered and washed with EtOH three times. The filtrates were combined and concentrated to give crude product. The crude product was purified by a flash column chromatography (silica gel, 10% to 20% EtOAc/hexane gradient elution) to give compound S-5 as a white solid.

Step E: To a solution of tert-butyl 4-[5-chloro-4-methyl-2-(1-hydroxy-3-methylbutyl)phenyl]piperidine-1-carboxylate S-5 (2.00 g, 5.44 mmol) in acetonitrile (60 mL) was added concentrated H₂SO₄ (4.35 mL, 81.5 mmol) in 30 mL of acetonitrile. The mixture was stirred at 60° C. overnight. After cooling to room temperature, the mixture was quenched with water and stirred for 30 min, followed by addition of aqueous 5N NaOH until the mixture pH was pH 9. The mixture was extracted with EtOAc (3×150 mL) and the combined organic layers were dried over Na₂SO₄, filtered, and concentrated to give a residue. The residue was dissolved in dichloromethane (20 mL), and Boc₂O (1.78 g, 8.15 mmol) and triethylamine (10.0 mL) were added. The mixture was stirred at room temperature overnight, then concentrated. Purification of the resulting crude residue by flash chromatography (silica gel, gradient elution: 5 to 20% isopropanol/hexanes) afforded compound S-6 as oil.

Step F: The racemic mixture of tert-butyl 4-{2-[1-(acetylamino)propyl]-5-chloro-4-methylphenyl}piperidine-1-carboxylate S-6 (2.00 g, 4.89 mmol) was separated using high performance liquid chromatography over ChiralCel AD 20×250 mm (˜50 mg per injunction; isocratic elution, 3% EtOH in heptane; flow rate=9 mL/min; UV detector wavelength=254) to afford compounds S-6a and S-6b as white solids.

Step G: To a solution of compound S-6b (3.4 g, 8.31 mmole) in CH₂Cl₂ (25 mL) was added HCl (4.0 M in dioxane, 60 mL) and the reaction mixture was stirred at rt for half hour and reaction mixture was concentrated to give the HCl salt S-7b as a white solid. ESI-MS calc. for C₁₇H₃₅ClN₂O: 308, Found: 309 (M+H).

Intermediate T was prepared starting from 3-chloro-4-fluorophenol following procedures similar to that described above for Intermediate S:

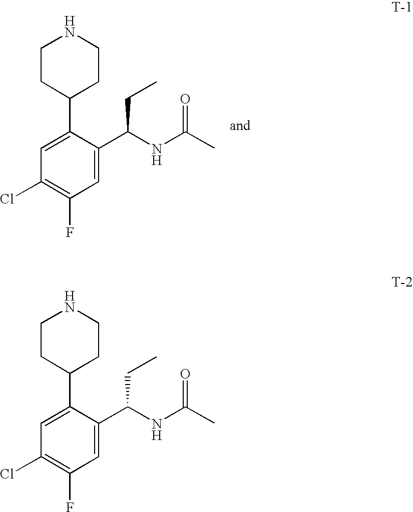

### Example 1

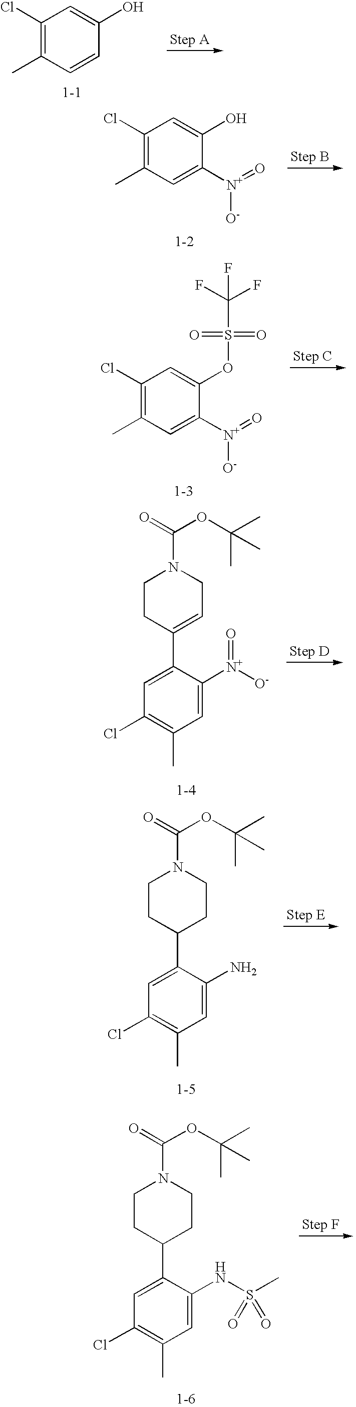

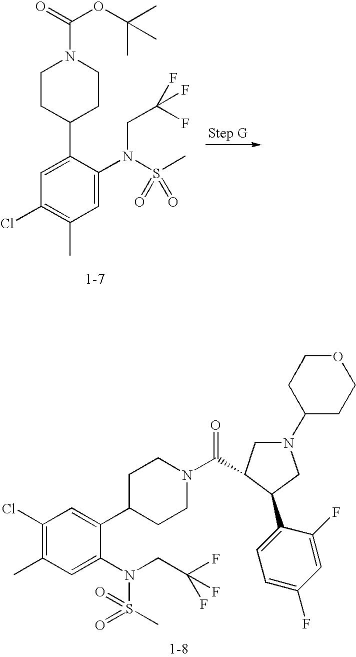

Step A: 5-chloro-4-methyl-2-nitrophenol (1-2) Commercially available 3-chloro-4-methylphenol 1-1 (10.0 g, 70.1 mmol) was dissolved in a solution of ether (280 mL) and dichloromethane (140 mL). To this was added a solution of sodium nitrate (5.97 g, 70.2 mmol) in water (86 mL) and concentrated HCl (56 mL), followed by a catalytic amount of acetic anhydride (0.775 mL, 8.2 mmol). The two phase mixture was stirred vigorously with a magnetic stirrer overnight. The layers were then separated and the organic layer was washed one time each with water and brine, dried over MgSO₄, filtered and the filtrate evaporated in vacuo leaving a red oil. Purification by flash chromatography on silica gel (hexane-ethyl acetate, 9:1) gave 1-2 as a yellow solid. ESI-MS (negative mode) Calculated for C₇H₆ClNO₃: 187; Found: 186 (M−H).

Step B: 5-chloro-4-methyl-2-nitrophenyltrifluoromethanesulfonate (1-3) Nitrophenol 1-2 (2.13 g, 11.8 mmol), 4-dimethylaminopyridine (0.145 g, 1.19 mmol) and triethylamine (1.97 mL, 14.1 mmol) were dissolved in dichloromethane (21 mL) and the clear orange solution was cooled to −78° C. Triflic anhydride (2.36 mL, 14 mmol) was added drop wise over a period of 5 minutes by which time the orange color changed to pale yellow. The reaction mixture was stirred at this temperature for 1.5 h, poured into water and the layers separated. The aqueous layer was extracted two times with dichloromethane and the combined organic extracts washed once with brine, dried over MgSO₄, filtered and the filtrate evaporated to dryness. The yellow oily residue was then dried under high vacuum giving the triflate 1-3, which was used without further purification. ESI-MS Calculated for C₈H₅ClF₃NO₅S: 319; Found: 320 [M+H]⁺.

Step C: tert-butyl 4-(5-chloro-4-methyl-2-nitrophenyl)-3,6-dihydropyridine-1(2H)-carboxylate (1-4) Triflate 1-3 (3.75 g, 11.8 mmol) was mixed with tert-butyl 4-(4,4,5,5-tetramethyl-1,3,2-dioxaborolan-2-yl)-3,6-dihydropyridine-1(2H)-carboxylate (3.64 g, 11.8 mmol) and PdCl₂(dppf) (0.292 g, 0.36 mmol) in DMF (45 mL). A suspension of Na₂CO₃ (6.32 g, 59.6 mmol) in water (13.5 mL) was then added and the mixture was degassed three times by alternate N₂/vacuum purges. The suspension was heated to 90° C. under N₂ with stirring for 1.5 hours, then cooled to rt and stirred overnight. The reaction mixture was diluted with water and extracted with ethyl acetate three times. The combined extracts were washed with brine, dried over MgSO₄, filtered and the filtrate evaporated to dryness. The dark brown oil was purified by flash chromatography on silica gel (hexane-ethyl acetate, 9:1) to give 1-4 as a yellow oil. ESI-MS Calculated for C₁₇H₂₁ClN₂O₄: 352; Found: 375 [M+Na]⁺.

Step D: tert-butyl 4-(2-amino-5-chloro-4-methylphenyl)piperidine-1-carboxylate (1-5) Nitro dihydropyridine 1-4 (2.367 g, 6.7 mmol) was dissolved in ethanol (24 mL) and PtO₂ (0.475 g) was added. Reduction was carried out on a Parr shaker in the presence of H₂ at 45 psi for 7 h 45 min. The catalyst was removed by filtration through a bed of Celite® and the filtrate evaporated. Purification of the crude product by flash chromatography on silica gel (hexane-ethyl acetate, 1:1) gave the aniline 1-5 as an amorphous foam. ESI-MS Calculated for C₁₇H₂₅ClN₂O₂: 324; Found: 347 [M+Na]⁺.

Step E: tert-butyl 4-{5-chloro-4-methyl-2-[(methylsulfonyl)amino]phenyl}piperidine-1-carboxylate (1-6) Aniline 1-5 (0.150 g, 0.46 mmol) and methanesulfonyl chloride (0.072 mL, 0.92 mmol) were dissolved in pyridine (2 mL), the solution heated at 60° C. for 5 h and then cooled to rt and stirred overnight. The pyridine was removed in vacuo, and replaced with ethyl acetate. The mixture was washed one time each with: water, 2N HCl, saturated aqueous NaHCO₃ and brine, then dried over MgSO₄, and filtered. The resulting filtrate was evaporated in vacuo to give 1-6 as an amorphous foam, which was used without further purification.

Step F: tert-butyl 4-{5-chloro-4-methyl-2-[(methylsulfonyl)(2,2,2-trifluoroethyl)-amino]phenyl}piperidine-1-carboxylate (1-7) Sulfonamide 1-6 (0.100 g, 0.25 mmol) was dissolved in DMF (1 mL) and a 60% oil dispersion of NaH (0.022 g, 0.54 mmol) was added. The suspension was heated to 60° C. and stirred at this temperature until all solids reacted (˜5 min). 2,2,2-trifluoroethyl methanesulfonate (0.240 mL, 2.0 mmol) was added, the temperature was raised to 130° C. and the reaction mixture stirred for 72 h. The solution was cooled to rt, diluted with saturated aqueous NH₄Cl—H₂O, and extracted three times with ethyl acetate. The combined organic extracts were washed with brine, dried over MgSO₄, filtered and the filtrate evaporated to dryness. The resulting amber oil was purified by preparative TLC (silica gel, 20×20 cm plate, 1000μ thickness, hexane-ethyl acetate, 3:1) to give 1-7 as an oil. ESI-MS Calculated for C₂₀H₂₈ClF₃N₂O₄S: 484; Found: 485 [M+H]⁺.

Step G: N-[4-chloro-2-(1-{[(3S,4R)-4-(2,4-difluorophenyl)-1-(tetrahydro-2H-pyran-4-yl)pyrrolidin-3-yl]carbonyl}piperidin-4-yl)-5-methylphenyl]-N-(2,2,2-trifluoroethyl)methane-sulfonamide (1-8) 4M HCl in dioxane (3 mL) was added to a solution of BOC protected sulfonamide 1-7 (0.081 g, 0.166 mmol) in dichloromethane (2 mL) and stirred at rt for 1 hour. The solvents were evaporated and the solid residue was stirred with ether (5 mL) and the ether removed with a pipette. The process was repeated and the residue dried briefly under vacuum and then dissolved in dichloromethane (3 mL) with N,N-diisopropylethylamine (0.072 mL, 0.416 mmol). The resulting solution was added to a stirring solution of (3S,4R)-4-(2,4-difluorophenyl)-1-(tetrahydro-2H-pyran-4-yl)pyrrolidine-3-carboxylic acid (0.063 g, 0.2 mmol), HOBt (0.031 g, 0.2 mmol) and EDC (0.048 g, 0.249 mmol) in dichloromethane (3 mL). The reaction mixture was stirred overnight at rt, diluted with dichloromethane and washed one time each with water, saturated aqueous NaHCO₃ and brine, dried over MgSO₄, filtered and evaporated. The crude product was purified by preparative TLC (silica gel, 20×20 cm plate, 1000μ thickness, hexane-ethyl acetate-MeOH, 12:8:2) giving 1-8 as a white solid. ESI-MS Calculated for C₃₁H₃₇ClF₅N₃O₄S: 677; Found: 678 [M+H]⁺.

### Example 2

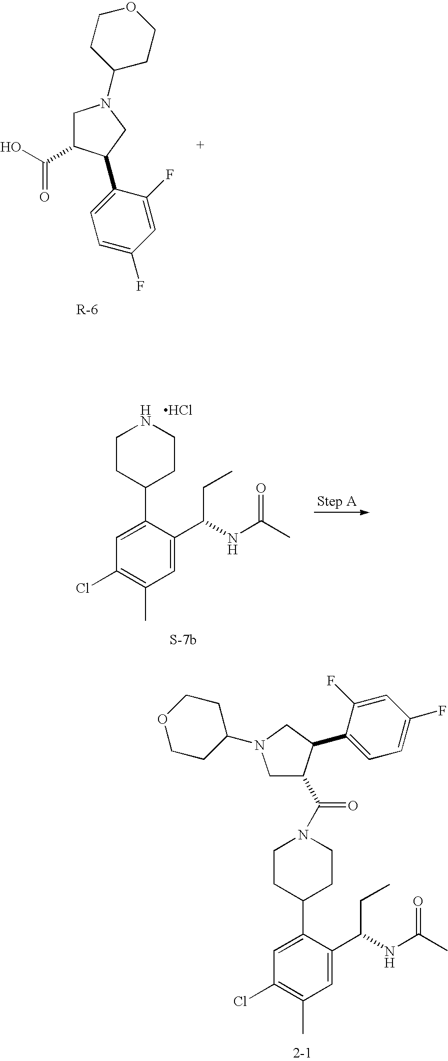

Step A: 5-chloro-4-methyl-2-nitrophenol (1-2) To a solution of Compound R-6 (9.58 mg, 0.0308 mmol) in dichloromethane (3 mL) was added NMM (0.0154 mL, 0.14 mmol), HOBT, (7.6 mg, 0.056 mmol), EDC (10.7 mg, 0.056 mmol) and amine S-7b (9.7 mg, 0.028 mmol). The reaction mixture was stirred at room temperature overnight, diluted with dichloromethane, washed with brine, dried over anhydrous sodium sulfate, filtered, and concentrated. The resulting residue was purified by prep HPLC (20-70% acetonitrile in water) to give compound 2-1 as a white solid. ESI-MS Calculated for C₃₃H₄₂C1F₂N₃O₃: 602; Found: 603 (M−H).

The compounds in Table 1 were prepared using the appropriate reagents, including intermediates T-2 and U-2, and following procedures similar to that described above for Example 2

TABLE 1

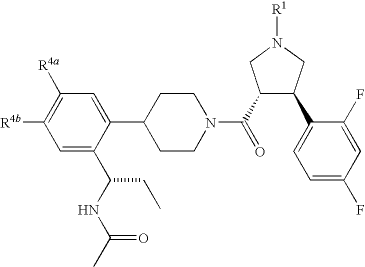

| | | | | Parent Ion m/z |
|---|---|---|---|---|
| Example | R¹ | R^{4a} | R^{4b} | (M + H) |
| 3 | 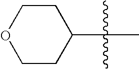 | Cl | F | 606 |
| 4 | 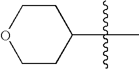 | F | CH₃ | 587 |

### Example 5

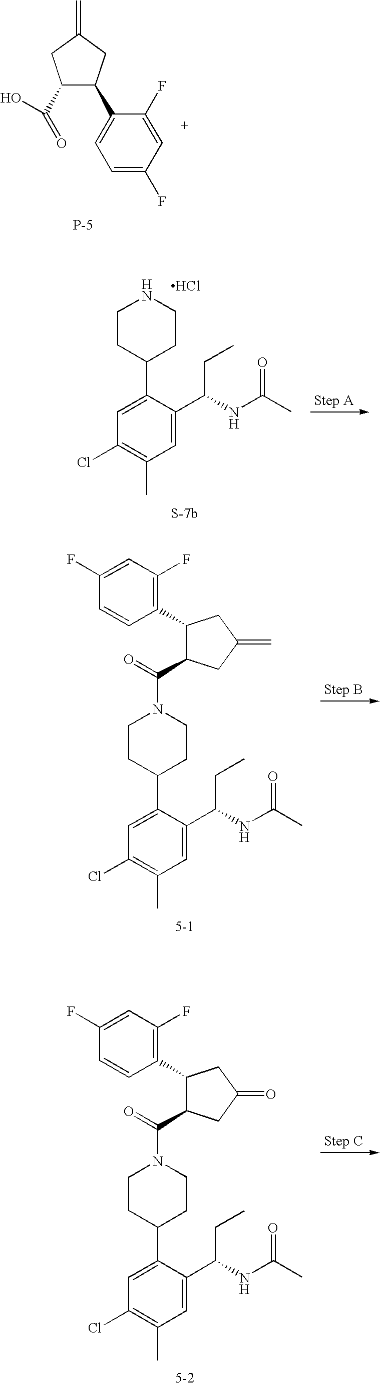

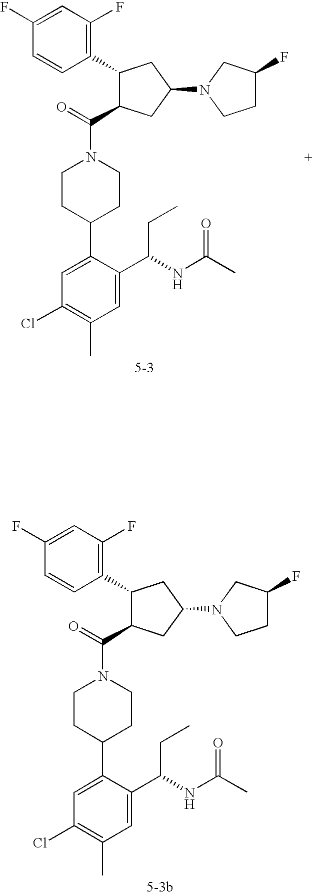

Step A: N-{(1S)-1-[4-chloro-2-(1-{[(1 R,2R)-2-(2,4-difluorophenyl)-4-methylene-cyclopentyl]carbonyl}-piperidin-4-yl)-5-methylphenyl]propyl}acetamide (5-1). To a solution of tert-butyl 4-{2-[(1S or 1R)-1-(acetylamino)propyl]-5-chloro-4-methylphenyl}piperidine-1-carboxylate S-7b (1.00 g, 2.45 mmol) in dichloromethane (1.0 mL) was added 4 N HCl in dioxane (10 mL) and the mixture was stirred at room temperature for 30 min and the reaction was completed. The volatiles were removed under reduced pressure to dryness. The residue was dissolved in dichloromethane (20 mL) and (1R,2R)-2-(2,4-difluorophenyl)-4-methylenecyclopentanecarboxylic acid P-5 (0.641 g, 2.69 mmol), O-(7-azabenzotriazol-1-yl)-N,N,N′,N′,-tetramethyluronium hexafluorophosphat (HATU, 1.12 g, 2.93 mmol), 1-hydroxyl-7-azabenzotriazole (HOAT, 0.399 g, 2.93 mmol), and DIEA (1.24 mL, 7.34 mmol) were added. The mixture was stirred at room temperature overnight and quenched with 1N HCl aqueous solution and extracted with EtOAc three times. The combined organic layer was washed with a saturated NaHCO₃ aqueous solution and brine, dried over MgSO₄, filtered, and concentrated to give compound 5-1 as a white solid. ESI-MS Calculated for C₃₀H₃₅ClF₂N₂O₂: 528; Found: 529 (M+H).

Step B: N-{(1S)-1-[4-chloro-2-(1-{[(1R,2R)-2-(2,4-difluorophenyl)-4-oxocyclopentyl]-carbonyl}piperidin-4-yl)-5-methylphenyl]propyl}acetamide (5-2). To a solution of compound 5-1 (0.500 g, 0.945 mmol) in THF/H₂O (1:1, 20 mL) at RT was added OsO₄ (0.961 mL, 0.095 mmol, 2.5 wt % in tBuOH), followed by a solution of NaIO₄ (0.505 g, 2.363 mmol) in H₂O (3.50 mL) over 30 minutes. The mixture was stirred at room temperature for 1 h before the addition of Na₂SO₃ in H₂O until organic layer was clear. Solid was filtered and rinsed with EtOAc. Organic layer was separated and aqueous layer was extracted with EtOAc. The combined organic layers were washed with brine, dried over anhydrous MgSO₄, filtered, concentrated to give a residue. The residue was purified by flash column chromatography with EtOAc in hexane to give compound 5-2.

Step C: N-((1S)-1-{4-chloro-2-[1-({(1S,2R,4R)-2-(2,4-difluorophenyl)-4-[(3S)-3-fluoropyrrolidin-1-yl]cyclopentyl}carbonyl)piperidin-4-yl]-5-methylphenyl}propyl)acetamide (5-3a) and N-((1S)-1-{4-chloro-2-[1-({®1S,2R,4S)-2-(2,4-difluorophenyl)-4-[(3S)-3-fluoropyrrolidin-1-yl]cyclopentyl}carbonyl)piperidin-4-yl]-5-methylphenyl}propyl)acetamide (5-3b). To a solution of compound 5-2 (0.175 g, 0.330 mmol) in DCM (10.0 mL) was added (S)-3-floropyrrolidine hydrogen chloride (0.124 g, 0.989 mmol), N,N-diisopropylethylamine (0.426 g, 3.30 mmol) and molecular sieves (0.5 g). After the mixture was stirred at room temperature for 30 minutes, a solution of sodium triacetoxyborohydride (0.349 g, 1.65 mmol) was added. The mixture was then stirred at RT overnight. Solid was filtered and washed with DCM. The filtrates were washed with brine, dried over Na₂SO₄, and concentrated. The crude product was purified by prep-TLC with 90:9:1 DCM:MeOH:NH₄OH to give compound 5-3a and 5-3b. The compound 5-3a: ESI-MS Calculated for C₃₃H₄₁ClF₃N₃O₂: 603; Found: 604 (M+H); The compound 5-3b: ESI-MS Calculated for C₃₃H₄₁ClF₃N₃O₂: 603; Found: 604 (M+H).

### Example 6

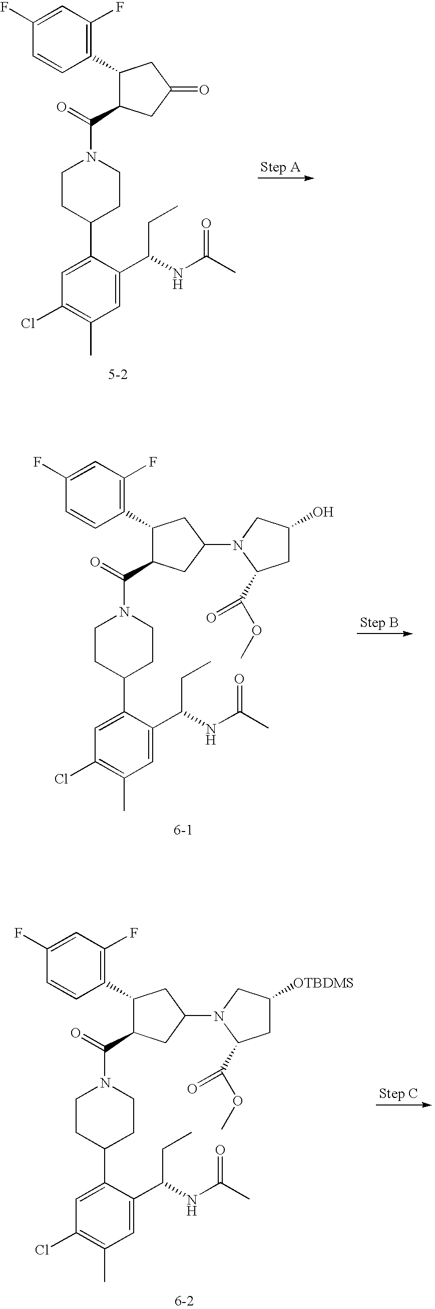

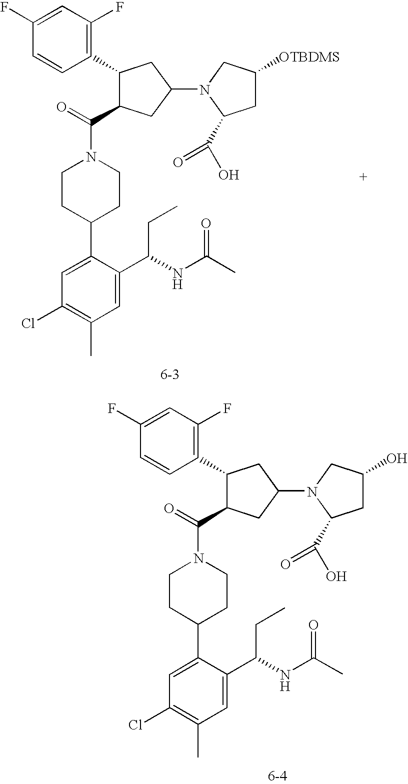

Step A: methyl (4R)-1-[(3R,4R)-3-[(4-{2-[(1S)-1-(acetylamino)propyl]-5-chloro-4-methylphenyl}piperidin-1-yl)carbonyl]-4-(2,4-difluorophenyl)cyclopentyl]-4-hydroxy-D-prolinate trifluoroacetate (6-1). To a solution of compound 5-2 (0.500 g, 0.942 mmol) in DCM (25 mL) was added methyl cis 4-hydroxyl-D-proline (0.273 g, 1.88 mmol), N,N-diisopropylethylamine (0.730 g, 5.65 mmol) and molecular sieves (1.0 g). After the mixture was stirred at room temperature for 30 min, a solution of NaBH(OAc)₃ (1.20 g, 5.65 mmol,) was added. The mixture was stirred at RT overnight. Solid was filtered and washed with DCM. The filtrates were concentrated and purified by HPLC to give compound (6-1) as solid. ESI-MS Calculated for C₃₅H₄₄ClF₂N₃O₅: 659; Found: 660 (M+H).

Step B: methyl (4R)-1-[(3R,4R)-3-[(4-{2-[(1S)-1-(acetylamino)propyl]-5-chloro-4-methylphenyl}piperidin-1-yl)carbonyl]-4-(2,4-difluorophenyl)cyclopentyl]-4-{[tert-butyl(dimethyl)silyl]oxy}-D-prolinate (6-2). To a solution of compound 6-1 (0.500 g, 0.646 mmol) in DCM (10 mL) was added DMAP (0.095 g, 0.775 mmol), N,N-diisopropylethylamine (0.835 g, 6.46 mmol) and TBDMSCl (0.487 g, 3.23 mmol). After the mixture was stirred at room temperature overnight, the mixture was quenched with brine and extracted with ethyl acetate twice. The combined organic layers were washed with brine, dried, and concentrated to give compound (6-2). ESI-MS Calculated for C₄₁H₅₈ClF₂N₃O₅Si: 773; Found: 774 (M+H).

Step C: (4R)-1-[(3R,4R)-3-[(4-{2-[(1S)-1-(acetylamino)propyl]-5-chloro-4-methylphenyl}piperidin-1-yl)carbonyl]-4-(2,4-difluorophenyl)cyclopentyl]-4-{[tert-butyl(dimethyl)silyl]oxy}-D-proline (6-3) and (4R)-1-[(3R,4R)-3-[(4-{2-[(1S)-1-(acetylamino)propyl]-5-chloro-4-methylphenyl}1piperidin-1-yl)carbonyl]-4-(2,4-difluorophenyl)cyclopentyl]-4-hydroxy-D-proline (6-4) To a solution of compound 6-2 (0.498 g, 0.643 mmol) in 5:1 THF:H₂O (2.0 mL) was added LiOH (0.092 g, 3.86 mmol) at 0° C. The mixture was warmed up to room temperature and stirred overnight. The volatiles were removed and the residue was dissolved in MeOH, filtered, and concentrated. The residue was purified by HPLC to give compound 6-3 and compound 6-4. Compound 6-3: ESI-MS Calculated for C₄₀H₅₆ClF₂N₃O₅Si: 759; Found: 760 (M+H); Compound 6-4: ESI-MS Calculated for C₃₄H₄₂ClF₂N₃O₅: 645; Found: 646 (M+H).

The compounds in Table 2 were prepared using the appropriate reagents following procedures similar to that described above for Examples 5 and 6:

TABLE 2

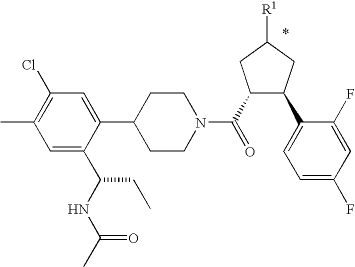

| | | | Parent Ion m/z |
|---|---|---|---|
| Example | R¹ | *chiral center | (M + H) |
| 7 | 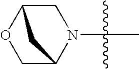 | R and S | 614 |
| 8 | 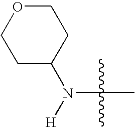 | R and S | 616 |
| 9 | 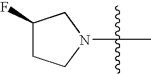 | R | 604 |
| 10 | 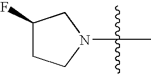 | S | 604 |
| 11 | 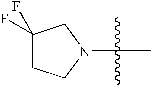 | R | 622 |
| 12 | 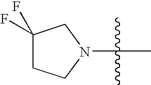 | S | 622 |
| 13 | 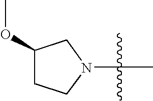 | R | 616 |
| 14 | 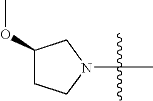 | S | 616 |
| 15 | 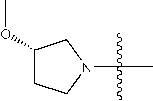 | R | 616 |
| 16 | 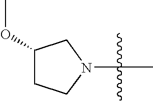 | S | 616 |
| 17 | 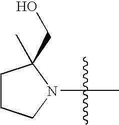 | R and S | 630 |

### Example 18

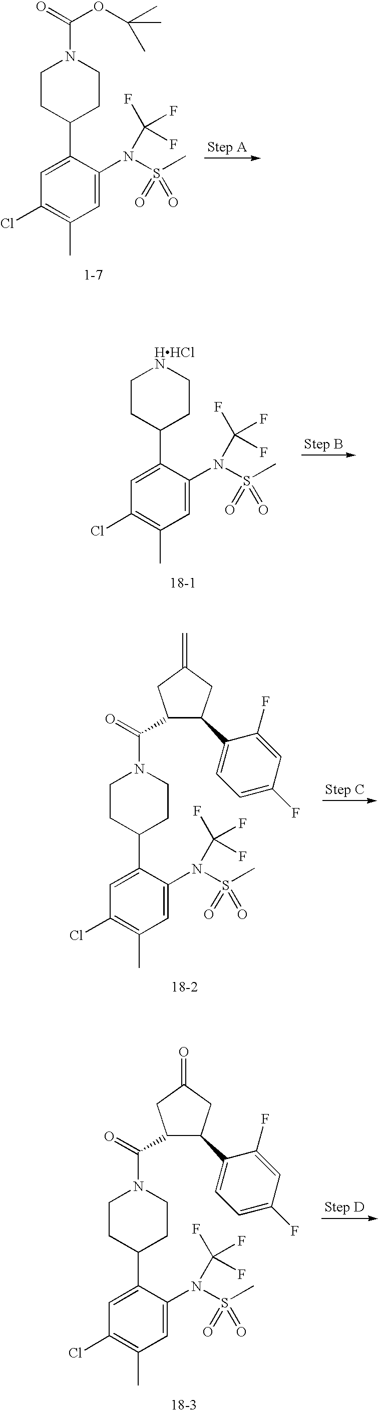

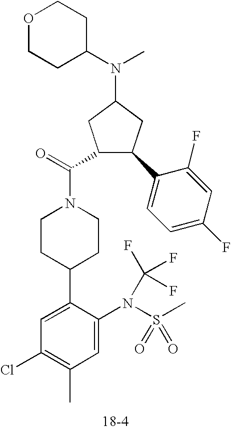

Step A: 4M HCl in dioxane (25 mL) was added to a solution of BOC protected sulfonamide 1-7 (1.01 g, 2.08 mmol) in dichloromethane (5 mL) and stirred at rt for 1 hour. The solvents were evaporated, the solid residue was stirred with ether (15 mL) and the ether was removed with a pipette. The process was repeated and the residue was dried briefly under vacuum to give compound 18-1 as a white solid.

Step B: To a solution of acid P-5 (155.8 mg, 0.654 mmole) in dichloromethane (25 mL) was added N-methylmorpholine (0.098 ml, 0.892 mmole), HOBt (88.3 mg, 0.654 mmole), EDC (170.9 mg, 0.892 mmole) and amine 18-1 (250 mg, 0.5945 mmole). The reaction mixture was stirred at room temperature overnight, diluted with dichloromethane, and washed with water and brine. The organic layer was dried over anhydrous magnesium sulfate, filtered, and concentrated. The resulting residue was purified by MPLC (40S,10-80% EtOAc in hexane) to give 18-2 as yellow oil.

Step C: To a solution of compound 18-2 (0.386 g, 0.639 mmole) in THF (10 ml) and H₂O (10 ml) at room temperature was added OsO₄ (2.5 wt % solution in t-BuOH (0.80 ml, 0.0639 mmole). After stirring the reaction mixture at rt for 10 minutes, sodium periodate (1.92 mmole, 0.410 g in 4 ml H₂O) was added slowly over 15 minutes, and the mixture was stirred for 1.5 hr. Then the solution of sodium thiosulfate pentahydrate (0.476 g, 1.92 mmole, saturated) was added, and the reaction mixture was stirred for an additional 15 minutes. The layers were separated; the aqueous layer was extracted with EtOAc, dried over MgSO₄, filtered and concentrated to give 18-3 as light black solid.

Step D: To a suspension of N-methyltetrahydro-2H-pyrane-4-amine hydrochloride (640 mg, 4.22 mmole) in dichloromethane (12 ml) was added triethylamine (10.55 mmole, 1.47 ml). After stirring at rt for 10 minutes, compound 18-3 (320 mg, 0.5277 mmol), and molecular sieves (4A power, 974 mg) were added. The reaction mixture was stirred at rt for 30 minutes, followed by the addition of sodium triacetoxyborohydride (1.12 g, 5.277 mmol). After stirring 48 hr, the reaction mixture was diluted with CH₂Cl₂, washed with saturated NaHCO₃ and brine, dried over anhydrous sodium sulfate, filtered and concentrated. The resulting residue was purified by prep TLC(CH₂Cl₂: CH₃OH=10:1) to give compound 18-4 as white solid. ESI-MS Calculated for C₃₃H₄₁ClF₅N₃O₄S: 705; Found: 706 [M+H]⁺.

The compounds in Table 3 were prepared using the appropriate reagents following procedures similar to that described above for Example 18:

TABLE 3

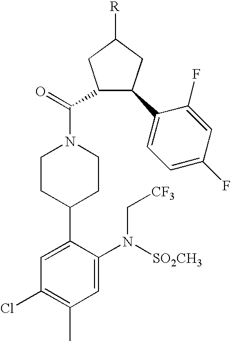

| | | | Parent Ion |
|---|---|---|---|
| | | | m/z |
| | | Calculated | (M + H) |
| Example | R | MW | ESI-MS |
| 19 | 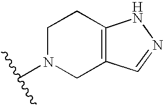 | C₃₃H₃₇ClF₅N₅O₃S 713 | 714 |
| 20 | 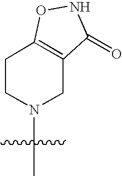 | C₃₃H₃₆ClF₅N₄O₅S 731 | 732 |
| 21 | 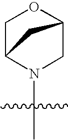 | C₃₂H₃₇ClF₅N₃O₄S 690 | 691 |
| 22 | 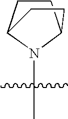 | C₃₃H₃₉ClF₅N₃O₃S 688 | 689 |
| 23 | 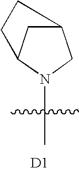 | C₃₃H₃₉ClF₅N₃O₃S 688 | 689 |
| 24 | 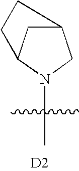 | C₃₃H₃₉ClF₅N₃O₃S 688 | 689 |
| 25 | 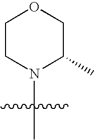 | C₃₂H₃₉ClF₅N₃O₄S 692 | 693 |
| 26 | 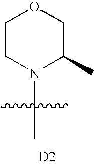 | C₃₂H₃₉ClF₅N₃O₄S 692 | 693 |
| 27 | 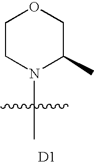 | C₃₂H₃₉ClF₅N₃O₄S 692 | 693 |
| 28 | 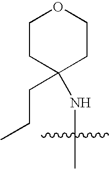 | C₃₅H₄₅ClF₅N₃O₄S 733 | 734 |
| 29 | 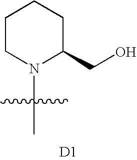 | C₃₃H₄₁ClF₅N₃O₄S 705 | 706 |
| 30 | 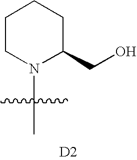 | C₃₃H₄₁ClF₅N₃O₄S 705 | 706 |
| 31 | 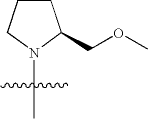 | C₃₃H₄₁ClF₅N₃O₄S 705 | 706 |
| 32 | 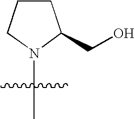 | C₃₂H₃₉ClF₅N₃O₄S 691 | 692 |
| 33 | 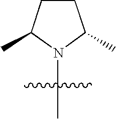 | C₃₃H₄₁ClF₅N₃O₃S 690 | 691 |
| 34 | 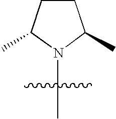 | C₃₃H₄₁ClF₅N₃O₃S 690 | 691 |
| 35 | 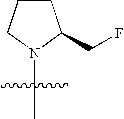 | C₃₂H₃₈ClF₆N₃O₃S 694 | 695 |
| 36 | 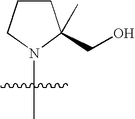 | C₃₃H₄₁ClF₅N₃O₄S 706 | 707 |
| 37 | 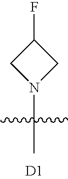 | C₃₀H₃₄ClF₆N₃O₃S 665 | 666 |
| 38 | 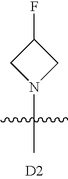 | C₃₀H₃₄ClF₆N₃O₃S 665 | 666 |
| 39 | 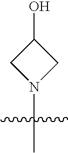 | C₃₀H₃₅ClF₅N₃O₄S 663 | 664 |
| 40 | 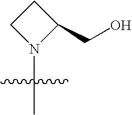 | C₃₁H₃₇ClF₅N₃O₄S 678 | 679 |
| 41 | 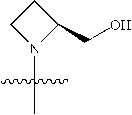 | C₃₁H₃₇ClF₅N₃O₄S 678 | 679 |

### Example 42

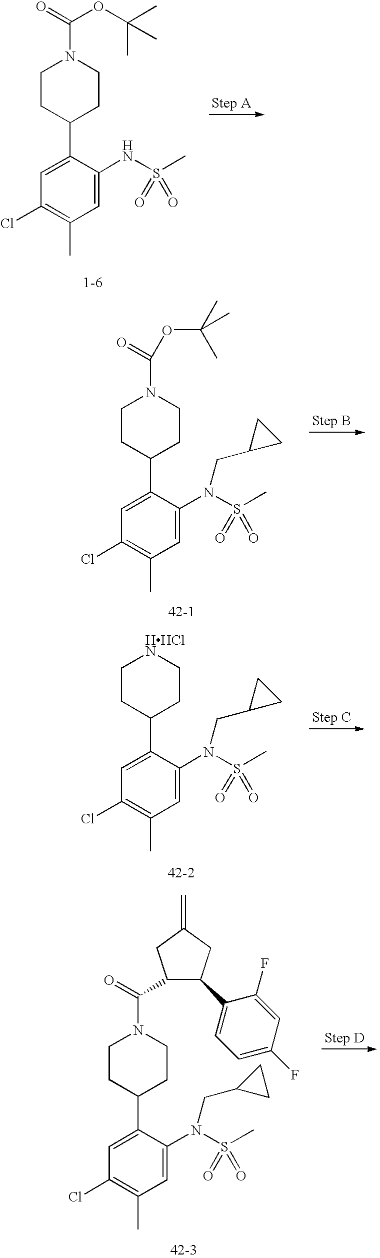

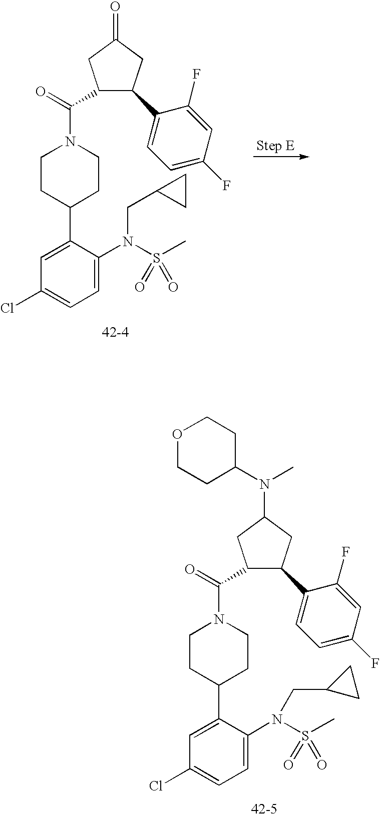

Step A: Sulfonamide 1-6 (1.0 g, 2.48 mmol) was dissolved in DMF (5 ml) and a 60% oil dispersion of NaH (0.199 g, 4.97 mmol) was added. The suspension was heated to 60° C. and stirred at this temperature for 20 minutes, then the reaction mixture was cooled to rt and added bromo methyl cyclopropane. After the reaction mixture was stirred at rt for 18 hours, the reaction mixture was poured into saturated NH₄Cl, extracted with EtOAc, washed with brine, dried over MgSO₄, and concentrated to give a yellow oil which was separated by MPLC (6-50% Etic in hexane) to give compound 42-1 as light yellow oil. ESI-MS Calculated for C₂₂H₃₃ClN₂O₄S: 456; Found: 457 [M+H]⁺.

Step B: Compound 42-2 was prepared from compound 42-1 in an analogous manner to the one described in Step A of Example 18 and using the appropriate reagents. ESI-MS Calculated for C₁₇H₂₅ClN₂O₂S: 356; Found: 357 [M+H]⁺.

Step C: Compound 42-3 was prepared from compound 42-2 in an analogous manner to the one described in Step B of Example 18 and using the appropriate reagents. ESI-MS Calculated for C₃₀H₃₅ClF₂N₂O₃S: 576; Found: 577 [M+H]⁺.

Step D: Compound 42-4 was prepared from compound 42-3 in an analogous manner to the one described in Step C of Example 18 and using the appropriate reagents. ESI-MS Calculated for C₂₉H₃₃ClF₂N₂O₄S: 578; Found: 579 [M+H]⁺.

Step E: Compound 42-5 was prepared from compound 42-4 in an analogous manner to the one described in Step D of Example 18 and using the appropriate reagents. ESI-MS Calculated for C₃₅H₄₆ClF₂N₃O₄S: 677; Found: 678 [M+H]⁺.

The compounds in Table 4 were prepared using the appropriate reagents following procedures similar to that described above for Example 42:

TABLE 4

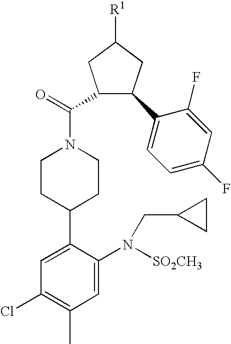

| | | | Parent Ion |
|---|---|---|---|
| | | Calculated | m/z (M + H) |
| Example | R¹ | MW | ESI-MS |
| 43 | 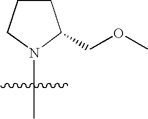 | C₃₅H₄₆ClF₂N₃O₄S 677 | 678 |
| 44 | 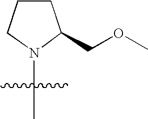 | C₃₅H₄₆ClF₂N₃O₄S 677 | 678 |

### Example 45

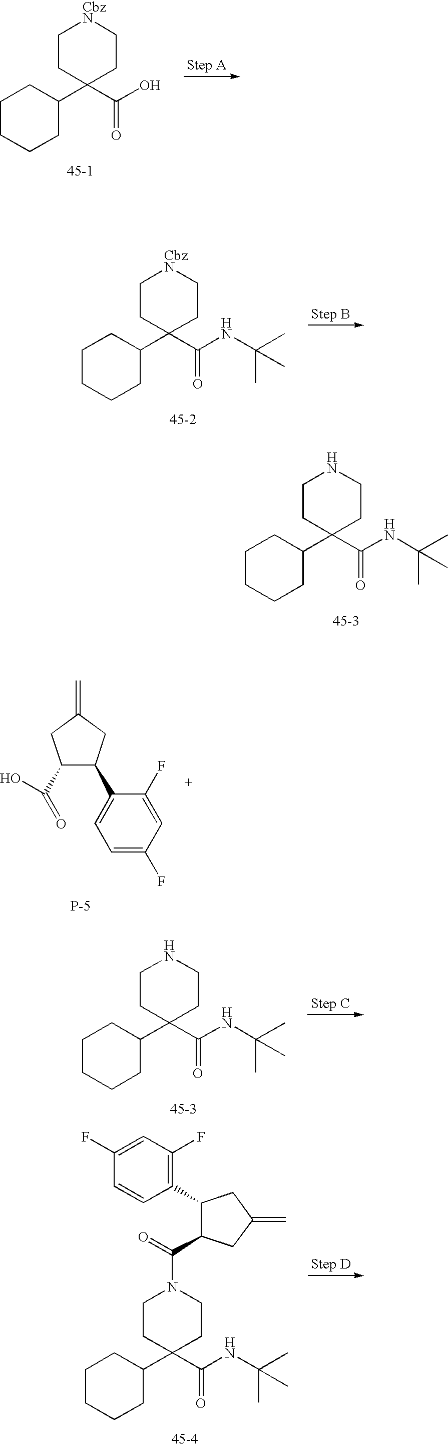

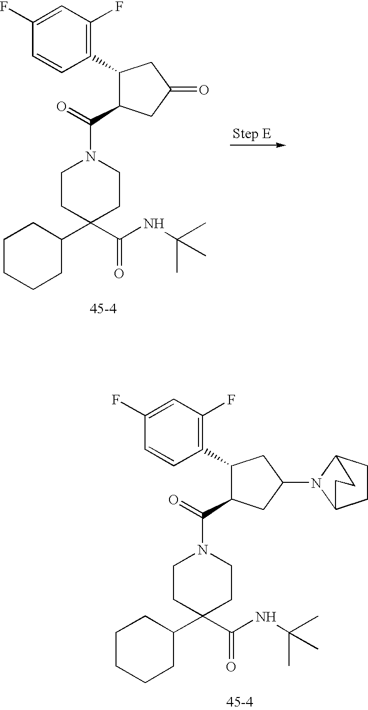

Step A: benzyl 4-[(tert-butylamino)carbonyl]-4-cyclohexylpiperidine-1-carboxylate (45-2). N-(Benzyloxycarbonyl)-4-cyclohexyl-piperidine-4-carboxylic acid (45-1) (2.5 g, 7.24 mmol) was dissolved in 36 mL of CH₂Cl₂ and cooled at 0° C. in an ice-H₂O bath. Oxalyl chloride (2.0 M solution in CH₂Cl₂, 3.98 mL, 7.96 mmol) was then added dropwise followed by the addition of 1-2 drops of DMF. This mixture was stirred at 0° C. for 2 h and then concentrated with toluene. The residue was dissolved in CH₂Cl₂ and cooled at 0° C. in an ice-H₂O bath, and then t-butylamine (2.28 mL, 21.72 mmol) was added dropwise. The reaction mixture was then stirred at 0° C. for 2 h, warmed to room temperature, and stirred at room temperature overnight. The resulting mixture was then diluted with CH₂Cl₂ and washed with brine, dried over MgSO₄, filtered, and concentrated to give 45-2 as a solid. Mass spectrum: Calcd for C₂₄H₃₆N₂O₃: 400.27; Found: 401 (M⁺+1).

Step B: N-(tert-butyl)-4-cyclohexylpiperidine-4-carboxamide (45-3). Compound 45-2 (7.24 mmol) was dissolved in 30 mL of CH₂Cl₂ and then 30% HBr in acetic acid (7.2 mL, 36.15 mmol) was added. The mixture was stirred at room temperature for 45 minutes, then diethyl ether was added. The resulting precipitate was filtered and washed with ether. The solid was dissolved in ethyl acetate and washed with 1N NaOH solution, and the aqueous layer was extracted with EtOAc. The combined organic phases were dried over K₂CO₃, filtered, and concentrated to give 45-3 as a white solid. Mass spectrum: Calcd for C_{≠}H₃₀N₂O: 266.24; Found: 267 (M⁺+1).

Step C: N-(tert-butyl)-4-cyclohexyl-1-{[(1R,2R)-2-(2,4-difluorophenyl)-4-methylenecyclopentyl]carbonyl}piperidine-4-carboxamide (45-4). To a solution of N-(tert-butyl)-4-cyclohexylpiperidine-4-carboxamide 45-3 (1.00 g, 3.75 mmol) in dichloromethane (50 mL) and (1R,2R)-2-(2,4-difluorophenyl)-4-methylenecyclopentanecarboxylic acid P-5 (0.894 g, 3.75 mmol), EDC (0.863 g, 4.50 mmol), HOBt (0.609 g, 4.50 mmol), and DIEA (1.46 g, 11.3 mmol) were added. The mixture was stirred at room temperature overnight and quenched with 1N HCl aqueous solution and extracted with EtOAc three times. The combined organic layer was washed with a saturated NaHCO₃ aqueous solution and brine, dried over MgSO₄, filtered, concentrated and purified by a flash column chromatography on silica gel with 0 to 10% EtOAc in DCM to give compound 45-4. ESI-MS Calculated for C₂₉H₄₀F₂N₂O₂: 486; Found: 487 (M+H).

Step D: N-(tert-butyl)-4-cyclohexyl-1-{[(1R,2R)-2-(2,4-difluorophenyl)-4-oxocyclopentyl]carbonyl}piperidine-4-carboxamide (45-5). To a solution of compound 45-4 (1.00 g, 2.05 mmol) in THF/H₂O (1:1, 40 mL) at rt was added OsO₄ (2.09 mL, 0.21 mmol, 2.5 wt % in tBuOH), followed by a solution of NaIO₄ (1.10 g, 5.14 mmol) in H₂O (7.0 mL) over 30 minutes. The mixture was stirred at room temperature for 1 h before the addition of Na₂SO₃ in H₂O until organic layer was clear. Solid was filtered and rinsed with EtOAc. Organic layer was separated and aqueous layer was extracted with EtOAc. The combined organic layers were washed with brine, dried over anhydrous MgSO₄, filtered, concentrated to give compound 45-5. ESI-MS Calculated for C₂₈H₃₈F₂N₂O₃: 488; Found: 489 (M+H).

Step E: 1-{[(1R,2R)-4-(7-azabicyclo[2.2.1]hept-7-yl)-2-(2,4-difluorophenyl)-cyclopentyl]carbonyl}-N-(tert-butyl)-4-cyclohexylpiperidine-4-carboxamide (45-6). To a solution of compound 45-5 (0.100 g, 0.21 mmol) in DCM (5.0 mL) was added 7-azabicyclo[2.2.1]heptane (0.199 g, 2.05 mmol), N,N-diisopropylethylamine (0.426 g, 3.30 mmol) and molecular sieves (0.5 g). After the mixture was stirred at room temperature for 30 minutes, a solution of sodium triacetoxyborohydride (0.435 g, 2.05 mmol) was added. The mixture was then stirred at RT overnight. Solid was filtered and washed with DCM. The filtrates were washed with brine, dried over Na₂SO₄, and concentrated. The crude product was purified by HPLC to give compound 45-6. ESI-MS Calculated for C₃₄H₄₉F₂N₃O₂: 569; Found: 570 (M+H).

The compounds in Table 5 were prepared using the appropriate reagents following procedures similar to that described above for Example 45:

TABLE 5

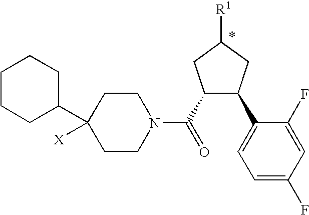

| | | | | Parent Ion m/z |
|---|---|---|---|---|
| Example | R¹ | X | *chiral center | (M + H) |
| 46 | 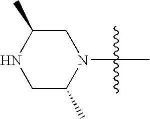 | 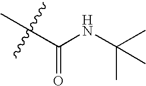 | R and S | 562 |
| 47 | 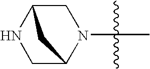 | 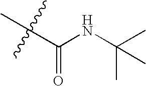 | R and S | 587 |
| 48 | 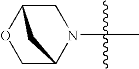 | 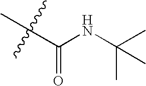 | R and S | 571 |
| 49 | 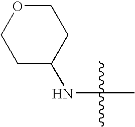 | 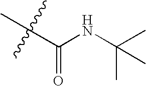 | R and S | 570 |

### Example 50

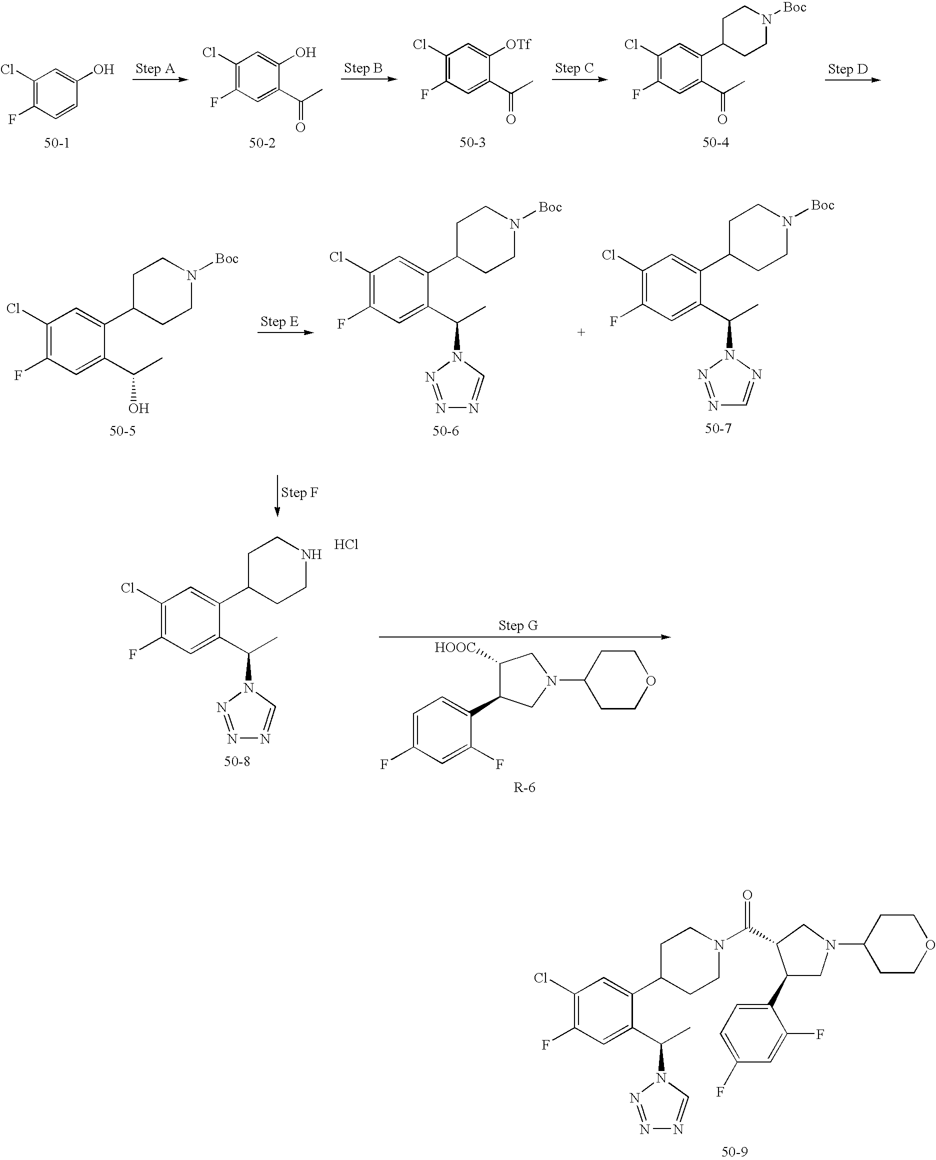

Step A: 1-(4-chloro-5-fluoro-2-hydroxyphenyl)ethanone (50-2) A 500 mL one necked round bottomed flask equipped with condenser was charged 3-chloro-4-fluorophenol 50-1 (10.72 g, 73.14 mmol), aluminum chloride (14.63 g, 109.72 mmol) and acetyl chloride 8.613 g, 109.72 mmol). The mixture was heated slowly to 150° C. over 30 minutes and then at 150° C. for 3 hours. The reaction mixture was cooled to room temperature and diluted with methylene chloride (200 mL) and quenched with HCl (2N, 100 mL). The organic layer was separated and the aqueous layer was extracted with ethyl acetate (3×100 mL). The combined organic phases were washed with water, brine, dire over MgSO₄, filtered and concentrated to afford solid brown colored product 50-2 (Rf=0.4 in ethyl acetate:hexanes=1:4), which was used directly to next step without further purification.

Step B: 2-acetyl-5-chloro-4-fluorophenyl trifluoromethane sulfonate (50-3) A 500 mL one necked round bottomed flask was charged with compound 50-2 (13.70 g, 72.64 mmol), methylene chloride (150 mL), DMAP (0.887 g, 7.26 mmol) and triethyl amine 8.82 g, 87.17 mmol). The mixture was cooled to −78° C. in a dry ice-acetone bath. Then trifluoromethanesulfonic anhydride (23.98 g, 84.99 mmol) was added by syringe dropwise over 30 min. The resulting reaction mixture was stirred at −78° C. for an additional 30 minutes. The reaction mixture was then poured into ice water (200 mL). The organic layer was separated and the aqueous layer was extracted with ethyl acetate (3×200 mL). The combined organic phases were washed with brine, dried over MgSO₄, filtered and concentrated. The residue was purified by MPLC (0 to 10% ethyl acetate in hexanes) to afford product 50-3 as a dark colored sticky oil. (Rf=0.3 by 10% ethyl acetate in hexanes)

Step C: t-butyl 4-(2-acetyl-5-chloro-4-fluorophenyl)piperidine-1-carboxylate (50-4) A 50 mL one necked round bottom flask was charged with compound 50-3 (1.20 g, 3.74 mmol), PdCl₂(dppf) (0.082 g, 0.112 mmol), Cu(I)I (0.043 g, 0.225 mmol), and DMA (6 mL). The flask was sealed by rubber septum. The resulting mixture was degassed with alternating vacuum/nitrogen purges. The filtered piperidylzinc iodide solution (2.82 g, 7.48 mmol, prepared according to J. Org. Chem. 2004, 69, 5120) was then added dropwise via syringe over 5 minutes. The mixture was degassed one more time and then heated to 80° C. for 2 hours. The reaction mixture was cooled to room temperature and poured into ice water (50 mL) and extracted with ether (4×50 mL). The combined organic phases were washed with water (2×), brine, dried over MgSO₄, filtered and concentrated to give a residue. The residue was purified by MPLC (0 to 20% ethyl acetate in hexanes) to afford product 50-4 ((m/z (ES) (M+H)⁺=356, Rf=0.3 by 20% ethyl acetate in hexanes) ¹H NMR (CDCl₃, δ ppm): 7.39 (1H, s), 7.37 (1H, s), 4.24 (2H, br), 3.28 (1H, m), 2.79 (2H, br), 2.58 (3H, s), 1.78 (2H, d, J=13 Hz), 1.54 (2H, m), 1.48 (9H, s).

Step D: t-butyl 4-{5-chloro-4-fluoro-2-[(1 S)-hydroxyl-ethyl]phenyl}piperidine-1-carboxylate (50-5) To a solution of borane diethylaniline complex and (R)-Me-CBS in MTBE (5 mL) in a 100 mL one necked round bottomed flask, was added dropwise a solution of compound 50-4 (1.79 g, 5.03 mmol) in MTBE (9 mL) over 20 min at 40° C. The resulting reaction mixture was then stirred at 40° C. for 2 hours and then allowed to age at room temperature for 2 hours. The reaction was then quenched by adding 10 mL of methanol slowly. After stirring for 10 minutes, HCl (2.0 M, 8 mL) was added over 5 minutes and the solution was stirred further for 10 minutes. The organic layer was separated and the aqueous layer was extracted with ether (3×8 mL). The combined organic phases were washed with water, brine, dried over MgSO₄, filtered and concentrated to give a residuen. The residue was purified by MPLC (0 to 40% ethyl acetate in hexanes) to afford product 50-5 (>99% ee, Rt=11.62 min on Chiral OD column by 5% ethanol in heptane, Rf=0.4 with ethyl acetate:hexanes=2:3, m/z (ES) (M+H)⁺=358). ¹HNMR (CDCl₃, δ ppm): 7.34 (1H, d, J=10.5 Hz), 7.18 (1H, d, J=7.5 Hz), 5.14 (1H, q, J=6 Hz), 4.20 (2H, br), 3.29 (1H, br), 2.88 (1H, m), 2.76 (2H, br), 1.73 (2H, d, J=13 Hz), 1.61 (5H, m), 1.46 (9H, s).

Step E: t-butyl 4-{5-chloro-4-fluoro-2-[(1R)-1-(1H-tetrazole-1-yl)]phenyl}-piperidine-1-carboxylate (A-6) and t-butyl 4-{5-chloro-4-fluoro-2-[(1R)-1-(2H-tetrazole-2-yl)]phenyl}piperidine-1-carboxylate (50-7)

Compound 50-5 (0.346 g, 0.967 mmol) was added to a 250 mL one necked round bottomed flask along with polymer-supported triphenyl phosphine (0.507 g, 1.93 mmol), tetrazole (0.135 g, 1.93 mmol) and methylene chloride (20 mL). The mixture was cooled to 0° C. in ice water bath and then DIAD (0.453 g, 1.93 mmol) was added dropwise through a syringe with stirring. The resulting reaction mixture was stirred at 0° C. for 1 hour and then at room temperature for 1 hour. After the polymer was filtered and washed with methylene chloride. The filtrate was concentrated to give a residue, which was purified by MPLC (0 to 50% ethyl acetate in hexanes) to afford two products: first elute 50-7 (Rf=0.2 with ethyl acetate:hexanes=1:4, 82.4% ee, rt=19.7 min in 7% IPA in heptane on ChiralCel OD column, m/z (ES) (M+H)⁺=410) and second elute 50-6 (29%, Rf=0.2 with ethyl acetate:hexanes=2:3, 95.1% ee, rt=17.6 min by 13% ethanol in heptane on Chiral Pak OD-H column, m/z (ES) (M+H)⁺=410). The regioisomer structure was confirmed by NoeDiff NMR.

Step F: t-butyl 4-{5-chloro-4-fluoro-2-[(1R)-1-(1H-tetrazole-1-yl)]phenyl}piperidine (50-8)

Compound 50-6 (0.023 g, 0.056 mmol) was charged to a 20 mL vial along with methanol (1.5 mL) and HCl (concentrated, 0.5 mL). The resulting mixture was stirred and heated in an oil bath of 40 C for 20 minutes and then concentrated by rotary evaporation to give compound 50-8 as the HCl salt (m/z (ES) (M+H)⁺=310).

Step G: 4-{5-chloro-4-fluoro-2-[(1R)-1-(1H-tetrazol-1-yl)ethyl]phenyl}-1-{[(3S,4R)-4-(2,4-difluorophenyl)-1-(tetrahydro-2H-pyran-4-yl)pyrrolidin-3-yl]carbonyl}-piperidine (50-9)

The amine HCl salt 50-8 (0.056 mmol) was charged in a 20 mL round bottomed flask along with acid R-6, methylene chloride (1 mL) and Hunig's base (0.036 g, 0.28 mmol). The mixture was stirred until the solid dissolved. Then HATU (0.026 g, 0.067 mmol) and HOAT (0.008 g, 0.056 mmol) were added and the resulting reaction mixture was stirred at room temperature for 2 hours. Then the reaction mixture was concentrated to give a residue. The residue was dissolved in methanol (1 mL), filtered through a syringe filtered and washed with methanol (2 mL). The filtrate was concentrated to give a residue, which was purified by reverse phase HPLC(YMC column, 20% to 80% acetonitrile in water) to afford product 50-9 as the TFA salt (m/z (ES) (M+H)⁺=603).

The compounds in Table 6 were prepared using the appropriate reagents following procedures similar to that described above for Example 50 and using the appropriate starting materials:

TABLE 6

| | | Parent Ion |
|---|---|---|
| Example | R¹ | m/z (M + H) |
| 51 | 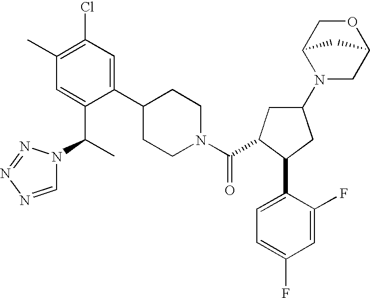 | 611 |
| 52 | 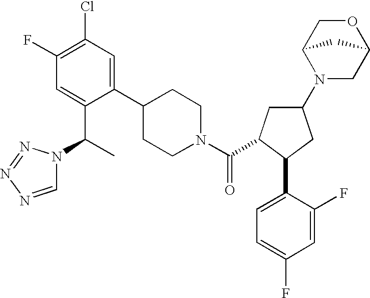 | 615 |
| 53 | 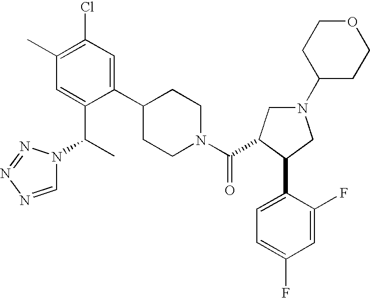 | 599 |
| 54 | 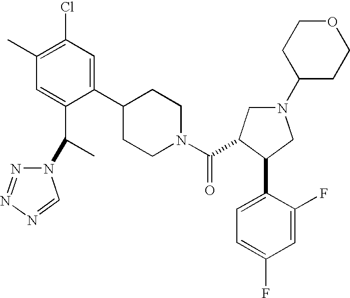 | 599 |
| 55 | 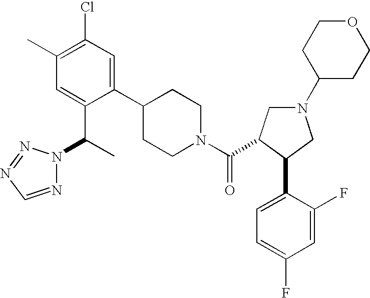 | 599 |

### Example 56

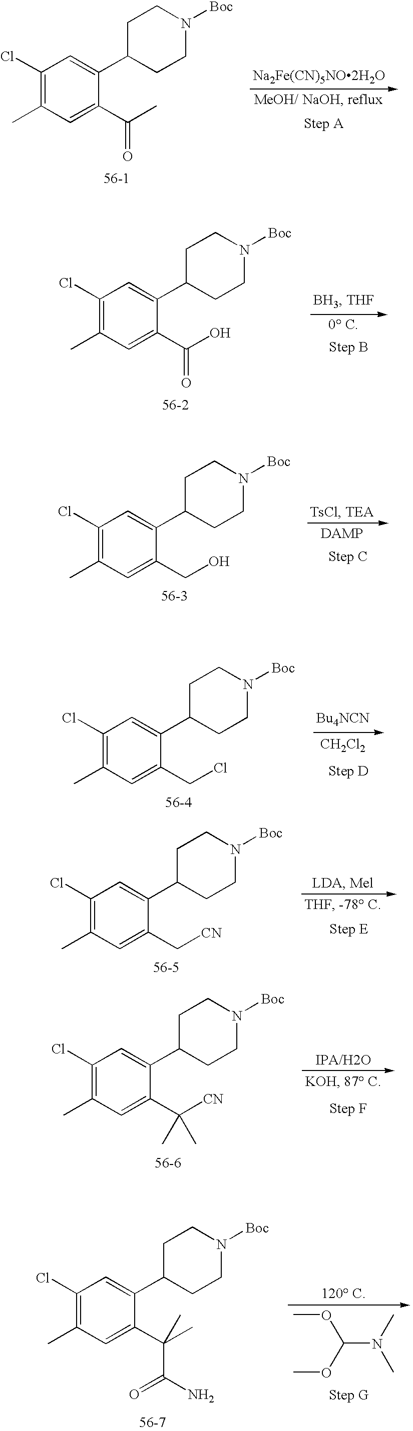

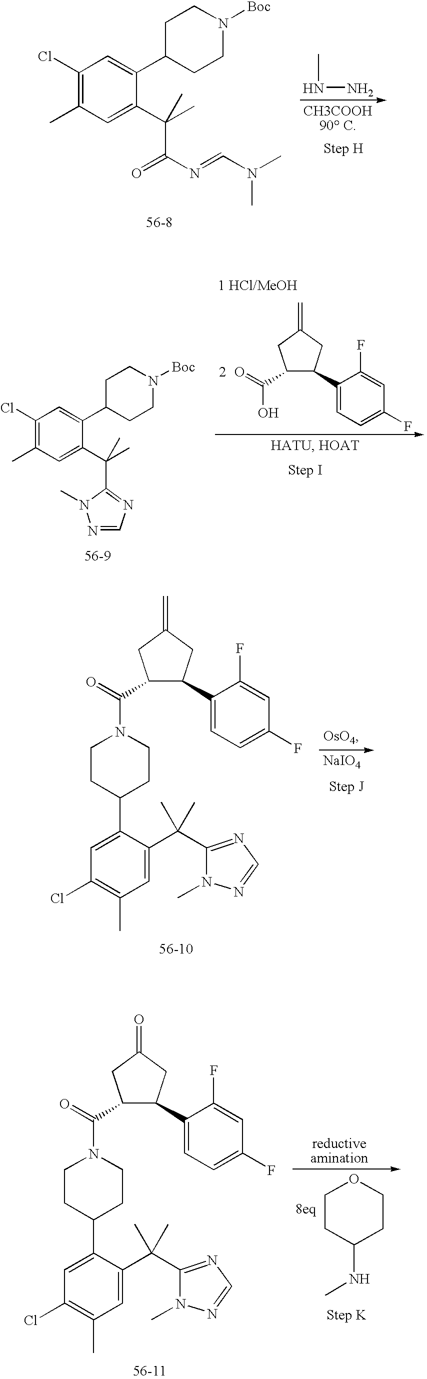

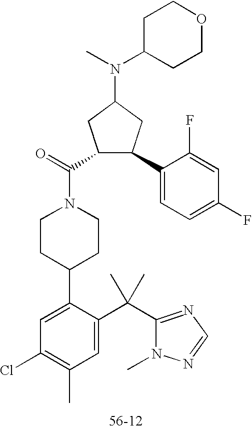

Step A: 2-[1-(t-butoxycarbonyl)piperidin-4-yl]-4-chloro-5-methylbenzoic acid (56-2) To a 100 mL one necked round bottomed flask was charged compound 56-1 (1.8 g, 5.1 mmol, prepared according to the synthesis of Intermediate S) along with methanol/5N NaOH (20/10 mL) and sodium nitroferricyanide (III) dehydrate (3.05 g, 10.2 mmol). The mixture was heated to 80° C. in for 4 hours, then cooled to room temperature, then diluted with ethyl acetate/water (50/50 mL), and neutralized with concentrated HCl to pH=2. The organic layer was separated and the aqueous layer was extracted with ethyl acetate (3×100 mL). The combined organic phases were washed with water, brine, dried over MgSO₄, filtered and concentrated to give a residue. The residue was purified by MPLC (0 to 30% ethyl acetate in hexanes) to afford product 56-2 (m/z (ES) (M+H)⁺=354).

Step B: t-butyl 4-[5-chloro-2-(hydroxymethyl)-4-methylphenyl]piperidine-1-carboxylate (56-3)

To a 100 mL one necked round bottomed flask was charged compound 56-2 (0.40 g, 1.13 mmol) and anhydrous THF (3 mL). The mixture was cooled to 0° C. in an ice bath, then boron hydride in THF (1.5 M, 4 mL) was added by syringe dropwise over 10 minutes. The resulting reaction mixture was stirred at 0° C. for an additional 30 minutes and slowly warmed to room temperature overnight. The reaction mixture was quenched with methanol (5 mL) and stirred for 30 minutes. Then the organic solvent was concentrated to give a residue, which was purified by preparative TLC (40% ethyl acetate in hexanes) to afford product 56-3. (Rf=0.3 by 40% ethyl acetate in hexanes).

Step C: t-butyl 4-[5-chloro-2-(chloromethyl)-4-methylphenyl]piperidine-1-carboxylate (56-4)

To a 50 mL one necked round bottom flask were charged with compound 56-3 (0.21 g, 0.62 mmol), methylene chloride (2 mL), DMAP (catalytic amount) and triethyl amine (188 mg, 1.86 mmol) Then tosyl chloride (177 mg, 0.93 mmol) was added to the mixture, and the mixture was stirred at room temperature for 2 hours. The reaction mixture was concentrated to give a residue, which was purified by preparative TLC (10% ethyl acetate in hexanes) to afford product 56-4 (Rf=0.3 by 10% ethyl acetate in hexanes) ¹HNMR (CDCl₃, δ ppm): 7.21 (1H, s), 6.85 (1H, s), 4.36 (2H, br), 2.81 (2H, br), 2.38 (3H, s), 2.12 (2H, s), 1.78 (2H, d), 1.61 (2H, m), 1.48 (9H, s).

Step D: t-butyl 4-[5-chloro-2-(cyanomethyl)-4-methylphenyl]piperidine-1-carboxylate (56-5)

To a 25 mL one necked round bottom flask were charged with compound 56-4 (0.15 g, 0.42 mmol), and methylene chloride (2 mL). Then Bu₄NCN (0.63 mmol) was added to the mixture, and the mixture was then stirred at room temperature for 2 hours. The reaction mixture was concentrated by rotary evaporation to give a residue, which was purified by preparative TLC (25% ethyl acetate in hexanes) to afford product 56-5 (Rf=0.1 by 10% ethyl acetate in hexanes; m/z (ES) (M+Na)⁺=371).

Step E: t-butyl 4-[5-chloro-2-(1-cyano-1-methylethyl)-4-methylphenyl]piperidine-1-carboxylate (60-6) To a 50 mL one necked round bottomed flask was charged with compound 56-5 (0.170 g, 0.49 mmol) and anhydrous THF (2 mL). The mixture was cooled to −78° C. in a dry ice-acetone bath, then LDA (2 M in THF) was added dropwise by syringe at −78° C. over 10 minutes. The resulting reaction mixture was stirred at −78° C. for an additional 30 minutes. Then methyl iodide (0.207 g, 1.46 mmol) was added dropwise by syringe at −78° C. The resulting reaction mixture was stirred at −78° C. for 2 hours, and then slowly warmed to room temperature overnight. The reaction mixture was then quenched with saturated NH₄Cl (5 mL), stirred for 30 minutes, and extracted with ethyl acetate (3×50 mL). The combined organic phases were washed with water, brine, dried over MgSO₄, filtered and concentrated to give a residue, which was purified by preparative TLC (25% ethyl acetate in hexanes) to afford 56-6 (Rf=0.3 with ethyl acetate:hexane=1:4, m/z (ES) (M+Na)⁺=399).

Step F: t-butyl 4-[2-(2-amino-1,1-dimethyl-2-oxoethyl)-5-chloro-4-methylphenyl]piperidine-1-carboxylate (56-7) Compound 56-6 (0.193 g, 0.51 mmol), potassium hydroxide (0.864 g, 15.4 mmol), isopropanol (10 mL), and water (0.5 mL) in a sealed vessel were heated in an oil bath of 85° C. for 15 hours. The mixture was then cooled to 0° C. in an ice water bath and stirred for 30 minutes. The resulting solid was filtered and washed with cold isopropanol (2 mL) and water (2 mL). The filtrate was concentrated to the water volume, and the solid was filtered and washed with water (2×2 mL). The two crops of solid was combined to afford product 56-7 (m/z (ES) (M+Na)⁺=417).

Step G: t-butyl 4-[5-chloro-2-(2-{[(1E)-(dimethylamino) methylene]amino}-1,1-dimethyl-2-oxoethyl)-4-methylphenyl]piperidine-1-carboxylate (56-8) Compound 56-7 (0.20 g, 0.0.51 mmol) and N,N-dimethylformamide dimethyl acetal was heated in an oil bath of 120° C. for 1 hour. After cooling to room temperature, the excess DMF-DMA was removed by rotary evaporation and the resulting residue was dried by co-evaporation with toluene three times to afford sticky oil product 56-8 (m/z (ES) (M+Na)⁺=450), which was used in next step without further purification.

Step H: t-butyl 4-{5-chloro-4-methyl-2-[1-methyl-1-(1-methyl-1H-1,2,4-triazol-5-yl)ethyl]phenyl}piperidine-1-carboxylate (56-9) A mixture of compound 56-8 and HOAc (2 mL) was cooled to 0° C., and methyl hydrazine (0.046 g, 1.2 mmol) was added dropwise under vigorous stirring. The resulting mixture was heated to 95° C. for 1 hour. After cooling to room temperature, the solvent was removed and resulting residue was partitioned between ethyl acetate (50 mL) and saturated NaHCO₃ (25 mL). The organic layer was separated and the aqueous layer was extracted with ethyl acetate (3×50 mL). The combined organic phases were washed with water, brine, dried over MgSO₄, filtered and concentrated to give a residue, which was purified by MPLC (0 to 60% ethyl acetate in hexanes) to afford product 56-9 (m/z (ES) (M+H)⁺=433).

Step I: 4-{5-chloro-4-methyl-2-[1-methyl-1-(1-methyl-1H-1,2,4-triazol-5-yl)ethyl]phenyl}-1-{[(1R,2R)-2-(2,4-difluorophenyl)-4-methylenecyclo pentyl]carbonyl}piperidine (56-10)

Compound 56-9 (0.070 g, 0.16 mmol) was charged in a 20 mL vial along with methanol (1.5 mL) and HCl (concentrated, 0.5 mL). The mixture was stirred and heated in an oil bath of 40° C. for 20 minutes, and then concentrated by rotary evaporation to afford the amine HCl salt. The HCl salt was charged in a 20 mL round bottomed flask along with acid P-5 (0.042 g, 0.17 mmol), methylene chloride (2 mL) and Hunig's base (0.062 g, 0.49 mmol). The mixture was stirred until the solid dissolved. Then HATU (0.074 g, 0.019 mmol) and HOAT (0.024 g, 0.17 mmol) were added and the resulting reaction mixture was stirred at room temperature for 2 hours. Then the reaction mixture was concentrated to give a residue. The residue was dissolved in methanol (1 mL), filtered through a syringe filtered and washed with methanol (2 mL). The filtrate was concentrated and the resulting residue was purified by RP HPLC(YMC column, 20% to 80% acetonitrile in water) to afford product 56-10 as the TFA salt (m/z (ES) (M+H)⁺=553).

Step J: (3R,4R)-3-[(4-{5-chloro-4-methyl-2-[1-methyl-1-(1-methyl-1H-1,2,4-triazol-5-yl)ethyl]phenyl}piperidin-1-yl)carbonyl]-4-(2,4-difluorophenyl)cyclopentanone (56-11) To a 25 mL one necked round bottomed flask was charged compound 56-10 (0.078 g, 0.141 mmol) and THF/H₂O (1.5/1.5 mL), followed by osmium tetroxide (0.15 mL). The mixture was stirred at room temperature for 30 minutes, then sodium periodate (0.150 g, 0.7 mmol) solution was added dropwise. The resulting mixture was stirred at room temperature for 2 hours, then quenched with 2 mL of saturated Na₂S₂O₃ solution and stirred for 20 minutes. Then the reaction mixture was extracted with ethyl acetate (3×50 mL). The combined organic phases were washed with water, brine, dried over MgSO₄, filtered and concentrated to afford product 56-11, which was used in next step without further purification. (m/z (ES) (M+H)⁺=555).

Step K: N-[(3R,4R)-3-[(4-{5-chloro-4-methyl-2-[1-methyl-1-(1-methyl-1H-1,2,4-triazol-5-yl)ethyl]phenyl}piperidin-1-yl)carbonyl]-4-(2,4-difluorophenyl)-cyclopentyl]-N-methyltetrahydro-2H-pyran-4-amine (56-12) To a 25 mL one necked round bottom flask were charged with compound 56-11 (0.035 g, 0.063 mmol), methylene chloride (2 mL), N-methyl-N-tetrahydro-2H-pyran-4-ylamine (0.095 g, 0.50 mmol), sodium triacetoxyborohydride (0.067 g, 0.32 mmol), molecular sieves (0.10 mg) and triethyl amine (0.064 g, 0.63 mmol). The mixture was stirred at room temperature for 12 hours. The reaction mixture was quenched with MeOH and stirred for 20 minutes. Then the reaction mixture was filtered and concentrated to give a residue, which was purified by RP HPLC(YMC column, 20% to 80% acetonitrile in water) to afford product 56-12 as the TFA salt (m/z (ES) (M+H)⁺=654).

The compounds in Table 7 were prepared using the appropriate reagents following procedures similar to that described above for Example 56 and using the appropriate starting materials:

TABLE 7

| | | Parent Ion |
|---|---|---|
| Example | R¹ | m/z (M + H) |
| 57 | 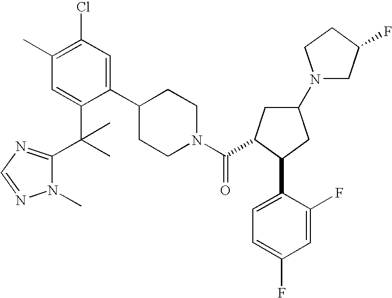 | 628 |
| 58 | 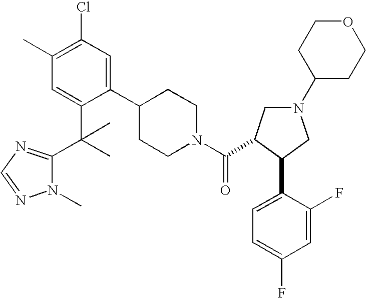 | 626 |

The compounds in Table 8 were prepared using the appropriate reagents following procedures similar to that described above for Example 1 and using the appropriate starting materials:

TABLE 8

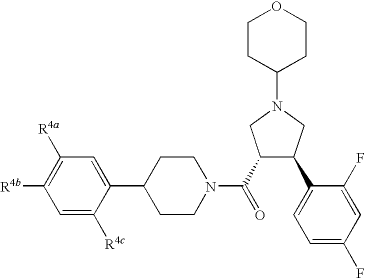

| | | | | Parent Ion m/z |
|---|---|---|---|---|
| Example | R^{4a} | R^{4b} | R^{4c} | (M + H) |
| 59 | Cl | CH₃ | 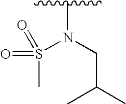 | 652 |
| 60 | Cl | CH₃ | 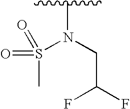 | 660 |

### Example 61

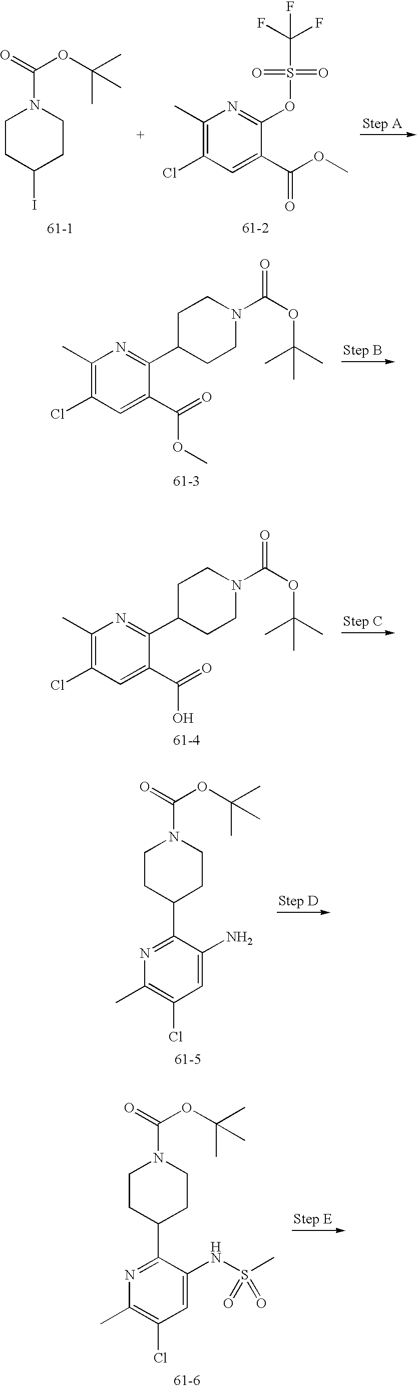

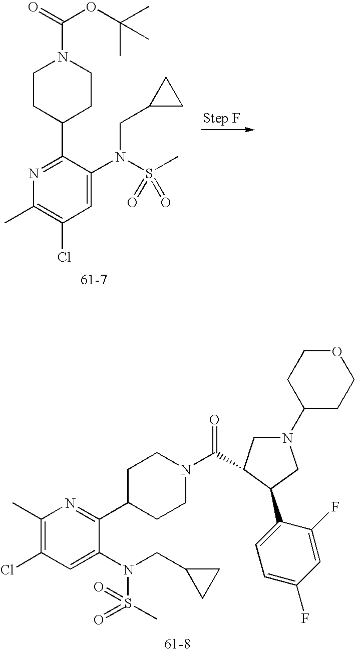

Step A: Methyl 2-[1-(tert-butoxycarbonyl)piperidine-4-yl]-5-chloro-6-methylnicotinate (61-3)

Zinc dust (1.66 g, 25.4 mmol) was suspended in dimethylacetamide (DMA, 4.3 ml) and a solution of trimethylsilyl chloride/1,2-dibromoethane (7:5 w/w, 0.45 ml) was added via syringe over several minutes. The temperature rose to ˜60° C. and stirring was continued for 15 minutes while the reaction mixture cooled back to rt. A solution of the commercially available iodide 61-1 (6.5 g, 20.9 mmol) in DMA (10 ml) was then added from a syringe over 5 minutes. The temperature rose again to ˜66° C. Stirring was continued for 35 min. while the mixture cooled back to rt again, and then filtered through Celite® under nitrogen rinsing the flask and filter with DMA (2.0 ml). This gave a 0.95 M solution in DMA of the zinc iodide insertion product. The triflate 61-2 (2.16 g, 6.46 mmol) (preparation of this material is described in the literature and was obtained from WuXi Pharma Tech) was dissolved in DMA (5.5 ml) and PdCl₂(dppf) catalyst (159 mg, 0.19 mmol) and CuI (74 mg, 0.39 mmol) was added. The mixture was degassed with alternate N₂/high vacuum purges (3×) and a 0.95M solution of the zinc iodide intermediate from above (13.6 ml, 12.9 mmol) was added, then the mixture was heated to 80° C. for 3.5 hr, then then cooled in an ice bath. NH₄C₁—H₂O and ether were added with vigorous stirring. The mixture was filtered through Celite® and washed with water, EtOAc and ether. The layers were separated and extracted the aqueous layer with ether (2×). The combined extracts were washed with brine (1×), dried over MgSO₄, filtered and evaporated. The resulting oil was purified by flash chromatography on silica gel (hexane-ethyl-acetate, 9:1) to give 61-3 as a viscous yellow oil.

Step B: 2-[1-(tert-butoxycarbonyl)piperidine-4-yl]-5-chloro-6-methylnicotinic acid (61-4) The ester 61-3 (356 mg, 1 mmol) was dissolved in methanol (5.0 ml) and 1N NaOH (2.0 ml) was added. The mixture was stirred at rt for 4 hr, then methanol was evaporated and the aqueous residue was neutralized with 1N HCl (2.0 ml), and extracted with EtOAc (3×). The combined extracts washed with brine (1×), dried over MgSO₄, filtered, concentrated and dried under vacuum leaving 61-4 as a foam. ESI-MS Calculated for C₁₇H₂₃ClN₂O₄: 354; Found: 377 [M+Na]⁺.

Step C: tert-butyl 4-(3-amino-5-chloro-6-methylpyridin-2-yl)piperidine-1-carboxylate 61-5 The acid 61-4 (135 mg, 0.381 mmol) and triethyl amine (0.075 ml, 0.54 mmol) were dissolved in acetone (2.0 ml) and cooled in an ice bath. Ethyl chloroformate (0.057 ml, 0.594 mmol) was added and the mixture was stirred for 15 minutes, and then warmed to rt for an additional 50 minutes. A solution of NaN₃ (50 mg, 0.76 mmol) in water (0.100 ml) was added and the mixture was stirred at rt for 45 minutes, then diluted with water and extracted with toluene (3×). The combined organic extracts were washed with brine (1×), dried over MgSO₄ and filtered. Then 2-trimethylsilyethanol (0.109 ml, 0.76 mmol) was added and the mixture was slowly heated to 110° C. for 2 hr, then cooled to rt and stirred overnight. The toluene was evaporated and replaced with acetonitrile (5.0 ml). Then 1M tetrabutyl ammonium fluorinde in THF (0.540 ml, 0.54 mmol) was added and the mixture was heated to 50° C. for 3 hr, followed by cooling and evaporation of the solvent and re dissolving in EtOAc. The EtOAc layers was washed with water-brine (1×) and brine (1×), then dried over MgSO₄, filtered and evaporated. The resulting gum was purified by preparative TLC (silica gel, 20×20 cm plate, 1000μ thickness, hexane-ethyl acetate, 3:1) to give 61-5. ESI-MS Calculated for C₁₆H₂₄ClN₃O₂: 325; Found: 326 [M+H]⁺.

Step D: tert-butyl 4-{5-chloro-6-methyl-3-[(methylsulfonyl)amino]pyridin-2-yl}piperidine-1-carboxlyate (61-6) Amine 61-5 (0.90 g, 0.28 mmol) and methanesulfonyl chloride (0.107 mL, 1.38 mmol) were dissolved in pyridine (2 mL), and the solution heated at 60° C. for 2 h, followed by cooling to rt. The pyridine was removed in vacuo, and replaced with ethyl acetate. The mixture was washed one time each with: water, saturated aqueous NaHCO₃ and brine, and then dried over MgSO₄, and filtered. The resulting filtrate was evaporated in vacuo and the resulting residue was purified by preparative TLC (silica gel, 20×20 cm plate, 1000μ thickness, hexane-ethyl acetate, 3:1) to give 61-6 as a yellow gum. ESI-MS Calculated for C₁₇H₂₆ClN₃O₄S: 403; Found: 404[M+H]⁺.

Step E: tert-butyl 4-{5-chloro-3-[(cyclopropylmethyl)(methylsulfonyl)amino]-6-methylpyridin-2-yl}piperidine-1-carboxylate (61-7) Sulfonamide 61-6 (0.092 g, 0.23 mmol) was dissolved in DMF (1 mL) and a 60% oil dispersion of NaH (0.010 g, 0.25 mmol) was added. The suspension was heated to 60° C. and stirred at this temperature until all solids reacted (about 5 minutes). The mixture was then cooled to rt and bromomethylcyclopropane (0.024 mL, 0.25 mmol) was added. The reaction mixture was stirred at rt for 4.5 hr, then a second portion of NaH (0.10 g, 0.25 mmol) was added and the reaction was heated to 60° C. When the solids had reacted, a second portion of bromomethylcyclopropane (0.050 ml, 0.52 mmol) was added. The mixture was heated for 2.5 hr, then cooled to rt and stirred for 72 hr. The solution was diluted with saturated aqueous NH₄Cl—H₂O, and extracted three times with ethyl acetate. The combined organic extracts were washed with brine, dried over MgSO₄, filtered and the filtrate evaporated to dryness. The resulting residue was purified by preparative TLC (silica gel, 20×20 cm plate, 1000μ thickness, hexane-ethyl acetate, 3:1) to give 61-7.

Step F: N-[5-chloro-2-(1-{[(3S,44R)-4-(2,4-difluorophenyl)-1-(tetrahydro-2H-pyran-4-yl)pyrrolidine-3-yl]carbonyl}piperidine-4-yl)-6-methylpyridin-3-yl]-N-(cyclopropyl-methyl)methanesulfonamide (61-8) 4M HCl in dioxane (3 mL) was added to a solution of BOC protected sulfonamide 61-7 (0.041 g, 0.090 mmol) in dichloromethane (2 mL) and stirred at rt for 1.25 hr. The solvents were evaporated and the residue was briefly dried under vacuum, and then dissolved in dichloromethane (3 mL) with N,N-diisopropylethylamine (0.055 mL, 0.31 mmol). The resulting solution was added to a stirring solution of (3S,4R)-4-(2,4-difluorophenyl)-1-(tetrahydro-2H-pyran-4-yl)pyrrolidine-3-carboxylic acid (0.033 g, 0.108 mmol), 1-hydroxybenzotriazole hydrate (0.017 g, 0.108 mmol) and EDC (0.026 g, 0.134 mmol) in dichloromethane (3 mL). The reaction mixture was stirred overnight at rt, then diluted with dichloromethane and washed one time each with: water, saturated aqueous NaHCO₃ and brine. The organic layer was dried over MgSO₄, filtered and evaporated to give a crude product, which was purified by preparative TLC (silica gel, 20×20 cm plate, 1000μ thickness, hexane-ethyl acetate-MeOH, 12:8:2) giving 61-8. ESI-MS Calculated for C₃₂H₄₁ClF₂N₄O₄S: 650; Found: 651 [M+H]⁺.

The compound in Table 9 was prepared using the appropriate reagents following procedures similar to that described above for Example 61 and using the appropriate starting materials:

TABLE 9

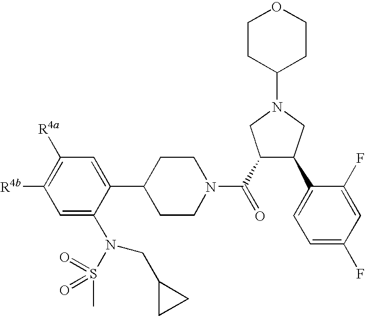

| | | | Parent Ion m/z |
|---|---|---|---|
| Example | R^{4a} | R^{4b} | (M + H) |
| 62 | Cl | CH₃ | 650 |

### Example 63

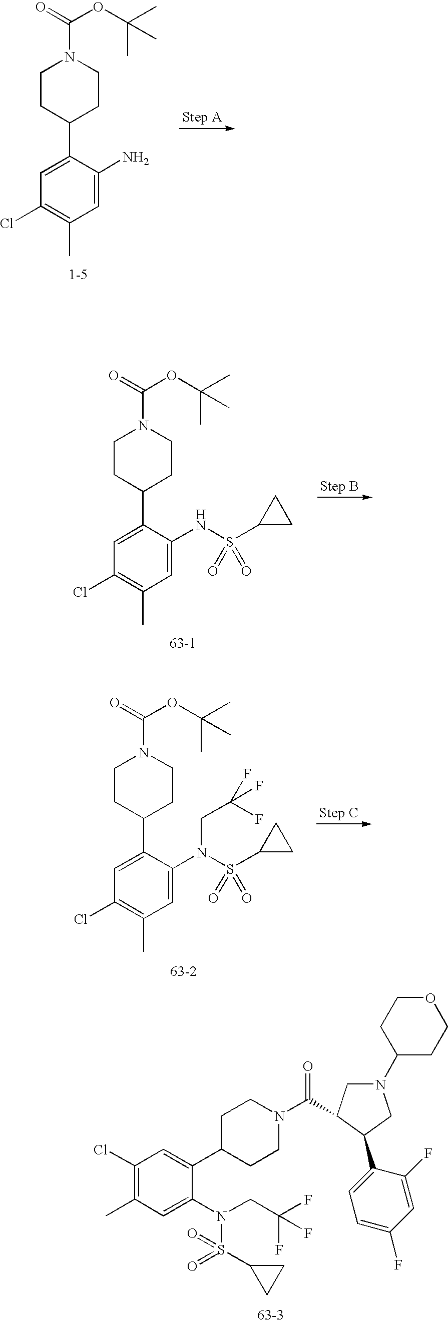

Step A: tert-butyl 4-{5-chloro-2-[(cyclopropylsulfanyl)amino]-4-methylphenyl}piperidine-1-carboxylate (63-1) Aniline 1-5 (0.219 g, 0.675 mmol) and cyclopropanesulfonyl chloride (0.190 mg, 1.35 mmol) were dissolved in pyridine (2 mL), the solution heated at 60° C. for 4 h and then cooled to rt and stirred overnight. The pyridine was removed in vacuo, and replaced with ethyl acetate. The solution was washed one time each with: water, 2N HCl, saturated aqueous NaHCO₃ and brine, then dried over MgSO₄, and filtered. The resulting filtrate was evaporated in vacuo to give 63-1 as an amorphous foam, which was used without further purification. ESI-MS Calculated for C₂₀H₂₉ClN₂O₄S: 428; Found: 429 [M+H]⁺.

Step B: tert-butyl 4-{5-chloro-2-[(cyclopropylsulfanyl)(2,2,2-trifluoroethyl)amino]-4-methylphenyl}piperidine-1-carboxylate (63-2) Sulfonamide 63-1 (0.272 g, 0.635 mmol) was dissolved in DMF (1 mL) and a 60% oil dispersion of NaH (0.051 g, 1.27 mmol) was added. The suspension was heated to 60° C. and stirred at this temperature until all solids reacted (˜5 min). 2,2,2-trifluoroethyl methanesulfonate (0.600 mL, 5.08 mmol) was added, the temperature was raised to 130° C. and the reaction mixture stirred for 20 hr. The solution was cooled to rt, stirred for 24 hr. then diluted with saturated aqueous NH₄Cl—H₂O, and extracted three times with ethyl acetate. The combined organic extracts were washed with brine, dried over MgSO₄, filtered and the filtrate evaporated to dryness. The resulting amber oil was purified by preparative TLC (silica gel, 20×20 cm plate, 1000μ thickness, hexane-ethyl acetate, 3:1) to give 63-2 as an oil. ESI-MS Calculated for C₂₂H₃₀ClF₃N₂O₄S: 510; Found: 511 [M+H]⁺.

Step C: N-[4-chloro-2-(1-{[(3S,4R)-4-(2,4-difluorophenyl)-1-(tetrahydro-2H-1)ran-4-yl)pyrrolidine-3-yl]carbonyl}piperidine-4-yl)-5-methylphenyl]-N-(2,2,2-trifluoroethyl)cyclopropanesulfonamide (63-3)) 4M HCl in dioxane (3 mL) was added to a solution of BOC protected sulfonamide 63-2 (53% pure) (0.607 g, 0.635 mmol max.) in dichloromethane (2 mL) and stirred at rt for 1 hour. The solvents were evaporated and the solid residue was stirred with ether (5 mL) and the ether removed with a pipette. The process was repeated and the residue dried briefly under vacuum giving the BOC deprotected amine hydrochloride salt (145 mg). A portion of this salt (68 mg, 0.152 mmol) was dissolved in dichloromethane (3 mL) with N,N-diisopropylethylamine (0.066 mL, 0.38 mmol). The resulting solution was added to a stirring solution of (3S,4R)-4-(2,4-difluorophenyl)-1-(tetrahydro-2H-pyran-4-yl)pyrrolidine-3-carboxylic acid (0.057 g, 0.183 mmol), 1-hydroxybenzotriazole hydrate (0.028 g, 0.183 mmol) and EDC (0.044 g, 0.223 mmol) in dichloromethane (3 mL). The reaction mixture was stirred overnight at rt, diluted with dichloromethane and washed one time each with water, saturated aqueous NaHCO₃ and brine, dried over MgSO₄, filtered and evaporated. The crude product was purified by preparative TLC (silica gel, 20×20 cm plate, 1000μ, thickness, hexane-ethyl acetate-MeOH, 12:8:2) giving 63-3. ESI-MS Calculated for C₃₃H₃₉ClF₅N₃O₄S: 703; Found: 704 [M+H]⁺.

### Biological Assays

### A. Binding Assay

The membrane binding assay was used to identify competitive inhibitors of ¹²⁵I-NDP-alpha-MSH binding to cloned human MCRs expressed in mouse L- or Chinese hamster ovary (CHO)-cells.

Cell lines expressing melanocortin receptors were grown in T-180 flasks containing selective medium of the composition: 1 L Dulbecco's modified Eagles Medium (DMEM) with 4.5 g L-glucose, 25 mM Hepes, without sodium pyruvate, (Gibco/BR1); 100 mL 10% heat-inactivated fetal bovine serum (Sigma); 10 mL 10,000 unit/mL penicillin & 10,000 μg/mL streptomycin (Gibco/BR1); 10 mL 200 mM L-glutamine (Gibco/BR1); 1 mg/mL geneticin (G418) (Gibco/BR1). The cells were grown at 37° C. with CO₂ and humidity control until the desired cell density and cell number was obtained.

The medium was poured off and 10 mL/monolayer of enzyme-free dissociation media (Specialty Media Inc.) was added. The cells were incubated at 37° C. for 10 min or until cells sloughed off when flask was banged against hand.

The cells were harvested into 200 mL centrifuge tubes and spun at 1000 rpm, 4° C., for 10 min. The supernatant was discarded and the cells were resuspended in 5 mL/monolayer membrane preparation buffer having the composition: 10 mM Tris pH 7.2-7.4; 4 μg/mL Leupeptin (Sigma); 10 μM Phosphoramidon (Boehringer Mannheim); 40 μg/mL Bacitracin (Sigma); 5 μg/mL Aprotinin (Sigma); 10 mM Pefabloc (Boehringer Mannheim). The cells were homogenized with motor-driven dounce (Talboy setting 40), using 10 strokes and the homogenate centrifuged at 6,000 rpm, 4° C., for 15 min.

The pellets were resuspended in 0.2 mL/monolayer membrane prep buffer and aliquots were placed in tubes (500-1000 μL/tube) and quick frozen in liquid nitrogen and then stored at −80° C.

Test compounds or unlabelled NDP-α-MSH was added to 100 μL of membrane binding buffer to a final concentration of 1 μM. The membrane binding buffer had the composition: 50 mM Tris pH 7.2; 2 mM CaCl₂; 1 mM MgCl₂; 5 mM KCl; 0.2% BSA; 4 μg/mL Leupeptin (SIGMA); 10 μM Phosphoramidon (Boehringer Mannheim); 40 μg/mL Bacitracin (SIGMA); 5 μg/mL Aprotinin (SIGMA); and 10 mM Pefabloc (Boehringer Mannheim). One hundred μL of membrane binding buffer containing 10-40 μg membrane protein was added, followed by 100 μM 1251-NDP-α-MSH to final concentration of 100 pM. The resulting mixture was vortexed briefly and incubated for 90-120 min at room temp while shaking.

The mixture was filtered with Packard Microplate 196 filter apparatus using Packard Unifilter 96-well GF/C filter with 0.1% polyethyleneimine (Sigma). The filter was washed (5 times with a total of 10 mL per well) with room temperature of filter wash having the composition: 50 mM Tris-HCl pH 7.2 and 20 mM NaCl. The filter was dried, and the bottom sealed and 50 μL of Packard Microscint-20 was added to each well. The top was sealed and the radioactivity quantitated in a Packard Topcount Microplate Scintillation counter.

### B. Functional Assay

Functional cell based assays were developed to determine the efficacy of agonists and to discriminate melanocortin receptor agonists from antagonists.

Cells (for example, CHO- or L-cells or other eukaryotic cells) expressing a human melanocortin receptor (see e.g. Yang-Y K; Ollmann-M M; Wilson-B D; Dickinson-C; Yamada-T; Barsh-G S; Gantz-I; Mol-Endocrinol. 1997 March; 11(3): 274-80) were dissociated from tissue culture flasks by rinsing with Ca and Mg free phosphate buffered saline (14190-136, Life Technologies, Gaithersburg, Md.) and detached following 5 min incubation at 37° C. with enzyme free dissociation buffer (S-014-B, Specialty Media, Lavellette, N.J.). Cells were collected by centrifugation and resuspended in Earle's Balanced Salt Solution (14015-069, Life Technologies, Gaithersburg, Md.) with additions of 10 mM HEPES pH 7.5, 5 mM MgCl₂, 1 mM glutamine and 1 mg/mL bovine serum albumin. Cells were counted and diluted to 1 to 5×10⁶/mL. The phosphodiesterase inhibitor 3-isobutyl-1-methylxanthine was added to cells to 0.6 mM.

1. Agonist Assay Test compounds were diluted in dimethylsulfoxide (DMSO) (10⁻⁵ to 10⁻¹⁰ M) and 0.1 volume of compound solution was added to 0.9 volumes of cell suspension; the final DMSO concentration was 1%. After room temperature incubation for 45 min, cells were lysed by incubation at 100° C. for 5 min to release accumulated cAMP. cAMP was measured in an aliquot of the cell lysate with the Amersham (Arlington Heights, Ill.) cAMP detection assay (RPA556). The amount of cAMP production which resulted from an unknown compound was compared to that amount of cAMP produced in response to alpha-MSH which was defined as a full agonist with an efficacy of 100%. The EC₅₀ is defined as the compound concentration which results in half maximal stimulation, when compared to its own maximal level of stimulation. Compounds that produce near 0% response are expected to be antagonist which will be further confirmed in the antagonist mode of the functional assay.
2. Antagonist Assay: Antagonist activity was defined as the ability of a compound to block cAMP production in response to alpha-MSH or any agonist. A solution of the test compound and suspension of receptor containing cells were prepared and mixed as described above; the mixture was incubated for 15 min, and an EC₅₀ dose of alpha-MSH (approximately 10 nM alpha-MSH) was added to the cells. The assay was terminated at 45 minutes and cAMP quantitated as above. Percent inhibition was determined by comparing the amount of cAMP produced in the presence to that produced in the absence of test compound. Antagonist is defined as a compound that by itself does not produce agonist-like response, and in combination with an agonist, the compound should inhibit the agonist-induced response.

### C. In Vivo Food Intake and Body Weight Models.

1) Food intake and body weight in rats. Sprague Dawley rats are administered test compound one hour prior to onset of dark cycle (12 hours). Food intake is determined either by measurement of the remaining amount of preweighed food the morning following the dosing or by using a computerized system in which each rat's food is placed on a computer monitored balance. Cumulative food intake for 16 h post compound administration is measured. In some cases, food intake measurements are followed as long as 2 weeks. Body weight is measured daily; in some cases, adiposity is measured by DEXAscan analysis, tissue weights and plasma drug levels are measured. Animals can be dosed by a number of routes of administration. The routes of administration include intravenous, intraperitoneal, subcutaneous and intracerebral ventricular.

Compounds useful in the present invention decrease food intake acutely by at least 20% and/or decrease body weight in a 2 week period by at least 4% relative to placebo.

2) Food intake in diet induced obese mice. Male C57/B16J mice maintained on a high fat diet (30-60% fat calories) are dosed with test compound for 1 to 30 days. Food intake and body weight are measured overnight and sometimes daily as long as 30 days. Biochemical parameters relating to obesity, including leptin, insulin, triglyceride, free fatty acid, cholesterol and serum glucose levels and pharmacokinetic parameters may be determined. Animals can be dosed by a number of routes of administration. The routes of administration include intravenous, intraperitoneal, subcutaneous and intracerebral ventricular. Biochemical parameters relating to obesity, including leptin, insulin, triglyceride, free fatty acid, cholesterol and serum glucose levels are determined.

Compounds useful in the present invention decrease body weight by at least 4% relative to placebo.

### D. Male Sexual Dysfunction: Mouse Electrically Stimulated Cavernosal Nerve (ESCN) Assay

Male C57BL6 mice are anesthetized, the carotid artery is exposed and cannulated for measurement of arterial pressure (MAP). A 30G needle attached to PE10 tubing, filled with heparinized saline, was inserted into the artery and glued in place. This tubing was connected to a pressure transducer and amplifier to measure direct MAP on a Gould 8 channel oscilloscope connected to a computer using the Po-ne-mah software to collect the data at one minute intervals. Another PE10 line attached to a 30 G needle was inserted into the jugular vein for compound or vehicle administration. The cavernous nerve and penile body were exposed through a midline incision. Surrounding muscles were cauterized and removed for visualization of the cavernous nerve, which arises from the ipsilateral pelvic ganglion and is situated dorsal to the prostate. Another 30G needle attached to PE10 tubing, filled with heparinized saline, was inserted into the base of the corpus cavernosum near the crura and connected to the Gould system. A slight increase in intercavernous pressure (ICP) of approximately 5 to 10 mmHg is observed once this cannula is inserted into the corpus cavernosum. Heparinized saline (200 units/mL) was flushed through the cannula to assure proper placement of the cannula, inducing tumescence. The cavernous nerve was then isolated using curved #5 Dumont forceps and placed on a modified fixed position bipolar silver electrode (Harvard Apparatus). The electrodes are encased in plastic to allow stimulation of the nerve without additional stimulation of surrounding tissues. The electrode was advanced and held by a micromanipulator and was attached to a square wave stimulator to deliver electrical impulses at stimulation parameters ranging between 0.5 to 6.0v, 2 to 16 Hz, 1 ms, for 30 seconds. Electrical stimulations were administered to individual animals with 5 minute intervals between stimulations. Responses reported at each time point represent the mean of the two stimulations. ICP, MAP and ICP/MAP responses were continuously recorded at one second intervals for the duration of the experiment.

Measurements of ICP, MAP and ICP/MAP ratio are analyzed and responses compared to nerve stimulation in the presence and absence of compound or vehicle. For each parameter monitored, responses evoked by duplicate electrical stimulations were averaged, and the mean values were used for comparison. Response segments of 10 s of baseline+30 s stimulation+150 s post-stimulation were used to evaluate changes in ICP in response to electrical stimulation of the cavernous nerve. To assess direct effects of compound administration on ICP, a 300 s pre-compound response segment was compared to a comparable segment immediately after compound administration.

Compounds useful in the present invention increase intracavernous pressure by at least 25% for a time period of at least 15 minutes relative to placebo.

### E. Models of Female Sexual Dysfunction

Rodent assays relevant to female sexual receptivity include the behavioral model of lordosis and direct observations of copulatory activity. There is also an urethrogenital reflex model in anesthetized spinally transected rats for measuring orgasm in both male and female rats. These and other established animal models of female sexual dysfunction are described in McKenna K E et al, A Model For The Study of Sexual Function In Anesthetized Male And Female Rats, Am. J. Physiol. (Regulatory Integrative Comp. Physiol 30): R1276-R1285, 1991; McKenna K E et al, Modulation By Peripheral Serotonin of The Threshold For Sexual Reflexes In Female Rats, Pharm. Bioch. Behav., 40:151-156, 1991; and Takahashi L K et al, Dual Estradiol Action In The Diencephalon And The Regulation Of Sociosexual Behavior In Female Golden Hamsters, Brain Res., 359:194-207, 1985.

### F. Model of Cachexia

Rodent assays relevant to cachexia include the tumor cachexia model, in which cells derived from a tumor were injected into mice. Over a period of 1-3 weeks, a tumor will form and grow in the implanted mice. Tumor-bearing mice will exhibit reduced food intake and reduced body weight. By treating the tumor-bearing mice with an effective MC4R antagonist, food intake will be increased and body weight will be increased. This animal model of cachexia is described in Cone, R. D. et al, Role of the Central Melanocortin System in Cachexia, Cancer Research 61, 1432-38, Feb. 15, 2001.

The compounds of the present invention, including Examples 1-63, were tested and found to bind to the melanocortin-4 receptor with IC₅₀ values less than 10 μM. The agonist compounds of the present invention, including Examples 1-63, were also tested in the functional assay and found to activate the melanocortin-4 receptor with EC₅₀ values less than 5 μM. The antagonist compounds of the present invention were tested in the functional assay and found not to activate the melanocortin-4 receptor with an efficacy <5%, and have an IC₅₀ from the antagonist assay of less than 10 uM.

### Examples of Pharmaceutical Compositions

As a specific embodiment of an oral composition of a composition of the present invention, 5 mg of Example 1 is formulated with sufficient finely divided lactose to provide a total amount of 580 to 590 mg to fill a size O hard gelatin capsule.

As another specific embodiment of an oral composition of a compound of the present invention, 2.5 mg of Example 1 is formulated with sufficient finely divided lactose to provide a total amount of 580 to 590 mg to fill a size O hard gelatin capsule.

While the invention has been described and illustrated in reference to certain preferred embodiments thereof, those skilled in the art will appreciate that various changes, modifications and substitutions can be made therein without departing from the spirit and scope of the invention. For example, effective dosages other than the preferred doses as set forth hereinabove may be applicable as a consequence of variations in the responsiveness of the subject or mammal being treated for obesity, diabetes, obesity related disorders, or for other indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and embodiments of the present invention. It is intended, therefore, that the invention be limited only by the scope of the claims which follow and that such claims be interpreted as broadly as is reasonable.

1. A compound of structural formula I: 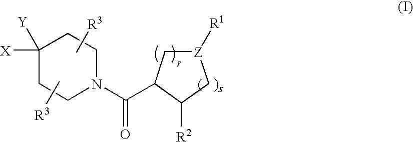
or a pharmaceutically acceptable salt thereof; wherein
X is selected from the group consisting of:
(1) —C₁₋₈ alkyl,
(2) —(CH₂)ₙC₃₋₈ cycloalkyl,
(3) —(CH₂)ₙ-phenyl,
(4) —(CH₂)ₙ-naphthyl,
(5) —(CH₂)ₙ-heteroaryl,
(6) —(CH₂)ₙheterocycloalkyl,
(7) —(CH₂)ₙC≡N,
(8) —(CH₂)ₙCON(R⁵)₂,
(9) —(CH₂)ₙCO₂R⁵,
(10) —(CH₂)ₙCOR⁵,
(11) —(CH₂)ₙNR⁵C(O)R⁵,
(12) —(CH₂)ₙNR⁵CO₂R⁵,
(13) —(CH₂)ₙNR⁵C(O)N(R⁵)₂,
(14) —(CH₂)ₙNR⁵SO₂R⁵,
(15) —(CH₂)ₙS(O)ₚR⁵,
(16) —(CH₂)ₙSO₂N(R⁵)₂,
(17) —(CH₂)ₙOR⁵,
(18) —(CH₂)ₙOC(O)R⁵,
(19) —(CH₂)ₙOC(O)OR⁵,
(20) —(CH₂)ₙOC(O)N(R⁵)₂,
(21) —(CH₂)ₙN(R⁵)₂, and
(22) —(CH₂)ₙNR⁵SO₂N(R⁵)₂,
wherein phenyl, naphthyl, and heteroaryl are unsubstituted or substituted with one to three groups independently selected from R⁴, and alkyl, cycloalkyl, and heterocycloalkyl are unsubstituted or substituted with one to three groups independently selected from R⁴ and oxo, and wherein any (CH₂) in X is unsubstituted or substituted with one to two groups independently selected from halogen, hydroxy, and —C₁₋₆alkyl;
Y is selected from the group consisting of:
(1) hydrogen,
(2)-C₁₋₈ alkyl,
(3) —C₂₋₆ alkenyl,
(4) —(CH₂)ₙC₃₋₈ cycloalkyl,
(5) —(CH₂)ₙ-phenyl,
(6) —(CH₂)ₙ-naphthyl,
(7) —(CH₂)ₙ-heteroaryl, and
(8) —(CH₂)ₙ-heterocycloalkyl,
wherein alkenyl, phenyl, naphthyl, and heteroaryl are unsubstituted or substituted with one to three groups independently selected from R⁴, and alkyl, cycloalkyl, and heterocycloalkyl are optionally substituted with one to three groups independently selected from R⁴ and oxo, and wherein any (CH₂) in Y is unsubstituted or substituted with one to two groups independently selected from halogen, hydroxy, and —C₁₋₆alkyl;
Z is selected from the group consisting of:
(1) —CH—, and
(2) —N—;
R¹ is selected from the group consisting of:
(1) —(CH₂)ₙC₂₋₇heterocycloalkyl,
(2) —(CH₂)ₙbridgedC₂₋₇heterocycloalkyl, and
(3) —N(R⁷)C₂₋₇heterocycloalkyl,
wherein heterocycloalkyl and (CH₂)ₙ are unsubstituted or substituted with one to three groups independently selected from R⁹ and oxo, provided that Z and R¹ are not attached via a N—N bond;
R² is selected from the group consisting of:
(1) phenyl,
(2) naphthyl, and
(3) heteroaryl,
wherein phenyl, naphthyl, and heteroaryl are unsubstituted or substituted with one to four groups independently selected from R⁸;
each R³ is independently selected from the group consisting of:
(1) hydrogen,
(2) —OH,
(3) —C₁₋₈alkyl,
(4) —OC₁₋₈alkyl,
(5) halogen,
(6) —N(R⁵)₂,
(7) —SR⁵, and
(8) —CF₃,
wherein two C₁₋₈alkyl substituents along with the atoms to which they are attached can form a 4- to 8-membered cycloalkyl or heterocycloalkyl ring, and provided that when Z is —N—, Y is H or —OH, X is phenyl substituted with one to three R⁴ substituents and at least one R⁴ is —C₁₋₄alkyl, —(CH₂)₀₋₂C₃₋₅ cycloalkyl, halogen, —(CH₂)₀₋₃OR^{a}, CN, CO₂R^{b}, —(CH₂)₀₋₂NR^{b}SO₂R^{c}C, CF₃, CH₂CF₃, OCF₃, or OCH₂CF₃, wherein R^{a}, R^{b} and R^{c}C are —H, —CH₃, or —CH₂CH₃, then both R³ substituents are not methyl;
each R⁴ is independently selected from the group consisting of:
(1) —C₁₋₈alkyl,
(2) —C₂₋₈ alkenyl,
(3) —(CH₂)ₙ-phenyl,
(4) —(CH₂)ₙ-naphthyl,
(5) —(CH₂)ₙ-heteroaryl,
(6) —(CH₂)ₙC₂₋₇ heterocycloalkyl,
(7) —(CH₂)ₙC₃₋₇ cycloalkyl,
(8) —(CH₂)ₙ-halogen,
(9) —(CH₂)ₙ—OR⁶,
(10) —(CH₂)ₙ—OSi(C₁₋₆alkyl)₃,
(11) —(CH₂)ₙC(O)R⁶,
(12) —(CH₂)ₙOC(O)R⁶,
(13) —(CH₂)ₙC(O)OR⁶,
(14) —(CH₂)ₙC≡N,
(15) —NO₂,
(16) —(CH₂)ₙN(R⁶)₂,
(17) —(CH₂)ₙC(O)N(R⁶)₂,
(18) —(CH₂)ₙNR⁶C(O)R⁶,
(19) —(CH₂)ₙNR⁶C(O)OR⁶,
(20) —(CH₂)ₙNR⁶C(O)-heteroaryl,
(21) —(CH₂)ₙNR⁶C(O)N(R⁶)₂,
(22) —(CH₂)ₙC(O)NR⁶N(R⁶)₂,
(23) —(CH₂)ₙC(O)NR⁶NR⁶C(O)R⁶,
(24) —(CH₂)ₙNR⁶S(O)ₚR⁶,
(25) —(CH₂)ₙS(O)ₚN(R⁶)₂,
(26) —(CH₂)ₙS(O)ₚR⁶,
(27) —O(CH₂)ₙC(O)N(R⁶)₂,
(28) —CF₃,
(29) —CH₂CF₃,
(30) —OCF₃, and
(31) —OCH₂CF₃,
wherein alkenyl, phenyl, naphthyl, heteroaryl are unsubstituted or substituted with one to three substituents independently selected from halogen, hydroxy, —C₁₋₆alkyl, trifluoromethyl, —C₁₋₆alkoxy, —CO₂C₁₋₆alkyl, and —CO₂H, and wherein any alkyl, cycloalkyl, and heterocycloalkyl in R⁴ is unsubstituted or substituted with one or two groups independently selected from halogen, hydroxy, oxo, —C₁₋₆alkyl, trifluoromethyl, —C₁₋₆alkoxy, —CO₂C₁₋₆alkyl, and —CO₂H, or two R⁴ substituents on the same carbon atom are taken together with the carbon atom to form a cyclopropyl group;
R⁵ is independently selected from the group consisting of
(1) hydrogen,
(2) —C₁₋₈alkyl,
(3) —C₂₋₈alkenyl,
(4) —C₂₋₈alkynyl,
(5) —C₁₋₈alkoxy,
(6) —(CH₂)ₙC₃₋₇cycloalkyl,
(7) —(CH₂)ₙC₂₋₇heterocycloalkyl,
(8) —(CH₂)ₙ-phenyl,
(9) —(CH₂)ₙ-naphthyl,
(10) —(CH₂)ₙ-heteroaryl, and
(11) —(CH₂)ₙC₃₋₇bicycloalkyl,
wherein alkenyl, alkynyl, phenyl, naphthyl, and heteroaryl are unsubstituted or substituted with one to three groups independently selected from R⁴, and alkyl, alkoxy, cycloalkyl, heterocycloalkyl, and bicycloalkyl are unsubstituted or substituted with one to three groups independently selected from R⁴ and oxo, and wherein any (CH₂) in R⁵ is unsubstituted or substituted with one to two groups independently selected from halogen, hydroxy, and —C₁₋₆alkyl, or two R⁵ groups together with the atom to which they are attached form a 5- to 8-membered mono- or bicyclic ring system optionally containing an additional heteroatom selected from O, S, and —NC₁₋₄ alkyl;
each R⁶ is independently selected from the group consisting of:
(1) hydrogen,
(2) —C₁₋₆ alkyl,
(3) —(CH₂)ₙ-phenyl,
(4) —(CH₂)ₙ-heteroaryl,
(5) —(CH₂)ₙ-naphthyl,
(6) —(CH₂)ₙ-heterocycloalkyl,
(7) —(CH₂)ₙC₃₋₇cycloalkyl,
(8) —(CH₂)ₙC₃₋₇bicycloalkyl,
(9) —(CH₂)ₙCF₃, and
(10) —(CH₂)ₙCHF₂,
wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy, or two R⁶ groups together with the atom to which they are attached form a 4- to 8-membered mono- or bicyclic ring system optionally containing an additional heteroatom selected from O, S, and NC₁₋₄ alkyl;
each R⁷ is independently selected from the group consisting of:
(1) hydrogen, and
(2) —C₁₋₈alkyl,
wherein alkyl is unsubstituted or substituted with one to three groups independently selected from halogen, —C₁₋₆alkyl, hydroxy, and C₁₋₄ alkoxy;
each R⁸ is independently selected from the group consisting of:
(1) —C₁₋₆alkyl,
(2) —(CH₂)ₙphenyl,
(3) —(CH₂)ₙnaphthyl,
(4) —(CH₂)ₙheteroaryl,
(5) —(CH₂)ₙC₂₋₇heterocycloalkyl,
(6) —(CH₂)ₙC₃₋₇cycloalkyl,
(7) halogen,
(8) —OR⁶,
(9) —(CH₂)ₙN(R⁶)₂,
(10) —(CH₂)ₙC≡N,
(11) —(CH₂)ₙCO₂R⁶,
(12) —NO₂,
(13) —(CH₂)ₙNR⁶S(O)ₚR⁶,
(14) —(CH₂)ₙS(O)ₚN(R⁶)₂,
(15) —(CH₂)ₙS(O)ₚR⁶,
(16) —(CH₂)ₙNR⁶C(O)N(R⁶)₂,
(17) —(CH₂)ₙC(O)N(R⁶)₂,
(18) —(CH₂)ₙNR⁶C(O)R⁶,
(19) —(CH₂)ₙNR⁶CO₂R⁶,
(20) —(CH₂)ₙNR⁶C(O)-heteroaryl,
(21) —(CH₂)ₙC(O)NR⁶N(R⁶)₂,
(22) —(CH₂)ₙC(O)NR⁶NR⁶C(O)R⁶,
(23) —O(CH₂)ₙC(O)N(R⁶)₂,
(24) —CF₃,
(25) —CH₂CF₃,
(26) —OCF₃, and
(27) —OCH₂CF₃,
wherein phenyl, naphthyl, and heteroaryl are unsubstituted or substituted with one to three substituents independently selected from halogen, hydroxy, —C₁₋₆alkyl, trifluoromethyl, and C₁₋₄ alkoxy, and wherein alkyl, cycloalkyl, and heterocycloalkyl are unsubstituted or substituted with one to three substituents independently selected from halogen, hydroxy, oxo, —C₁₋₆alkyl, trifluoromethyl, and C₁₋₄ alkoxy;
each R⁹ is independently selected from the group consisting of:
(1) —(CH₂)ₙ-halogen,
(2) —C₁₋₆alkyl,
(3) —(CH₂)ₙ—CO₂R⁶,
(4) —(CH₂)ₙ—OR⁶,
(5) —(CH₂)ₙ-phenyl,
(6) —(CH₂)ₙ-heteroaryl,
(7) —(CH₂)ₙ-naphthyl,
(8) —(CH₂)ₙ-heterocycloalkyl,
(9)-(CH₂)ₙC₃₋₇cycloalkyl,
(10) —(CH₂)ₙC₃₋₇bicycloalkyl,
(11) —(CH₂)ₙCF₃, and
(12) —(CH₂)ₙCHF₂,
wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy, or two R⁶ groups together with the atom to which they are attached form a 4- to 8-membered mono- or bicyclic ring system optionally containing an additional heteroatom selected from O, S, and NC₁₋₄ alkyl;
r is 1 or 2;
s is 0, 1, or 2;
n is 0, 1, 2, 3, or 4; and
p is 0, 1, or 2.

2. The compound of claim 1 wherein each R³ is hydrogen; or a pharmaceutically acceptable salt thereof.

3. The compound of claim 1 wherein R² is phenyl substituted with one to four groups independently selected from R⁸; or a pharmaceutically acceptable salt thereof.

4. The compound of claim 1 wherein X is selected from the group consisting of: -phenyl, -pyridyl and —(CH₂)ₙCON(R⁵)₂, wherein phenyl and pyridyl are unsubstituted or substituted with one to three groups independently selected from R⁴, and any (CH₂) in X is unsubstituted or substituted with one to two groups independently selected from halogen, hydroxy, and —C₁₋₆alkyl; or a pharmaceutically acceptable salt thereof.

5. The compound of claim 1 wherein R⁴ is independently selected from the group consisting of: —C₁₋₈ alkyl, —(CH₂)ₙ-heteroaryl, —(CH₂)ₙ-halogen, —(CH₂)ₙNR⁶C(O)R⁶, and —(CH₂)ₙNR⁶S(O)ₚR⁶, wherein heteroaryl is unsubstituted or substituted with one to three substituents independently selected from halogen, hydroxy, —C₁₋₆alkyl, trifluoromethyl, —C₁₋₆alkoxy, —CO₂C₁₋₆alkyl, and —CO₂H, and wherein any alkyl in R⁴ is unsubstituted or substituted with one to two groups independently selected from halogen, hydroxy, oxo, —C₁₋₆alkyl, trifluoromethyl, —C₁₋₆alkoxy, —CO₂C₁₋₆alkyl; or a pharmaceutically acceptable salt thereof.

6. The compound of claim 1 wherein Y is hydrogen, and X is phenyl or pyridyl, wherein phenyl and pyridyl are substituted with one to three groups independently selected from R⁴; or a pharmaceutically acceptable salt thereof.

7. The compound of claim 1, wherein Y is cyclohexane and X is —C(O)NHC(CH₃)₃; or a pharmaceutically acceptable salt thereof.

8. The compound of claim 1 wherein Z is —CH—; or a pharmaceutically acceptable salt thereof.

9. The compound of claim 1 wherein Z is —N—; or a pharmaceutically acceptable salt thereof.

10. The compound of claim 1 wherein r is 1 and s is 1.

11. The compound of claim 1 of structural formula IIa or IIb of the indicated trans relative stereochemical configuration: 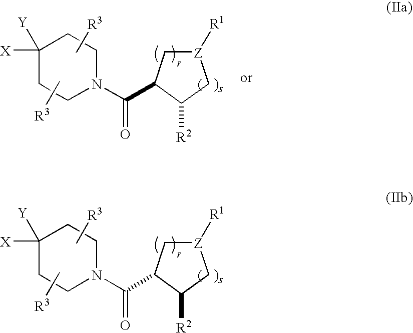
or a pharmaceutically acceptable salt thereof; wherein
X is selected from the group consisting of
(1) —C₁₋₈ alkyl,
(2) —(CH₂)ₙC₃₋₈ cycloalkyl,
(3) —(CH₂)ₙ-phenyl,
(4) —(CH₂)ₙ-naphthyl,
(5) —(CH₂)ₙ-heteroaryl,
(6) —(CH₂)ₙheterocycloalkyl,
(7) —(CH₂)ₙC≡N,
(8) —(CH₂)ₙCON(R⁵)₂,
(9) —(CH₂)ₙCO₂R⁵,
(10) —(CH₂)ₙCOR⁵,
(11) —(CH₂)ₙNR⁵C(O)R⁵,
(12) —(CH₂)ₙNR⁵CO₂R⁵,
(13) —(CH₂)ₙNR⁵C(O)N(R⁵)₂,
(14) —(CH₂)ₙNR⁵SO₂R⁵,
(15) —(CH₂)ₙS(O)ₚR⁵,
(16) —(CH₂)ₙSO₂N(R⁵)₂,
(17) —(CH₂)ₙOR⁵,
(18) —(CH₂)ₙOC(O)R⁵,
(19) —(CH₂)ₙOC(O)OR⁵,
(20) —(CH₂)ₙOC(O)N(R⁵)₂,
(21) —(CH₂)ₙN(R⁵)₂, and
(22) —(CH₂)ₙNR⁵SO₂N(R⁵)₂,
wherein phenyl, naphthyl, and heteroaryl are unsubstituted or substituted with one to three groups independently selected from R⁴, and alkyl, cycloalkyl, and heterocycloalkyl are unsubstituted or substituted with one to three groups independently selected from R⁴ and oxo, and wherein any (CH₂) in X is unsubstituted or substituted with one to two groups independently selected from halogen, hydroxy, and —C₁₋₆alkyl;
Y is selected from the group consisting of:
(1) hydrogen,
(2) —C₁₋₈ alkyl,
(3) —C₂₋₆ alkenyl,
(4) —(CH₂)ₙC₃₋₈ cycloalkyl,
(5) —(CH₂)ₙ-phenyl,
(6) —(CH₂)ₙ-naphthyl,
(7) —(CH₂)ₙ-heteroaryl, and
(8) —(CH₂)ₙ-heterocycloalkyl,
wherein alkenyl, phenyl, naphthyl, and heteroaryl are unsubstituted or substituted with one to three groups independently selected from R⁴, alkyl, cycloalkyl, and heterocycloalkyl are optionally substituted with one to three groups independently selected from R⁴ and oxo, and wherein any (CH₂) in Y is unsubstituted or substituted with one to two groups independently selected from halogen, hydroxy, and —C₁₋₆alkyl;
Z is selected from the group consisting of:
(1) —CH—, and
(2) —N—;
R¹ is selected from the group consisting of:
(1) —(CH₂)ₙC₂₋₇heterocycloalkyl,
(2) —(CH₂)ₙbridgedC₂₋₇heterocycloalkyl, and
(3) —N(R⁷)C₂₋₇heterocycloalkyl,
wherein heterocycloalkyl and (CH₂)ₙ are unsubstituted or substituted with one to three groups independently selected from R⁹ and oxo, provided that Z and R¹ are not attached via a N—N bond;
R² is selected from the group consisting of:
(1) phenyl,
(2) naphthyl, and
(3) heteroaryl,
wherein phenyl, naphthyl, and heteroaryl are unsubstituted or substituted with one to four groups independently selected from R⁸;
each R³ is independently selected from the group consisting of:
(1) hydrogen,
(2) —OH,
(3) —C₁₋₈alkyl,
(4) —OC₁₋₈alkyl,
(5) halogen,
(6) —N(R⁵)₂,
(7) —SR⁵, and
(8) —CF₃,
wherein two C₁₋₈alkyl substituents along with the atoms to which they are attached can form a 4- to 8-membered cycloalkyl or heterocycloalkyl ring, and provided that when Z is —N—, Y is H or —OH, X is phenyl substituted with one to three R⁴ substituents and at least one R⁴ is C₁₋₄alkyl, —(CH₂)₀₋₂C₃₋₅ cycloalkyl, halogen, —(CH₂)₀₋₃OR^{a}, CN, CO₂R^{b}, —(CH₂)₀₋₂NR^{b}SO₂R^{c}, CF₃, CH₂CF₃, OCF₃, or OCH₂CF₃, wherein R^{a}, R^{b} and R^{c} are —H, —CH₃, or —CH₂CH₃, then both R³ substituents are not methyl;
each R⁴ is independently selected from the group consisting of:
(1)-C₁₋₈ alkyl,
(2) —C₂₋₈ alkenyl,
(3) —(CH₂)ₙ-phenyl,
(4) —(CH₂)ₙ-naphthyl,
(5) —(CH₂)ₙ-heteroaryl,
(6) —(CH₂)ₙC₂₋₇ heterocycloalkyl,
(7) —(CH₂)ₙC₃₋₇ cycloalkyl,
(8) —(CH₂)ₙ-halogen,
(9) —(CH₂)ₙ—OR⁶,
(10) —(CH₂)ₙ—OSi(C₁₋₆alkyl)₃,
(11) —(CH₂)ₙC(O)R⁶,
(12) —(CH₂)ₙOC(O)R⁶,
(13) —(CH₂)ₙC(O)OR⁶,
(14) —(CH₂)ₙC═N,
(15) —NO₂,
(16) —(CH₂)ₙN(R⁶)₂,
(17) —(CH₂)ₙC(O)N(R⁶)₂,
(18) —(CH₂)ₙNR⁶C(O)R⁶,
(19) —(CH₂)ₙNR⁶C(O)OR⁶,
(20) —(CH₂)ₙNR⁶C(O)-heteroaryl,
(21) —(CH₂)ₙNR⁶C(O)N(R⁶)₂,
(22) —(CH₂)ₙC(O)NR⁶N(R⁶)₂,
(23) —(CH₂)ₙC(O)NR⁶NR⁶C(O)R⁶,
(24) —(CH₂)ₙNR⁶S(O)ₚR⁶,
(25) —(CH₂)ₙS(O)ₚN(R⁶)₂,
(26) —(CH₂)ₙS(O)ₚR⁶,
(27) —O(CH₂)ₙC(O)N(R⁶)₂,
(28) —CF₃,
(29) —CH₂CF₃,
(30) —OCF₃, and
(31) —OCH₂CF₃,
wherein alkenyl, phenyl, naphthyl, heteroaryl are unsubstituted or substituted with one to three substituents independently selected from halogen, hydroxy, —C₁₋₆alkyl, trifluoromethyl, —C₁₋₆alkoxy, —CO₂C₁₋₆alkyl, and —CO₂H, and wherein any alkyl, cycloalkyl, and heterocycloalkyl in R⁴ is unsubstituted or substituted with one to two groups independently selected from halogen, hydroxy, oxo, —C₁₋₆alkyl, trifluoromethyl, —C₁₋₆alkoxy, —CO₂C₁₋₆alkyl, and —CO₂H, or two R⁴ substituents on the same carbon atom are taken together with the carbon atom to form a cyclopropyl group;
R⁵ is independently selected from the group consisting of:
(1) hydrogen,
(2) —C₁₋₈alkyl,
(3) —C₂₋₈alkenyl,
(4) —C₂₋₈alkynyl,
(5) —C₁₋₈alkoxy,
(6) —(CH₂)ₙC₃₋₇cycloalkyl,
(7) —(CH₂)ₙC₂₋₇heterocycloalkyl,
(8) —(CH₂)ₙ-phenyl,
(9) —(CH₂)ₙ-naphthyl,
(10) —(CH₂)ₙ-heteroaryl, and
(11) —(CH₂)ₙC₃₋₇bicycloalkyl,
wherein alkenyl, alkynyl, phenyl, naphthyl, and heteroaryl are unsubstituted or substituted with one to three groups independently selected from R⁴, and alkyl, alkoxy, cycloalkyl, heterocycloalkyl, and bicycloalkyl are unsubstituted or substituted with one to three groups independently selected from R⁴ and oxo, and wherein any (CH₂) in R⁵ is unsubstituted or substituted with one to two groups independently selected from halogen, hydroxy, and —C₁₋₆alkyl, or two R⁵ groups together with the atom to which they are attached form a 5- to 8-membered mono- or bicyclic ring system optionally containing an additional heteroatom selected from O, S, and —NC₁₋₄ alkyl;
each R⁶ is independently selected from the group consisting of:
(1) hydrogen,
(2) —C₁₋₆ alkyl,
(3) —(CH₂)ₙ-phenyl,
(4) —(CH₂)ₙ-heteroaryl,
(5) —(CH₂)ₙ-naphthyl,
(6) —(CH₂)ₙ-heterocycloalkyl,
(7) —(CH₂)ₙC₃₋₇cycloalkyl,
(8) —(CH₂)ₙC₃₋₇bicycloalkyl,
(9) —(CH₂)ₙCF₃, and
(10) —(CH₂)ₙCHF₂,
wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy, or two R⁶ groups together with the atom to which they are attached form a 4- to 8-membered mono- or bicyclic ring system optionally containing an additional heteroatom selected from O, S, and NC₁₋₄ alkyl;
each R⁷ is independently selected from the group consisting of:
(1) hydrogen, and
(2) —C₁₋₈alkyl,
wherein alkyl is unsubstituted or substituted with one to three groups independently selected from halogen, —C₁₋₆alkyl, hydroxy, and C₁₋₄ alkoxy;
each R⁸ is independently selected from the group consisting of:
(1) —C₁₋₆alkyl,
(2) —(CH₂)ₙphenyl,
(3) —(CH₂)ₙnaphthyl,
(4) —(CH₂)ₙheteroaryl,
(5) —(CH₂)ₙC₂₋₇heterocycloalkyl,
(6) —(CH₂)ₙC₃₋₇cycloalkyl,
(7) halogen,
(8) —OR⁶,
(9) —(CH₂)ₙN(R⁶)₂,
(10) —(CH₂)ₙC═N,
(11) —(CH₂)ₙCO₂R⁶,
(12) —NO₂,
(13) —(CH₂)ₙNR⁶S(O)ₚR⁶,
(14) —(CH₂)ₙS(O)ₚN(R⁶)₂,
(15) —(CH₂)ₙS(O)ₚR⁶,
(16) —(CH₂)ₙNR⁶C(O)N(R⁶)₂,
(17) —(CH₂)ₙC(O)N(R⁶)₂,
(18) —(CH₂)ₙNR^{6c}(O)R⁶,
(19) —(CH₂)ₙNR^{6c}O₂R⁶,
(20) —(CH₂)ₙNR⁶C(O)-heteroaryl,
(21) —(CH₂)ₙC(O)NR⁶N(R⁶)₂,
(22) —(CH₂)ₙC(O)NR⁶NR⁶C(O)R⁶,
(23) —O(CH₂)ₙC(O)N(R⁶)₂,
(24) —CF₃,
(25) —CH₂CF₃,
(26) —OCF₃, and
(27) —OCH₂CF₃,
wherein phenyl, naphthyl, and heteroaryl are unsubstituted or substituted with one to three substituents independently selected from halogen, hydroxy, —C₁₋₆alkyl, trifluoromethyl, and C₁₋₄ alkoxy, and wherein alkyl, cycloalkyl, heterocycloalkyl and (CH₂) are unsubstituted or substituted with one to three substituents independently selected from halogen, hydroxy, oxo, —C₁₋₆alkyl, trifluoromethyl, and C₁₋₄ alkoxy;
each R⁹ is independently selected from the group consisting of:
(1) —(CH₂)ₙ-halogen,
(2) —C₁₋₆alkyl,
(3) —(CH₂)ₙ—CO₂R⁶,
(4) —(CH₂)ₙ—OR⁶,
(5) —(CH₂)ₙ-phenyl,
(6) —(CH₂)ₙ-heteroaryl,
(7) —(CH₂)ₙ-naphthyl,
(8) —(CH₂)ₙ-heterocycloalkyl,
(9) —(CH₂)ₙC₃₋₇cycloalkyl,
(10) —(CH₂)ₙC₃₋₇bicycloalkyl,
(11) —(CH₂)ₙCF₃, and
(12) —(CH₂)ₙCHF₂,
wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy, or two R⁶ groups together with the atom to which they are attached form a 4- to 8-membered mono- or bicyclic ring system optionally containing an additional heteroatom selected from O, S, and NC₁₋₄ alkyl;
r is 1 or 2;
s is 0, 1, or 2;
n is 0, 1, 2, 3, or 4; and
p is 0, 1, or 2.

12. The compound of claim 1 of the following structural formula IIa and IIb with the indicated trans relative stereochemical configuration: 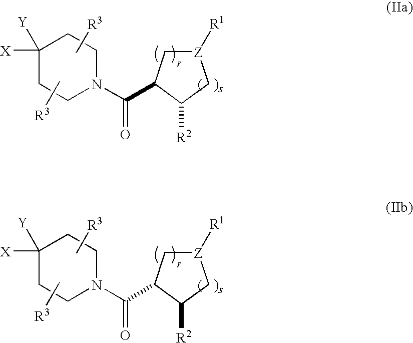
or a pharmaceutically acceptable salt thereof; wherein
X is selected from the group consisting of:
(1) phenyl, and
(2) pyridyl,
wherein phenyl and pyridyl are unsubstituted or substituted with one to three groups independently selected from R⁴;
Y is hydrogen;
Z is selected from the group consisting of:
(1) —CH—, and
(2) —N—;
R¹ is selected from the group consisting of:
(1) tetrahydropyran,
(2) pyrrolidine,
(3) 2-oxa-5-azabicyclo[2.2.1]heptane, and
(4) —N(CH₃)-tetrahydropyran,
wherein R¹ is unsubstituted or substituted with one to three groups independently selected from R⁴ and oxo wherein heterocycloalkyl and (CH₂)ₙ are unsubstituted or substituted with one to three groups independently selected from R⁹ and oxo, provided that Z and R¹ are not attached via a N—N bond.
R² is 2,4-difluorophenyl;
each R³ is hydrogen;
each R⁴ is independently selected from the group consisting of:
(1) —C₁₋₈ alkyl,
(2) —(CH₂)ₙ-heteroaryl,
(3) —(CH₂)ₙ-halogen,
(4) —(CH₂)ₙNR⁶C(O)R⁶, and
(5) —(CH₂)ₙNR⁶S(O)ₚR⁶,
wherein heteroaryl are unsubstituted or substituted with one to three substituents independently selected from halogen, hydroxy, —C₁₋₆alkyl, trifluoromethyl, —C₁₋₆alkoxy, —CO₂C₁₋₆alkyl, and —CO₂H, and wherein any alkyl in R⁴ is unsubstituted or substituted with one to two groups independently selected from halogen, hydroxy, oxo, —C₁₋₆alkyl, trifluoromethyl, —C₁₋₆alkoxy, and —CO₂C₁₋₆alkyl;
R⁵ is independently selected from the group consisting of:
(1) hydrogen,
(2) —C₁₋₈alkyl,
(3) —C₂₋₈alkenyl,
(4) —C₂₋₈alkynyl,
(5) —C₁₋₈alkoxy,
(6) —(CH₂)ₙC₃₋₇cycloalkyl,
(7) —(CH₂)ₙC₂₋₇heterocycloalkyl,
(8) —(CH₂)ₙ-phenyl,
(9) —(CH₂)ₙ-naphthyl,
(10) —(CH₂)ₙ-heteroaryl, and
(11) —(CH₂)ₙC₃₋₇bicycloalkyl,
wherein alkenyl, alkynyl, phenyl, naphthyl, and heteroaryl are unsubstituted or substituted with one to three groups independently selected from R⁴, and alkyl, alkoxy, cycloalkyl, heterocycloalkyl, and bicycloalkyl are unsubstituted or substituted with one to three groups independently selected from R⁴ and oxo, and wherein any (CH₂) in R⁵ is unsubstituted or substituted with one to two groups independently selected from halogen, hydroxy, and —C₁₋₆alkyl, or two R⁵ groups together with the atom to which they are attached form a 5- to 8-membered mono- or bicyclic ring system optionally containing an additional heteroatom selected from O, S, and —NC₁₋₄ alkyl;
each R⁶ is independently selected from the group consisting of:
(1) hydrogen,
(2) —C₁₋₆alkyl,
(3) —(CH₂)ₙ-phenyl,
(4) —(CH₂)ₙ-heteroaryl,
(5) —(CH₂)ₙ-naphthyl,
(6) —(CH₂)ₙ-heterocycloalkyl,
(7) —(CH₂)ₙC₃₋₇cycloalkyl,
(8) —(CH₂)ₙC₃₋₇bicycloalkyl,
(9) —(CH₂)ₙCF₃, and
(10) —(CH₂)ₙCHF₂,
wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy, or two R⁶ groups together with the atom to which they are attached form a 4- to 8-membered mono- or bicyclic ring system optionally containing an additional heteroatom selected from O, S, and NC₁₋₄ alkyl;
each R⁷ is independently selected from the group consisting of:
(1) hydrogen, and
(2) —C₁₋₈alkyl,
wherein alkyl is unsubstituted or substituted with one to three groups independently selected from halogen, —C₁₋₆alkyl, hydroxy, and C₁₋₄ alkoxy;
each R⁸ is independently selected from the group consisting of:
(1) —C₁₋₆alkyl,
(2) —(CH₂)ₙphenyl,
(3) —(CH₂)ₙnaphthyl,
(4) —(CH₂)ₙheteroaryl,
(5) —(CH₂)ₙC₂₋₇heterocycloalkyl,
(6) —(CH₂)ₙC₃₋₇cycloalkyl,
(7) halogen,
(8) —OR⁶,
(9) —(CH₂)ₙN(R⁶)₂,
(10) —(CH₂)ₙC═N,
(11) —(CH₂)ₙCO₂R⁶,
(12) —NO₂,
(13) —(CH₂)ₙNR⁶S(O)ₚR⁶,
(14) —(CH₂)ₙS(O)ₚN(R⁶)₂,
(15) —(CH₂)ₙS(O)ₚR⁶,
(16) —(CH₂)ₙNR⁶C(O)N(R⁶)₂,
(17) —(CH₂)ₙC(O)N(R⁶)₂,
(18) —(CH₂)ₙNR⁶C(O)R⁶,
(19) —(CH₂)ₙNR⁶CO₂R⁶,
(20) —(CH₂)ₙNR⁶C(O)-heteroaryl,
(21) —(CH₂)ₙC(O)NR⁶N(R⁶)₂,
(22) —(CH₂)ₙC(O)NR⁶NR⁶C(O)R⁶,
(23) —O(CH₂)ₙC(O)N(R⁶)₂,
(24) —CF₃,
(25) —CH₂CF₃,
(26) —OCF₃, and
(27) —OCH₂CF₃,
wherein phenyl, naphthyl, and heteroaryl are unsubstituted or substituted with one to three substituents independently selected from halogen, hydroxy, —C₁₋₆alkyl, trifluoromethyl, and C₁₋₄ alkoxy, and wherein alkyl, cycloalkyl, heterocycloalkyl and (CH₂) are unsubstituted or substituted with one to three substituents independently selected from halogen, hydroxy, oxo, —C₁₋₆alkyl, trifluoromethyl, and C₁₋₄ alkoxy;
each R⁹ is independently selected from the group consisting of:
(1) —(CH₂)ₙ-halogen,
(2) —C₁₋₆alkyl,
(3) —(CH₂)ₙ—CO₂R⁶,
(4) —(CH₂)ₙ—OR⁶,
(5) —(CH₂)ₙ-phenyl,
(6) —(CH₂)ₙ-heteroaryl,
(7) —(CH₂)ₙ-naphthyl,
(8) —(CH₂)ₙ-heterocycloalkyl,
(9) —(CH₂)ₙC₃₋₇cycloalkyl,
(10) —(CH₂)ₙC₃₋₇bicycloalkyl,
(11)-(CH₂)ₙCF₃, and
(12) —(CH₂)ₙCHF₂,
wherein alkyl, phenyl, heteroaryl, heterocycloalkyl, and cycloalkyl are unsubstituted or substituted with one to three groups independently selected from halogen, —C₁₋₆alkyl, hydroxy, and C₁₋₄ alkoxy, or two R⁶ groups together with the atom to which they are attached form a 4- to 8-membered mono- or bicyclic ring system optionally containing an additional heteroatom selected from O, S, and NC₁₋₄ alkyl;
r is 1 or 2;
s is 0, 1, or 2;
n is 0, 1, 2, 3, or 4; and
p is 0, 1, or 2.

13. The compound of claim 1 selected from the group consisting of: 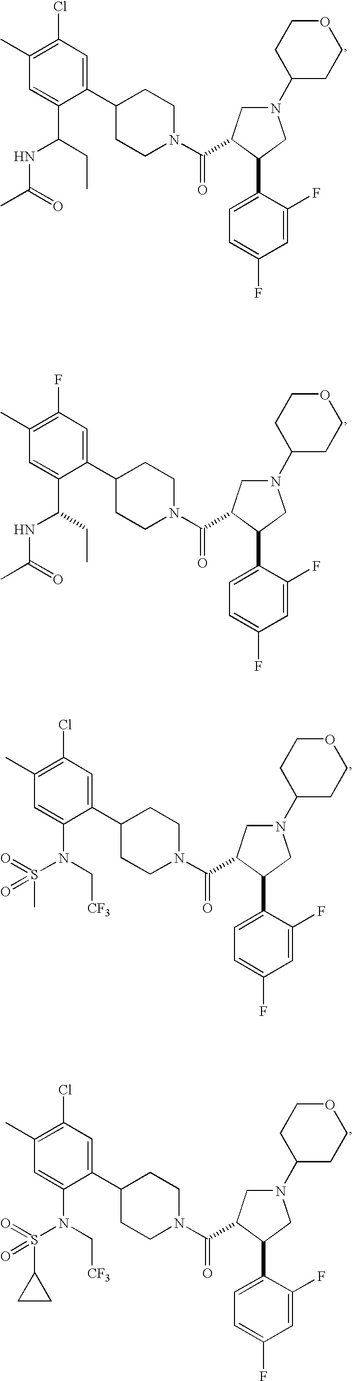 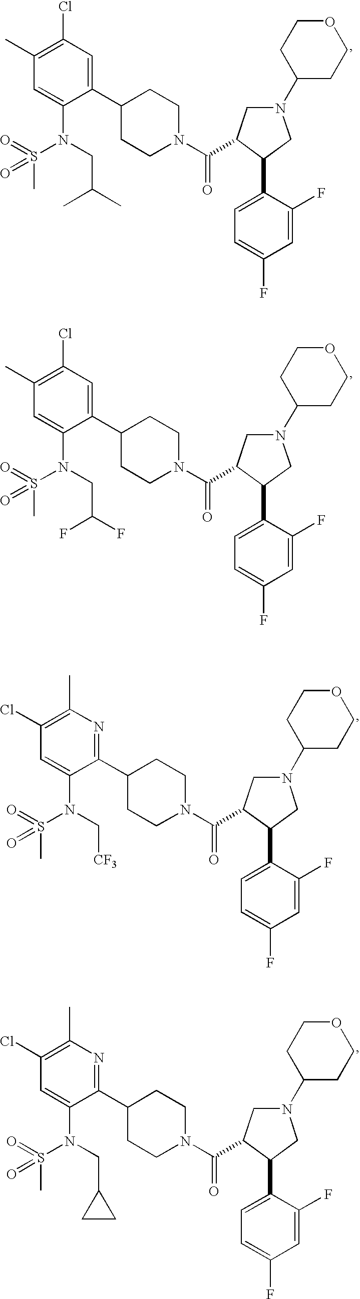 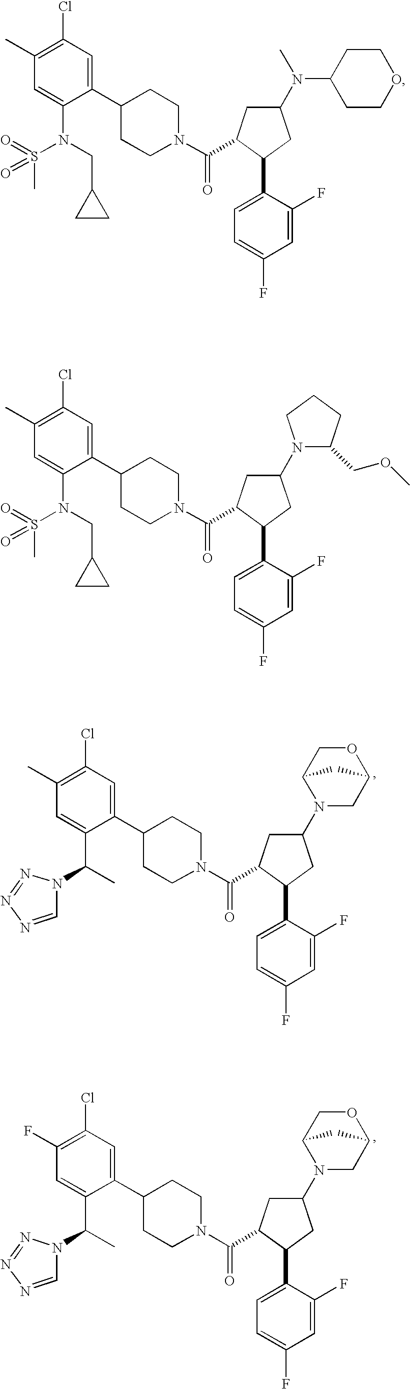 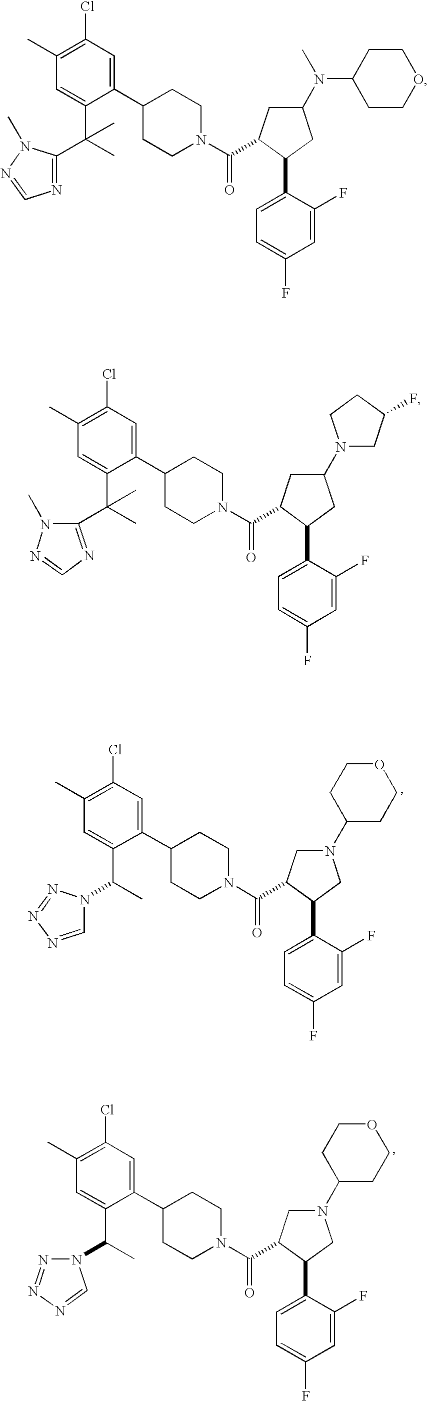 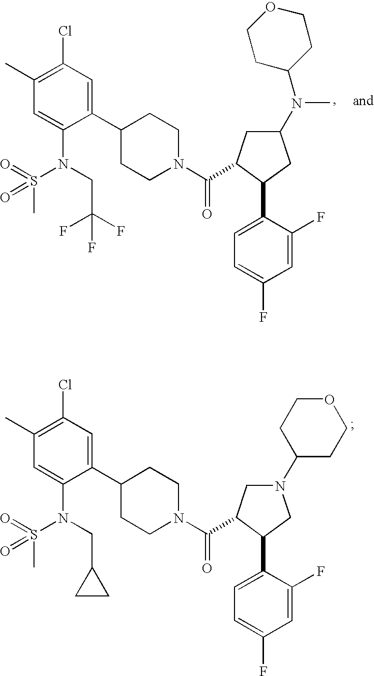
or a pharmaceutically acceptable salt thereof.

14. A composition which comprises a compound of claim 1, or a pharmaceutically acceptable salt thereof, and a pharmaceutically acceptable carrier.

15. (canceled)

16. A method of treating diseases mediated by the melanocortin-4 receptor in a subject in need thereof by administering a therapeutically effective amount of a compound according to claim 1, or a pharmaceutically acceptable salt thereof.

17. The method according to claim 16 wherein the disease mediated by the melanocortin-4 receptor is selected from the group consisting of: obesity, diabetes mellitus, male erectile dysfunction and an obesity-related disorder.
